SEQUENCE LISTING (479 sequences)

SEQ ID NO: 1 - DNA, 2664 bp, Escherichia coli
atgtcagaacgtttcccaaatgacgtggatccgatcgaaactcgcgactggctccaggcgatcgaatcggtcatccgtgaagaaggtgttgagcgtgctcagtatctgatcgaccaactgcttgctgaagcccgcaaaggcggtgtaaacgtagccgcaggcacaggtatcagcaactacatcaacaccatccccgttgaagaacaaccggagtatccgggtaatctggaactggaacgccgtattcgttcagctatccgctggaacgccatcatgacggtgctgcgtgcgtcgaaaaaagacctcgaactgggcggccatatggcgtccttccagtcttccgcaaccatttatgatgtgtgctttaaccacttcttccgtgcacgcaacgagcaggatggcggcgacctggtttacttccagggccacatctccccgggcgtgtacgctcgtgctttcctggaaggtcgtctgactcaggagcagctggataacttccgtcaggaagttcacggcaatggcctctcttcctatccgcacccgaaactgatgccggaattctggcagttcccgaccgtatctatgggtctgggtccgattggtgctatttaccaggctaaattcctgaaatatctggaacaccgtggcctgaaagatacctctaaacaaaccgtttacgcgttcctcggtgacggtgaaatggacgaaccggaatccaaaggtgcgatcaccatcgctacccgtgaaaaactggataacctggtcttcgttatcaactgtaacctgcagcgtcttgacggcccggtcaccggtaacggcaagatcatcaacgaactggaaggcatcttcgaaggtgctggctggaacgtgatcaaagtgatgtggggtagccgttgggatgaactgctgcgtaaggataccagcggtaaactgatccagctgatgaacgaaaccgttgacggcgactaccagaccttcaaatcgaaagatggtgcgtacgttcgtgaacacttcttcggtaaatatcctgaaaccgcagcactggttgcagactggactgacgagcagatctgggcactgaaccgtggtggtcacgatccgaagaaaatctacgctgcattcaagaaagcgcaggaaaccaaaggcaaagcgacagtaatccttgctcataccattaaaggttacggcatgggcgacgcggctgaaggtaaaaacatcgcgcaccaggttaagaaaatgaacatggacggtgtgcgtcatatccgcgaccgtttcaatgtgccggtgtctgatgcagatatcgaaaaactgccgtacatcaccttcccggaaggttctgaagagcatacctatctgcacgctcagcgtcagaaactgcacggttatctgccaagccgtcagccgaacttcaccgagaagcttgagctgccgagcctgcaagacttcggcgcgctgttggaagagcagagcaaagagatctctaccactatcgctttcgttcgtgctctgaacgtgatgctgaagaacaagtcgatcaaagatcgtctggtaccgatcatcgccgacgaagcgcgtactttcggtatggaaggtctgttccgtcagattggtatttacagcccgaacggtcagcagtacaccccgcaggaccgcgagcaggttgcttactataaagaagacgagaaaggtcagattctgcaggaagggatcaacgagctgggcgcaggttgttcctggctggcagcggcgacctcttacagcaccaacaatctgccgatgatcccgttctacatctattactcgatgttcggcttccagcgtattggcgatctgtgctgggcggctggcgaccagcaagcgcgtggcttcctgatcggcggtacttccggtcgtaccaccctgaacggcgaaggtctgcagcacgaagatggtcacagccacattcagtcgctgactatcccgaactgtatctcttacgacccggcttacgcttacgaagttgctgtcatcatgcatgacggtctggagcgtatgtacggtgaaaaacaagagaacgtttactactacatcactacgctgaacgaaaactaccacatgccggcaatgccggaaggtgctgaggaaggtatccgtaaaggtatctacaaactcgaaactattgaaggtagcaaaggtaaagttcagctgctcggctccggttctatcctgcgtcacgtccgtgaagcagctgagatcctggcgaaagattacggcgtaggttctgacgtttatagcgtgacctccttcaccgagctggcgcgtgatggtcaggattgtgaacgctggaacatgctgcacccgctggaaactccgcgcgttccgtatatcgctcaggtgatgaacgacgctccggcagtggcatctaccgactatatgaaactgttcgctgagcaggtccgtacttacgtaccggctgacgactaccgcgtactgggtactgatggcttcggtcgttccgacagccgtgagaacctgcgtcaccacttcgaagttgatgcttcttatgtcgtggttgcggcgctgggcgaactggctaaacgtggcgaaatcgataagaaagtggttgctgacgcaatcgccaaattcaacatcgatgcagataaagttaacccgcgtctggcgtaa

SEQ ID NO: 2 - protein, 887 aa, Escherichia coli
MSERFPNDVDPIETRDWLQAIESVIREEGVERAQYLIDQLLAEARKGGVNVAAGTGISNYINTIPVEEQPEYPGNLELERRIRSAIRWNAIMTVLRASKKDLELGGHMASFQSSATIYDVCFNHFFRARNEQDGGDLVYFQGHISPGVYARAFLEGRLTQEQLDNFRQEVHGNGLSSYPHPKLMPEFWQFPTVSMGLGPIGAIYQAKFLKYLEHRGLKDTSKQTVYAFLGDGEMDEPESKGAITIATREKLDNLVFVINCNLQRLDGPVTGNGKIINELEGIFEGAGWNVIKVMWGSRWDELLRKDTSGKLIQLMNETVDGDYQTFKSKDGAYVREHFFGKYPETAALVADWTDEQIWALNRGGHDPKKIYAAFKKAQETKGKATVILAHTIKGYGMGDAAEGKNIAHQVKKMNMDGVRHIRDRFNVPVSDADIEKLPYITFPEGSEEHTYLHAQRQKLHGYLPSRQPNFTEKLELPSLQDFGALLEEQSKEISTTIAFVRALNVMLKNKSIKDRLVPIIADEARTFGMEGLFRQIGIYSPNGQQYTPQDREQVAYYKEDEKGQILQEGINELGAGCSWLAAATSYSTNNLPMIPFYIYYSMFGFQRIGDLCWAAGDQQARGFLIGGTSGRTTLNGEGLQHEDGHSHIQSLTIPNCISYDPAYAYEVAVIMHDGLERMYGEKQENVYYYITTLNENYHMPAMPEGAEEGIRKGIYKLETIEGSKGKVQLLGSGSILRHVREAAEILAKDYGVGSDVYSVTSFTELARDGQDCERWNMLHPLETPRVPYIAQVMNDAPAVASTDYMKLFAEQVRTYVPADDYRVLGTDGFGRSDSRENLRHHFEVDASYVVVAALGELAKRGEIDKKVVADAIAKFNIDADKVNPRLA

SEQ ID NO: 3 - DNA, 1893 bp, Escherichia coli
atggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaa

SEQ ID NO: 4 - protein, 630 aa, Escherichia coli
MAIEIKVPDIGADEVEITEILVKVGDKVEAEQSLITVEGDKASMEVPSPQAGIVKEIKVSVGDKTQTGALIMIFDSADGAADAAPAQAEEKKEAAPAAAPAAAAAKDVNVPDIGSDEVEVTEILVKVGDKVEAEQSLITVEGDKASMEVPAPFAGTVKEIKVNVGDKVSTGSLIMVFEVAGEAGAAAPAAKQEAAPAAAPAPAAGVKEVNVPDIGGDEVEVTEVMVKVGDKVAAEQSLITVEGDKASMEVPAPFAGVVKELKVNVGDKVKTGSLIMIFEVEGAAPAAAPAKQEAAAPAPAAKAEAPAAAPAAKAEGKSEFAENDAYVHATPLIRRLAREFGVNLAKVKGTGRKGRILREDVQAYVKEAIKRAEAAPAATGGGIPGMLPWPKVDFSKFGEIEEVELGRIQKISGANLSRNWVMIPHVTHFDKTDITELEAFRKQQNEEAAKRKLDVKITPVVFIMKAVAAALEQMPRFNSSLSEDGQRLTLKKYINIGVAVDTPNGLVVPVFKDVNKKGIIELSRELMTISKKARDGKLTAGEMQGGCFTISSIGGLGTTHFAPIVNAPEVAILGVSKSAMEPVWNGKEFVPRLMLPISLSFDHRVIDGADGARFITIINNTLSDIRRLVM

SEQ ID NO: 5 - DNA, 1425 bp, Escherichia coli
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaa

SEQ ID NO: 6 - protein, 474 aa, Escherichia coli [sequence omitted]

SEQ ID NO: 7 - DNA, 1116 bp, Enterococcus faecalis
atggcaaaggctaagaaacaaaaacctattgactttaaagagctaatggctaaagtcgacgctgatttcccaactttccaaatcttggatcaagatggaaaaattgtgaatgaagatttagtacctgatttatcggatgaggaattagttgaattaatgacacgcatggtttggtctcgtgtgttagaccaacgttctactgcattaaaccgtcaaggacgcttaggattcttcgcgccaacagctggacaagaagcaagccaattggcaagtcaatttgcaatggaaaaagaagactacttactaccaggttaccgtgatgtacctcaattagtacaacatggtttaccattaagagaagctttcttatggtctcgtggtcacgtagcagggaactactacgcggaagatttaaatgcattaccaccacaaattatcattggtgctcaatacatccaagcagctggtgttgctttaggattgaaaaaacgtggaaaagaaaatgttgtcttcacttatactggtgacggcggttcttcacaaggggacttctatgaagcaattaactttgctggtgcttaccaagcaaacggtgtcttcattatccaaaacaatggttttgcgatttctacacctcgtgaaaaacaaacagcggctaaaactttagctcaaaaagctgttgcagcaggaattcctggtattcaagttgatggtatggatccattagcagtttacgcaattgcaaaagaagcacgcgattggtcagctgcaggaaacggtccagttttaattgaaacattaacttatcgttatggtccacatactttatctggagacgatccaacacgttaccgttcaaaagaaatggatgacgaatgggtacaaaaagatccattgactcgtttccgtaaatatctaacagataaaggcttatggtctgaagcaaaagaagaagaaattattgaaaaaacaaaagaagaaatcaaagtagcgattgcagaagcggataaagcgccaaaacaaaaagtttctgatttcttgaaaaatatgtttgaagttcaacctcaaacaattaaagaacaaattgcattttatgaagcgaaggagtcgaaataa

SEQ ID NO: 8 - protein, 371 aa, Enterococcus faecalis
MAKAKKQKPIDFKELMAKVDADFPTFQILDQDGKIVNEDLVPDLSDEELVELMTRMVWSRVLDQRSTALNRQGRLGFFAPTAGQEASQLASQFAMEKEDYLLPGYRDVPQLVQHGLPLREAFLWSRGHVAGNYYAEDLNALPPQIIIGAQYIQAAGVALGLKKRGKENVVFTYTGDGGSSQGDFYEAINFAGAYQANGVFIIQNNGFAISTPREKQTAAKTLAQKAVAAGIPGIQVDGMDPLAVYAIAKEARDWSAAGNGPVLIETLTYRYGPHTLSGDDPTRYRSKEMDDEWVQKDPLTRFRKYLTDKGLWSEAKEEEIIEKTKEEIKVAIAEADKAPKQKVSDFLKNMFEVQPQTIKEQIAFYEAKESK

SEQ ID NO: 9 - DNA, 978 bp, Enterococcus faecalis
atggcacaaaaaactatgatccaagcaattacagatgccttagctcttgaattagagaaagacgaaaatgtcttaatcttcggtgaagacgttggtaacaacggtggggttttccgtgcaactgaaggtttacaagaaaaatttggtgaagaccgcgtcttcgatacacctttagctgaatctggtatcggtggattggctttcggtcttgccttgcaaggttaccgtccagttcctgaaatccaattctttggtttcgtttttgaagtatttgacgaaatcgttggtcaaatggctcgtacgcgttaccgtatgggtggaactcgtaatatgccaattactgttcgtgccccatttggtggtggtgttcatacaccagaacttcactcagataacttagaaggattaatcgcacaatcaccaggtgttcgtgttgttattccatcaaacccttacgatgcaaaaggactattaatttcatctattcgtagcaacgatccagttgtttacttagagcacatgaaattataccgttcattccgtgaggaagtgccagacgaagcttatgaagtgcctttagataaagcggctgtaactcgtgaaggaacagacgtatcaatcatcacttacggtgctatggttcgtgaagcgattaaagcagctgatagcttagcgaaagacaatatttcagcagaaatcattgacttacgtacagtggctcctttagatgtggaaacaattattaactctgttgaaaaaactggccgtgtggttgtcgttcaagaagcacaaaaacaagctggcgttggcgctatggttgtttctgaaatttctgaacgtgccgtattatcattagaagcaccaatcggacgtgtatctgctccagatacaatcttcccattcggacaagcagaaaatatctggttaccaaatgcgaaagatatcgaagcaaaagctagagaaatcgtcgaattttaa

SEQ ID NO: 10 - protein, 325 aa, Enterococcus faecalis
MAQKTMIQAITDALALELEKDENVLIFGEDVGNNGGVFRATEGLQEKFGEDRVFDTPLAESGIGGLAFGLALQGYRPVPEIQFFGFVFEVFDEIVGQMARTRYRMGGTRNMPITVRAPFGGGVHTPELHSDNLEGLIAQSPGVRVVIPSNPYDAKGLLISSIRSNDPVVYLEHMKLYRSFREEVPDEAYEVPLDKAAVTREGTDVSIITYGAMVREAIKAADSLAKDNISAEIIDLRTVAPLDVETIINSVEKTGRVVVVQEAQKQAGVGAMVVSEISERAVLSLEAPIGRVSAPDTIFPFGQAENIWLPNAKDIEAKAREIVEF

SEQ ID NO: 11 - DNA, 1620 bp, Enterococcus faecalis
atggcttatcagtttaaattaccggatatcggtgaagggattgccgaaggcgaaatcgttaaatggtttgtaaaacctggcgatacaatcaacgaagacgatacgttattagaagtacaaaatgacaaatcagtggaagaaattccatcaccagtaacaggtactgtaaaaaatatcgttgtaccagaaggaacagttgcaaacgttggtgacgtgttaatcgaaatcgacgcacctggtcacgaagataacgatgcagcaccagcagctcctgcacaagaacaaacaccagcacaacctgctgctgtaccaacaaccgaagcagctggcggatttttccaattcaaattaccagacatcggtgaaggaattgccgaaggcgaaatcgttaaatggttcgttaaagcgggcgacacaattaatgaagatgattcattattagaagtacaaaatgacaaatcagtagaagaaattccatcaccagtaacaggtactgtaaaaaatatcgttgtaccagaaggaacagttgccaatgtgggtgacgtgttagttgaaattgacgcacctggtcataattcagcagcaccgtcagtcgcagcaccagctactgacgctcctaaagcggaagcatcagctccagccgcttcaacaggcgtagttgcagccgctgatccaaacaaacgcgttttagcaatgccatctgttcgtcagtatgcgcgtgaaaaagacgttgatattacacaagtaactgcaactggtaaaggtggccgtgtcattaaagcggatattgatgcctttgtctctggtggttctcaagcagccccagctactgaagctgccgcaacagaagcagcacctaaagcggaagcagctgcacctaaagcagcgccaaaagcctttacttctgatttaggcgaaatggaaacacgtgaaaaaatgacaccaacacgtaaagcaattgctaaagcaatggttaacagcaaacacactgctcctcacgtaacattacatgatgaagtagaagtttctaaattatgggatcaccgtaagaaatttaaagatgttgctgctgcaaatggtacaaaattaacattcttaccatacgttgtaaaagcattgacttcaactgttcaaaaattcccaatcttgaatgcatcaatcgatgacgcagcacaagaaattgtttacaaaaattactttaacattggtatcgctactgatacagatcatggcttatatgtaccaaatgttaaaaatgctaatacgaagagcatgtttgctatcgctgatgaaatcaacgaaaaagcagcattggctatcgaaggtaaattaactgcacaagatatgcgtgatggtacaatcacaattagtaacattggttcagtcggtggcggctggtttacaccagtaatcaactaccctgaagttgctattttaggcgttggtacaattgcacaagaaccagttgttaatgcagacggcgaaatcgttgtgggacgcatgatgaaattatcattaagctttgaccaccgtatcgttgacggcgcaactgctcaaaaagcaatgaacaacattaaacgcttattagctgatccagaattactattaatggaaggatga

SEQ ID NO: 12 - protein, 539 aa, Enterococcus faecalis
MAYQFKLPDIGEGIAEGEIVKWFVKPGDTINEDDTLLEVQNDKSVEEIPSPVTGTVKNIVVPEGTVANVGDVLIEIDAPGHEDNDAAPAAPAQEQTPAQPAAVPTTEAAGGFFQFKLPDIGEGIAEGEIVKWFVKAGDTINEDDSLLEVQNDKSVEEIPSPVTGTVKNIVVPEGTVANVGDVLVEIDAPGHNSAAPSVAAPATDAPKAEASAPAASTGVVAAADPNKRVLAMPSVRQYAREKDVDITQVTATGKGGRVIKADIDAFVSGGSQAAPATEAAATEAAPKAEAAAPKAAPKAFTSDLGEMETREKMTPTRKAIAKAMVNSKHTAPHVTLHDEVEVSKLWDHRKKFKDVAAANGTKLTFLPYVVKALTSTVQKFPILNASIDDAAQEIVYKNYFNIGIATDTDHGLYVPNVKNANTKSMFAIADEINEKAALAIEGKLTAQDMRDGTITISNIGSVGGGWFTPVINYPEVAILGVGTIAQEPVVNADGEIVVGRMMKLSLSFDHRIVDGATAQKAMNNIKRLLADPELLLMEG

SEQ ID NO: 13 - DNA, 1407 bp, Enterococcus faecalis
atggtagtaggagatttcgccattgaactagatacagtcgtaatcggagctggtcctggaggatacgttgccgcaattcgtgccgcagaaatgggtcaaaaagttgcgattatcgaacgtgaatacatcggaggcgtttgtttaaacgttggatgtattccttcaaaagctttaattgctgctggacatcattaccaagaagcacaagattcttcaacttttggtgtaacagctaaaggagtcgaattagactttgcaaaaacacaagactggaaagataacacagttgtaaaatcattaacaggcggcgttggcatgttattgaaaaaacacaaagtagaaattattgaaggcgaagcattcttcgttgacgaaaatacattgcgtgttattcacccagactcagcacaaacttactcattcaataatgctattgtagcaacaggttctcgtccaattgaaatcccaggattcaaatttggcggacgcgtgttagattctacaggcggtttaaacttaaaagaagttcctaaaaaattcgttattatcggtggcggtgtcatcggtgctgaattaggtggcgcttatgctaacttaggttcagaagtaacaattttagaaggtagcccatcaattttaccaacttatgaaaaagatatggttaaagttgtcacagaccacttcaagaagaaaaacgtaacaatcgtgacttctgcaatggctaaagaagctgttgacaatggcgatagcgtcactgttaaatatgaagttaacggaaaagaagaaagtgttgaagcagattacgtaatggtcactgttggacgtcgtccaaacacagacgacttaggcttagaacaagcgggcgttgaaattggcgaacgtggtttaatcccagttgacaaccaaggacgtactaacgtgaaaaacatcttcgcaatcggcgacatcgtaccaggtgctgcgttagcgcataaagcaagctacgaagcaaaaattgctgctgaagcaatttctggtaagaaagttgcagttgattacaaagcaatgccagctgttgcctttactgatccagaattggcaagcgttggtatgactgttgcagaagcaaaagaagcgggaatcgaagcaaaaggctacaaattcccatttgctggtaacggccgtgcaatctctttagataaaactgaaggattcatgcgtttagttacaactgtagaagacaatgtcatcatcggtgcacaaattgccggtgtcggtgcaagtgacatgatttctgaattagctttagctattgaatctggcatgaatgcagaagacattgctttaacaatccacccacacccatcattgggcgaaattactatggatacagctgaattggctttaggtttaccaattcatatttaa

SEQ ID NO: 14 - protein, 468 aa, Enterococcus faecalis
MVVGDFAIELDTVVIGAGPGGYVAAIRAAEMGQKVAIIEREYIGGVCLNVGCIPSKALIAAGHHYQEAQDSSTFGVTAKGVELDFAKTQDWKDNTVVKSLTGGVGMLLKKHKVEIIEGEAFFVDENTLRVIHPDSAQTYSFNNAIVATGSRPIEIPGFKFGGRVLDSTGGLNLKEVPKKFVIIGGGVIGAELGGAYANLGSEVTILEGSPSILPTYEKDMVKVVTDHFKKKNVTIVTSAMAKEAVDNGDSVTVKYEVNGKEESVEADYVMVTVGRRPNTDDLGLEQAGVEIGERGLIPVDNQGRTNVKNIFAIGDIVPGAALAHKASYEAKIAAEAISGKKVAVDYKAMPAVAFTDPELASVGMTVAEAKEAGIEAKGYKFPFAGNGRAISLDKTEGFMRLVTTVEDNVIIGAQIAGVGASDMISELALAIESGMNAEDIALTIHPHPSLGEITMDTAELALGLPIHI

SEQ ID NO: 15 - DNA, 1182 bp, Rastonia eutrophus
atgactgatgttgttatcgtttctgcggcccgtactgctgttggtaagttcggtggctccctggcaaaaattccggctccggaactgggcgcagtagttatcaaggcggcactggagcgtgccggtgtgaaaccggaacaagtgagcgaggtaattatgggtcaagtgctgactgcgggtagcggtcaaaacccggctcgtcaggcggccatcaaagcaggtctgccggctatggtgccggcgatgacgatcaacaaagtttgtggctctggcctgaaagctgtgatgctggcggctaacgcgattatggcaggcgacgccgaaattgtcgttgctggtggtcaggagaatatgtctgcggcaccgcatgttctgccgggttcccgtgatggcttccgcatgggtgatgcaaaactggttgacactatgatcgtcgatggtctgtgggacgtatacaaccagtaccacatgggtatcactgccgaaaacgttgctaaagagtatggcatcacccgcgaagcccaagatgaatttgcagtcggctctcaaaacaaagcggaagcggcgcagaaagctggcaaattcgatgaggagattgttccggttctgattccacagcgcaaaggcgacccggtggcctttaaaaccgacgagtttgtgcgtcaaggcgcaaccctggattctatgtccggtctgaaaccggccttcgacaaagctggtactgttactgcggcgaacgcaagcggtctgaacgatggtgcggcggcagttgtggttatgagcgcggcaaaagcaaaagaactgggtctgaccccgctggccaccatcaaatcttacgctaacgcgggcgttgacccgaaagtgatgggcatgggcccggttccggcgtctaaacgcgcactgagccgtgcggagtggactccgcaagacctggatctgatggaaattaacgaagcatttgcggctcaagcactggccgttcatcagcagatgggctgggacaccagcaaagttaacgttaatggtggtgcaatcgctattggccacccaatcggtgcttctggttgccgtatcctggtgacgctgctgcatgaaatgaaacgccgtgatgctaaaaagggcctggcaagcctgtgcattggtggtggtatgggtgtagcactggctgtcgaacgcaaataa

SEQ ID NO: 16 - protein, 393 aa, Rastonia eutrophus
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 17 - DNA, 741 bp, Rastonia eutrophus
atgacccagcgcatcgcttacgtaaccggtggcatgggtggtattggcaccgcaatctgtcagcgtctggccaaagacggctttcgtgttgttgcgggttgcggtccgaactctccgcgccgtgaaaaatggctggaacagcaaaaagcgctgggttttgacttcatcgcaagcgaaggtaatgttgcggactgggattctaccaagaccgcattcgacaaagttaaatctgaagtaggcgaggtagatgtcctgatcaacaacgcaggtattacccgtgatgttgtgttccgtaaaatgacgcgtgctgactgggacgcggtgatcgataccaacctgacttccctgtttaacgtgacgaaacaagttattgacggtatggctgaccgcggttggggccgtatcgtgaatatctcttccgtgaacggccaaaaaggtcaattcggccagactaactactctaccgcgaaagcaggtctgcacggttttactatggcgctggctcaggaagttgcgactaaaggtgttacggttaacaccgtatctccgggttacattgctactgatatggttaaagctattcgtcaggatgttctggacaagatcgtcgccacgatcccggtgaagcgcctgggcctgccggaggaaatcgcaagcatctgtgcatggctgtcttccgaagaatccggtttctccacgggtgcagacttttccctgaacggtggcctgcacatgggctaa

SEQ ID NO: 18 - protein, 246 aa, Rastonia eutrophus
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 19 - DNA, 849 bp, Clostridium acetobutylicum
atgaaaaaggtttgcgttattggtgcgggtactatgggttctggtatcgcccaggctttcgcagcaaagggcttcgaagtcgttctgcgcgacattaaggatgaatttgttgatcgcggcctggacttcattaacaaaaacctgtctaaactggtaaagaaaggtaaaatcgaggaggctacgaaagtagaaatcctgacccgtatttccggcaccgttgacctgaacatggctgccgattgtgacctggttatcgaagcagcagtagaacgcatggacatcaagaaacagattttcgctgatctggacaacatctgcaagcctgaaacgatcctggcgtctaacacttcttccctgagcatcactgaagtcgcttccgcaaccaaacgtccggataaggtgattggtatgcacttctttaacccggcaccagttatgaaactggtcgaagtgatccgtggcatcgctacttctcaggaaaccttcgacgcggtgaaggaaacttccatcgccatcggtaaagatccggtagaagttgctgaagcaccgggtttcgtagttaaccgtattctgatcccgatgattaacgaagcagtaggcatcctggctgagggtatcgcatctgttgaagacatcgacaaggcaatgaagctgggtgcaaatcaccctatgggcccgctggagctgggtgattttatcggcctggacatctgtctggccatcatggacgtactgtactctgaaacgggcgattctaagtaccgtccgcacacgctgctgaaaaagtatgttcgtgccggctggctgggtcgtaaatctggcaaaggtttttacgattactccaagtaa

SEQ ID NO: 20 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 21 - DNA, 786 bp, Clostridium acetobutylicum
atggaactgaacaacgtgatcctggaaaaagagggtaaagtagcagttgtcaccatcaaccgtccaaaagcgctgaatgctctgaactccgacaccctgaaagaaatggactacgttattggcgaaatcgagaacgacagcgaagtgctggcagttatcctgactggcgctggcgaaaagagcttcgttgcgggcgcagacatctctgaaatgaaagaaatgaataccatcgaaggccgtaaattcggtattctgggtaacaaagtcttccgccgtctggaactgctggaaaagccggttattgcggcggtgaacggcttcgccctgggtggcggttgtgaaatcgcgatgagctgcgacatccgtatcgcatcttctaacgctcgctttggtcaaccggaagttggcctgggcattacgccgggttttggtggcactcagcgtctgtctcgtctggtaggtatgggtatggctaaacagctgattttcaccgctcagaacatcaaagctgacgaggcactgcgcatcggtctggtgaacaaggttgtggagccgtccgagctgatgaataccgcgaaagaaatcgcgaacaaaatcgtgtctaacgcgccggtggccgtaaaactgtccaagcaggcaatcaatcgtggtatgcagtgcgacattgacactgcgctggctttcgaatctgaagcattcggcgaatgcttctccactgaggatcagaaggatgccatgaccgctttcatcgaaaaacgtaaaatcgaaggcttcaaaaatcgttaa

SEQ ID NO: 22 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 23 - DNA, 1344 bp, Streptomyces cinnamonensis
atgacggtaaaagatattctggacgcgattcagtccaaagacgctacctctgccgactttgcggcactgcaactgccggaatcttaccgtgcaattacggttcacaaagacgaaacggagatgttcgcgggcctggaaacccgcgataaagacccgcgcaaatctattcacctggacgaggtgccggttccggaactgggccctggcgaggcactggttgcggttatggcctctagcgtgaactacaactctgtttggacgtctatttttgagccggttagcacgttcgcattcctggaacgttatggtaaactgagcccgctgaccaaacgtcacgacctgccataccatatcatcggttccgacctggcgggtgttgtactgcgcactggcccaggtgttaacgcatggcaaccgggtgacgaggttgtggcgcattgcctgagcgtggaactggaatccccggacggccacgatgacacgatgctggaccctgaacagcgcatttggggtttcgaaactaacttcggtggtctggctgaaatcgcgctggtcaagaccaaccaactgatgccgaaaccgaaacacctgacttgggaagaagcggcggctcctggcctggttaattctactgcgtatcgccaactggtttcccgcaatggtgcggcaatgaaacagggcgataacgtactgatttggggcgcttccggcggtctgggttcttatgcaacccagttcgctctggccggtggcgctaacccgatctgcgtggtgtcctctccacagaaagcagaaatctgccgtagcatgggtgccgaggctatcatcgaccgtaatgccgaaggctacaaattttggaaagatgaacatacccaagaccctaaggaatggaaacgttttggtaaacgtatccgtgagctgactggcggcgaagatattgacattgtttttgaacacccaggccgtgaaacctttggcgcgagcgtttacgttacccgtaaaggtggtactattaccacgtgcgcttccacctctggttacatgcacgaatatgacaaccgctacctgtggatgagcctgaaacgtatcattggtagccacttcgcaaactaccgtgaagcgtacgaagctaaccgtctgatcgctaaaggcaaaatccacccaacgctgtctaaaacctattccctggaagaaaccggccaggcggcctatgacgttcaccgtaacctgcaccagggcaaagtaggtgttctgtgtctggccccggaggagggtctgggcgtacgtgacgccgaaatgcgcgcccagcatatcgacgcgatcaaccgttttcgtaatgtgtaa

SEQ ID NO: 24 - protein, 453 aa, Streptomyces cinnamonensis
MKEILDAIQAQTATASGTAAVTSADFAALPLPDSYRAITVHKDETEMFAGLESRDKDPRKSLHLDDVPIPELGPGEALVAVMASSVNYNSVWTSIFEPVSTFSFLERYGRLSDLSKRHDLPYHIIGSDLAGVVLRTGPGVNAWNPGDEVVAHCLSVELESSDGHNDTMLDPEQRIWGFETNFGGLAEIALVKSNQLMPKPGHLSWEEAASPGLVNSTAYRQLVSRNGAGMKQGDNVLIWGASGGLGSYATQFALAGGANPICVVSSPQKAEICRAMGAEAIIDRNAEGYKFWKDEQTQDPKEWKRFGKRIRELTGRRGLDIVFEHPGRETFGASVYVTRKGGTITTCASTSGYMHEYDNRYLWMSLKRIIGSHFANYREAWEANRLIAKGKIHPTLSKTYRLEDTGQAAYDVHRNLHQGKVGVLALAPEEGLGVRDPEKRAQHIDAINRFRNV

SEQ ID NO: 25 - DNA, 1344 bp, Streptomyces collinus; identical to SEQ ID NO: 23

SEQ ID NO: 26 - protein, 447 aa, Streptomyces collinus
MTVKDILDAIQSKDATSADFAALQLPESYRAITVHKDETEMFAGLETRDKDPRKSIHLDEVPVPELGPGEALVAVMASSVNYNSVWTSIFEPVSTFAFLERYGKLSPLTKRHDLPYHIIGSDLAGVVLRTGPGVNAWQPGDEVVAHCLSVELESPDGHDDTMLDPEQRIWGFETNFGGLAEIALVKTNQLMPKPKHLTWEEAAAPGLVNSTAYRQLVSRNGAAMKQGDNVLIWGASGGLGSYATQFALAGGANPICVVSSPQKAEICRSMGAEAIIDRNAEGYKFWKDEHTQDPKEWKRFGKRIRELTGGEDIDIVFEHPGRETFGASVYVTRKGGTITTCASTSGYMHEYDNRYLWMSLKRIIGSHFANYREAYEANRLIAKGKIHPTLSKTYSLEETGQAAYDVHRNLHQGKVGVLCLAPEEGLGVRDAEMRAQHIDAINRFRNV

SEQ ID NO: 27 - DNA, 1140 bp, Clostridium acetobutylicum
atggattttaatttaacaagagaacaagaattagtaagacagatggttagagaatttgctgaaaatgaagttaaacctatagcagcagaaattgatgaaacagaaagatttccaatggaaaatgtaaagaaaatgggtcagtatggtatgatgggaattccattttcaaaagagtatggtggcgcaggtggagatgtattatcttatataatcgccgttgaggaattatcaaaggtttgcggtactacaggagttattctttcagcacatacatcactttgtgcttcattaataaatgaacatggtacagaagaacaaaaacaaaaatatttagtacctttagctaaaggtgaaaaaataggtgcttatggattgactgagccaaatgcaggaacagattctggagcacaacaaacagtagctgtacttgaaggagatcattatgtaattaatggttcaaaaatattcataactaatggaggagttgcagatacttttgttatatttgcaatgactgacagaactaaaggaacaaaaggtatatcagcatttataatagaaaaaggcttcaaaggtttctctattggtaaagttgaacaaaagcttggaataagagcttcatcaacaactgaacttgtatttgaagatatgatagtaccagtagaaaacatgattggtaaagaaggaaaaggcttccctatagcaatgaaaactcttgatggaggaagaattggtatagcagctcaagctttaggtatagctgaaggtgctttcaacgaagcaagagcttacatgaaggagagaaaacaatttggaagaagccttgacaaattccaaggtcttgcatggatgatggcagatatggatgtagctatagaatcagctagatatttagtatataaagcagcatatcttaaacaagcaggacttccatacacagttgatgctgcaagagctaagcttcatgctgcaaatgtagcaatggatgtaacaactaaggcagtacaattatttggtggatacggatatacaaaagattatccagttgaaagaatgatgagagatgctaagataactgaaatatatgaaggaacttcagaagttcagaaattagttatttcaggaaaaatttttagataa

SEQ ID NO: 28 - protein, 379 aa, Clostridium acetobutylicum
MDFNLTREQELVRQMVREFAENEVKPIAAEIDETERFPMENVKKMGQYGMMGIPFSKEYGGAGGDVLSYIIAVEELSKVCGTTGVILSAHTSLCASLINEHGTEEQKQKYLVPLAKGEKIGAYGLTEPNAGTDSGAQQTVAVLEGDHYVINGSKIFITNGGVADTFVIFAMTDRTKGTKGISAFIIEKGFKGFSIGKVEQKLGIRASSTTELVFEDMIVPVENMIGKEGKGFPIAMKTLDGGRIGIAAQALGIAEGAFNEARAYMKERKQFGRSLDKFQGLAWMMADMDVAIESARYLVYKAAYLKQAGLPYTVDAARAKLHAANVAMDVTTKAVQLFGGYGYTKDYPVERMMRDAKITEIYEGTSEVQKLVISGKIFR

SEQ ID NO: 29 - DNA, 1194 bp, Treponema denticola
atgatcgtcaagccaatggtgcgcaataatatctgtctgaacgctcacccgcagggttgtaaaaagggtgtagaagaccagattgaatacactaagaaacgcatcaccgcagaagttaaagcaggtgccaaagcaccgaaaaacgtcctggtgctgggctgcagcaacggctacggtctggcaagccgcattacggctgcattcggttacggcgctgctactattggtgttagcttcgaaaaggcgggttctgaaaccaaatacggcactccaggctggtacaacaacctggcattcgacgaagcagcgaagcgtgagggtctgtactctgttaccatcgacggtgacgcgttctctgacgagatcaaagctcaggttatcgaggaagctaaaaagaaaggtatcaaattcgacctgattgtgtactccctggcctctccggttcgtaccgacccggataccggcatcatgcacaaaagcgtactgaagccgtttggcaaaaccttcactggtaaaaccgttgatcctttcaccggcgagctgaaggaaatctccgccgagccagctaacgatgaggaggctgctgcgaccgttaaagtgatgggtggcgaagactgggaacgttggatcaaacaactgtccaaggaaggtctgctggaggagggctgtattactctggcatattcttacatcggcccggaggcgactcaggcactgtatcgtaagggcaccatcggtaaagcgaaagaacatctggaggccaccgctcaccgtctgaacaaggaaaacccgagcatccgtgctttcgtgtccgttaacaagggcctggttacgcgcgcttccgcagtaattccggtcattccgctgtacctggcttccctgtttaaagtcatgaaagaaaaaggcaaccacgaaggttgtatcgaacaaattactcgcctgtatgcggagcgcctgtaccgtaaggatggcactatcccggttgatgaagagaaccgcatccgcattgacgattgggaactggaagaggatgtacagaaagcggtttccgcgctgatggaaaaagtgacgggcgaaaacgcggaatccctgacggatctggcaggttaccgtcacgactttctggcgtctaatggtttcgacgttgagggtattaactacgaggcagaagttgaacgtttcgatcgtatttaa

SEQ ID NO: 30 - protein, 397 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 31 - DNA, 1218 bp, E. gracilis
atggctatgtttaccactaccgcgaaagttatccagccgaaaatccgtggttttatctgcactaccactcacccaattggctgcgaaaaacgcgtccaggaagaaattgcttacgctcgtgcgcacccgccaaccagccctggccctaagcgtgtactggtcatcggttgtagcacgggttacggtctgtctacccgtatcactgctgcgttcggctaccaggcggcgaccctgggcgttttcctggcgggtccaccgaccaaaggtcgcccggcagctgcgggttggtacaacactgttgccttcgagaaagcagcgctggaggcgggcctgtatgcccgttctctgaacggcgacgcttttgattccactacgaaagcgcgcactgttgaagctatcaaacgtgacctgggcaccgtagacctggtagtgtactctatcgctgccccgaagcgtaccgatccggcgaccggcgttctgcacaaggcttgtctgaaaccaatcggcgcgacttacaccaaccgtaccgtcaacaccgacaaagcggaggtgaccgatgttagcatcgaacctgcctccccggaagagatcgcggacacggttaaagtgatgggtggtgaagactgggagctgtggattcaggcgctgagcgaagccggtgttctggcggagggtgcgaaaaccgtggcgtactcctacattggccctgagatgacctggccggtatattggtctggtactattggcgaagccaaaaaggatgttgaaaaggcggctaaacgtatcacccagcagtatggttgcccagcatacccggtagtcgctaaagcgctggtcacccaggccagctccgcaattccggtagttccactgtacatttgcctgctgtaccgtgtgatgaaagaaaaaggtactcatgaaggttgcattgaacagatggttcgtctgctgaccactaaactgtaccctgagaacggtgctccgatcgtggacgaagcgggccgtgttcgtgttgatgactgggaaatggctgaagacgtgcagcaagctgttaaagacctgtggtcccaggtgtctacggctaacctgaaagacatcagcgacttcgctggctaccaaactgagttcctgcgtctgtttggttttggtatcgacggtgtagactacgaccagccggttgacgttgaagcggacctgccgagcgcagcgcagcaataa

SEQ ID NO: 32 - protein, 405 aa, E. gracilis
MAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 33 - DNA, 2577 bp, Clostridium acetobutylicum
atgaaagtcacgaaccagaaggaactgaagcagaaactgaacgaactgcgcgaagcacaaaagaaattcgctacctacacccaggaacaggtggacaaaattttcaagcaatgcgcaatcgcggctgcaaaagaacgtatcaacctggcaaaactggcggtggaagagactggtattggtctggttgaagataaaatcatcaaaaaccacttcgcggctgagtacatctacaacaaatacaaaaacgaaaagacttgtggtatcatcgatcacgatgactccctgggtattaccaaagtagctgaaccgatcggcatcgttgctgcgatcgtaccgaccaccaacccgacttccactgctatcttcaaatccctgatttccctgaaaacgcgcaacgcaatctttttcagccctcacccgcgtgctaaaaagagcactatcgcagccgccaaactgattctggacgccgcagtcaaagcaggtgcgccgaaaaatatcatcggctggatcgatgaaccttctatcgaactgtcccaggatctgatgtccgaagctgatatcattctggctaccggtggtccgagcatggttaaggcggcttacagcagcggtaaacctgccatcggcgtgggtgccggtaacaccccggcgatcatcgatgagtctgctgacatcgatatggcagtatcttccattattctgtccaagacttacgataacggtgttatctgcgcaagcgaacagtccatcctggttatgaactccatctacgaaaaagtaaaggaggaatttgtcaagcgtggtagctatatcctgaaccagaacgaaatcgcgaagatcaaagagacgatgttcaagaacggcgcgatcaacgccgacatcgtgggcaaatccgcctacatcattgcgaagatggcaggtatcgaagttccgcagacgactaaaatcctgatcggtgaagtacagtctgttgaaaagtccgaactgttcagccatgagaaactgagcccggtcctggccatgtataaagttaaagacttcgatgaagctctgaaaaaggcgcaacgtctgatcgagctgggtggttctggtcacacctctagcctgtacatcgactctcaaaataacaaggacaaggtaaaagaatttggtctggctatgaaaacctcccgcaccttcatcaacatgccaagctcccagggtgccagcggtgacctgtacaactttgcaattgcgccgtccttcaccctgggttgcggcacctggggtggcaacagcgtttcccaaaacgtggagccgaagcatctgctgaacatcaaatctgttgcagaacgccgtgaaaacatgctgtggttcaaagtcccacagaaaatttacttcaaatacggctgcctgcgtttcgcgctgaaagaactgaaagacatgaacaaaaagcgtgcgttcattgttaccgacaaagacctgttcaaactgggttacgtgaacaaaatcaccaaagttctggatgaaattgacatcaagtactccatcttcactgatatcaaatccgacccaacgattgatagcgtgaaaaagggcgctaaagaaatgctgaactttgaaccggacaccatcatcagcatcggtggtggctctcctatggatgctgcgaaggtcatgcacctgctgtacgaatacccggaagcggaaatcgaaaacctggctatcaacttcatggacatccgcaaacgtatctgcaacttcccgaagctgggcactaaagctatttccgttgccatcccgactaccgcgggcactggttccgaagccacgccgttcgccgtgatcaccaacgatgaaaccggtatgaaatacccgctgacctcttacgaactgaccccgaacatggcaattatcgacaccgagctgatgctgaacatgccgcgcaagctgaccgctgctaccggcatcgacgctctggtacatgctattgaggcgtacgtttccgtgatggctaccgattacaccgacgaactggccctgcgtgcgatcaaaatgattttcaagtacctgcctcgcgcttacaaaaacggcacgaatgacatcgaggcgcgtgagaaaatggcccatgcaagcaacatcgcgggcatggccttcgccaacgcgttcctgggcgtgtgccactctatggctcacaaactgggtgctatgcaccacgtgccgcacggtatcgcgtgtgctgtcctgatcgaagaagtaattaagtacaacgctactgattgcccgactaaacagaccgccttcccacagtacaaatctcctaacgctaaacgtaagtacgctgagatcgccgaatacctgaacctgaagggtacgagcgacactgagaaagttactgcgctgatcgaagctatctctaaactgaaaattgacctgtccatcccgcagaacatcagcgccgcaggcatcaacaaaaaggacttttacaacacgctggacaaaatgagcgaactggcttttgacgaccagtgcaccactgcaaacccgcgttacccgctgatctctgagctgaaagacatttatatcaaatctttttaa

SEQ ID NO: 34 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 35 - DNA, 3525 bp, Escherichia coli
atgattactattgacggtaatggcgcggttgcttcggtggcgtttcgcaccagtgaagttatcgccatctaccctattacccccagttccacgatggcagaacaggctgatgcctgggccggaaacggcttgaagaacgtttggggagacacaccacgcgtggttgaaatgcagtcggaagcgggtgctatcgctaccgtgcatggcgctttgcagacgggtgccctttcaacatcgtttacgtcatcgcagggtttactgctgatgatcccgacgctgtacaaactggcaggcgaactcacaccgtttgtcctgcatgtagcggcacgtaccgttgccacacatgcactctctatttttggcgatcattccgacgtcatggcggtgcgccagacgggttgcgcgatgttgtgtgcggcaaacgtccaggaagcgcaagactttgctctcatttcgcacatcgcgacgctgaaaagccgcgtgccatttattcatttctttgatggtttccgcacgtcccacgaaatcaataaaattgtcccgctggccgatgacactattcttgatctcatgccgcaggccgaaattgatgctcatcgcgcccgggcgctcaacccggaacatccggtgatccgcggtacgtccgccaatcctgacacttatttccagtctcgcgaagccaccaacccctggtacaacgcggtctatgaccatgttgaacaggcgatgaatgatttcgctgccgcgacaggtcgtcagtatcagccgtttgagtattacgggcatccgcaagcggaacgggtgattatcctgatgggctctgccattggcacctgtgaagaagtgattgatgaattgctaacccgtggcgaaaatgtcggcgtgctgaaagttcgcctataccgccccttctccgctaaacatttactgcaagctctgccgggatccgtacgcagcgtggcggtactggacagaaccaaagaacccggtgcccaggcagaaccgctctatctggatgtgatgaccgcactggcagaagcctttaataatggcgagcgcgaaactctgccccgtgtcattggtgggcgctatggtctttcatccaaagaatttggcccggactgtgtactggcggtatttgccgagctcaacgcggctaaaccgaaagcgcgctttacggttggtatttacgatgatgtgactaatctgtcactgccgttgccggaaaacaccctgccaaactcggcgaaactggaagccttgttttatggccttggtagtgatggcagcgtttccgcgaccaaaaacaatatcaagattatcggtaattccacgccgtggtacgcacagggctattttgtttacgactccaaaaaggcgggcggcctgacggtttctcaccttcgagtgagcgaacagccgattcgttccgcttatctcatttcccaggctgattttgttggctgccaccagttgcagtttatcgataaatatcagatggctgagcgtttaaaacctggcggcattttcctgctcaacacaccgtacagcgcagatgaagtgtggtcgcgcttgccgcaagaagttcaggccatgttaaaccagaaaaaagcgcgcttctatgtgattaacgcggcgaaaatcgcccgcgaatgtggcctggcggcccgtattaataccgtcatgcagatggcttttttccatctgacgcaaattctgcctggcgatagcgccctcgcagaattgcagggtgcgattgccaaaagttacagtagcaaaggccaggatctggtggaacgcaactggcaggctctggcgctggcgcgtgaatccgtagaagaagttccgttgcaaccggtaaatccgcacagcgccaatcgaccgcaagtggtttccgatgccgcccctgatttcgtgaaaaccgtaaccgctgcgatgctcgccgggcttggtgacgccctccccgtttcggcgctgccgccagacggcacctggccgatgggcactacgcgctgggaaaaacgcaatatcgccgaagagatccccatctggaaagaggaactctgtacccaatgtaaccactgcgttgccgcttgcccacactcagctattcgcgcaaaagtggtgccgcctgaagcgatggaaaacgcccctgccagcctgcattcgctggatgtgaaatcgcgtgatatgcgcgggcagaaatatgtcttgcaggtggcaccggaagattgcaccggttgtaacctgtgcgtcgaagtttgcccggcgaaagaccgtcagaatccagagattaaagccatcaatatgatgtctcgcctggaacatgtcgaagaagagaaaatcaattacgatttcttcctcaacctgccagaaatcgaccgtagcaaactggaacgtattgatattcgtacatcgcagctgattacaccgctgtttgaatattcaggtgcttgctccggttgtggcgagacgccgtatattaaattactgactcagctctatggcgaccggatgttgatcgctaacgccactggctgttcttcaatttatggcggtaacctgccctctacaccgtataccaccgatgccaacggtcgtgggccggcatgggcgaactctctatttgaagataatgccgaatttggccttggtttccgcctgacggtcgatcaacaccgtgtccgcgtgctgcgtctgctggatcaatttgccgataaaatcccggcggaattactgacggcgttgaaatcagacgccacgccagaggttcgtcgtgaacaggttgcagctttacgccagcaactcaacgatgttgccgaagcacatgaactgctacgtgatgcagatgcactggtggaaaaatcaatctggctgattggtggtgatggctgggcttacgatatcggctttggcggtctggatcatgtattgagtttgacggaaaacgtcaacattctggtgctggatacgcaatgctattccaacaccggtggtcaggcgtcgaaagcgacaccgctgggtgcagtaactaaatttggcgagcacggcaaacgtaaagcgcgtaaagatcttggcgtcagtatgatgatgtacggtcatgtttatgtggcgcagatttctctcggcgcgcagctgaaccagacggtgaaagcgattcaggaagcggaagcgtatccggggccatcgctgatcattgcttatagcccgtgtgaagagcatggttacgatctggcactcagccacgaccagatgcgccaactcacagctaccggcttctggccgctatatcgctttgatccgcgtcgtgccgatgaaggcaaactgccgctggccttggattcacgcccgccgtcagaagcactggaagaaacgttacttcacgagcaacgtttccgtcggctgaattcgcagcagccagaagtggcagaacagttatggaaagatgctgcagctgatttgcaaaaacgctatgacttcctggcacaaatggccggaaaagcggaaaaaagcaacaccgattaa

SEQ ID NO: 36 - protein, 1174 aa, Escherichia coli
MITIDGNGAVASVAFRTSEVIAIYPITPSSTMAEQADAWAGNGLKNVWGDTPRVVEMQSEAGAIATVHGALQTGALSTSFTSSQGLLLMIPTLYKLAGELTPFVLHVAARTVATHALSIFGDHSDVMAVRQTGCAMLCAANVQEAQDFALISHIATLKSRVPFIHFFDGFRTSHEINKIVPLADDTILDLMPQAEIDAHRARALNPEHPVIRGTSANPDTYFQSREATNPWYNAVYDHVEQAMNDFAAATGRQYQPFEYYGHPQAERVIILMGSAIGTCEEVIDELLTRGENVGVLKVRLYRPFSAKHLLQALPGSVRSVAVLDRTKEPGAQAEPLYLDVMTALAEAFNNGERETLPRVIGGRYGLSSKEFGPDCVLAVFAELNAAKPKARFTVGIYDDVTNLSLPLPENTLPNSAKLEALFYGLGSDGSVSATKNNIKIIGNSTPWYAQGYFVYDSKKAGGLTVSHLRVSEQPIRSAYLISQADFVGCHQLQFIDKYQMAERLKPGGIFLLNTPYSADEVWSRLPQEVQAMLNQKKARFYVINAAKIARECGLAARINTVMQMAFFHLTQILPGDSALAELQGAIAKSYSSKGQDLVERNWQALALARESVEEVPLQPVNPHSANRPQVVSDAAPDFVKTVTAAMLAGLGDALPVSALPPDGTWPMGTTRWEKRNIAEEIPIWKEELCTQCNHCVAACPHSAIRAKVVPPEAMENAPASLHSLDVKSRDMRGQKYVLQVAPEDCTGCNLCVEVCPAKDRQNPEIKAINMMSRLEHVEEEKINYDFFLNLPEIDRSKLERIDIRTSQLITPLFEYSGACSGCGETPYIKLLTQLYGDRMLIANATGCSSIYGGNLPSTPYTTDANGRGPAWANSLFEDNAEFGLGFRLTVDQHRVRVLRLLDQFADKIPAELLTALKSDATPEVRREQVAALRQQLNDVAEAHELLRDADALVEKSIWLIGGDGWAYDIGFGGLDHVLSLTENVNILVLDTQCYSNTGGQASKATPLGAVTKFGEHGKRKARKDLGVSMMMYGHVYVAQISLGAQLNQTVKAIQEAEAYPGPSLIIAYSPCEEHGYDLALSHDQMRQLTATGFWPLYRFDPRRADEGKLPLALDSRPPSEALEETLLHEQRFRRLNSQQPEVAEQLWKDAAADLQKRYDFLAQMAGKAEKSNTD

SEQ ID NO: 37 - DNA, 747 bp, Escherichia coli
atggctgattgggtaacaggcaaagtcactaaagtgcagaactggaccgacgccctgtttagtctcaccgttcacgcccccgtgcttccgtttaccgccgggcaatttaccaagcttggccttgaaatcgacggcgaacgcgtccagcgcgcctactcctatgtaaactcgcccgataatcccgatctggagttttacctggtcaccgtccccgatggcaaattaagcccacgactggcggcactgaaaccaggcgatgaagtgcaggtggttagcgaagcggcaggattctttgtgctcgatgaagtgccgcactgcgaaacgctatggatgctggcaaccggtacagcgattggcccttatttatcgattctgcaactaggtaaagatttagatcgcttcaaaaatctggtcctggtgcacgccgcacgttatgccgccgacttaagctatttgccactgatgcaggaactggaaaaacgctacgaaggaaaactgcgcattcagacggtggtcagtcgggaaacggcagcggggtcgctcaccggacggataccggcattaattgaaagtggggaactggaaagcacgattggcctgccgatgaataaagaaaccagccatgtgatgctgtgcggcaatccacagatggtgcgcgatacacaacagttgctgaaagagacccggcagatgacgaaacatttacgtcgccgaccgggccacatgacagcggagcattactggtaa

SEQ ID NO: 38 - protein, 248 aa, Escherichia coli
MADWVTGKVTKVQNWTDALFSLTVHAPVLPFTAGQFTKLGLEIDGERVQRAYSYVNSPDNPDLEFYLVTVPDGKLSPRLAALKPGDEVQVVSEAAGFFVLDEVPHCETLWMLATGTAIGPYLSILQLGKDLDRFKNLVLVHAARYAADLSYLPLMQELEKRYEGKLRIQTVVSRETAAGSLTGRIPALIESGELESTIGLPMNKETSHVMLCGNPQMVRDTQQLLKETRQMTKHLRRRPGHMTAEHYW

SEQ ID NO: 39 - DNA, 336 bp, Escherichia coli
atgccaaagattgttattttgcctcatcaggatctctgccctgatggcgctgttctggaagctaatagcggtgaaaccattctcgacgcagctctgcgtaacggtatcgagattgaacacgcctgtgaaaaatcctgtgcttgcaccacctgccactgcatcgttcgtgaaggttttgactcactgccggaaagctcagagcaggaagacgacatgctggacaaagcctggggactggagccggaaagccgtttaagctgccaggcgcgcgttaccgacgaagatttagtagtcgaaatcccgcgttacactatcaaccatgcgcgtgagcattaa

SEQ ID NO: 40 - protein, 111 aa, Escherichia coli
MPKIVILPHQDLCPDGAVLEANSGETILDAALRNGIEIEHACEKSCACTTCHCIVREGFDSLPESSEQEDDMLDKAWGLEPESRLSCQARVTDEDLVVEIPRYTINHAREH

SEQ ID NO: 41 - DNA, 531 bp, Escherichia coli
atggctatcactggcatctttttcggcagcgacaccggtaataccgaaaatatcgcaaaaatgattcaaaaacagcttggtaaagacgttgccgatgtccatgacattgcaaaaagcagcaaagaagatctggaagcttatgacattctgctgctgggcatcccaacctggtattacggcgaagcgcagtgtgactgggatgacttcttcccgactctcgaagagattgatttcaacggcaaactggttgcgctgtttggttgtggtgaccaggaagattacgccgaatatttctgcgacgcattgggcaccatccgcgacatcattgaaccgcgcggtgcaaccatcgttggtcactggccaactgcgggctatcatttcgaagcatcaaaaggtctggcagatgacgaccactttgtcggtctggctatcgacgaagaccgtcagccggaactgaccgctgaacgtgtagaaaaatgggttaaacagatttctgaagagttgcatctcgacgaaattctcaatgcctga

SEQ ID NO: 42 - protein, 176 aa, Escherichia coli
MAITGIFFGSDTGNTENIAKMIQKQLGKDVADVHDIAKSSKEDLEAYDILLLGIPTWYYGEAQCDWDDFFPTLEEIDFNGKLVALFGCGDQEDYAEYFCDALGTIRDIIEPRGATIVGHWPTAGYHFEASKGLADDDHFVGLAIDEDRQPELTAERVEKWVKQISEELHLDEILNA

SEQ ID NO: 43 - DNA, 522 bp, Escherichia coli
atgaatatgggtcttttttacggttccagcacctgttacaccgaaatggcggcagaaaaaatccgcgatattatcggcccagaactggtgaccttacataacctcaaggacgactccccgaaattaatggagcagtacgatgtgctcattctgggtatcccgacctgggattttggtgaaatccaggaagactgggaagccgtctgggatcagctcgacgacctgaaccttgaaggtaaaattgttgcgctgtatgggcttggcgatcaactgggatacggcgagtggttcctcgatgcgctcggtatgctgcatgacaaactctcgaccaaaggcgtgaagttcgtcggctactggccaacggaaggatatgaatttaccagcccgaaaccggtgattgctgacgggcaactgttcgtgggtctggcgctggatgaaactaaccagtatgaccttagcgacgagcgtattcagagctggtgcgagcaaatcctcaacgaaatggcagagcattacgcctga

SEQ ID NO: 44 - protein, 173 aa, Escherichia coli
MNMGLFYGSSTCYTEMAAEKIRDIIGPELVTLHNLKDDSPKLMEQYDVLILGIPTWDFGEIQEDWEAVWDQLDDLNLEGKIVALYGLGDQLGYGEWFLDALGMLHDKLSTKGVKFVGYWPTEGYEFTSPKPVIADGQLFVGLALDETNQYDLSDERIQSWCEQILNEMAEHYA

SEQ ID NO: 45 - DNA, 34 bp, Artificial Sequence
  Synthetic Construct
tagccatcggtggggccaggtgagtcacagcggt

SEQ ID NO: 46 - DNA, 38 bp, Artificial Sequence
  Synthetic Construct
ggccccaccgatggctatgtttaccactaccgcgaaag

SEQ ID NO: 47 - DNA, 42 bp, Artificial Sequence
  Synthetic Construct
cacggattttcggctggataactttcgcggtagtggtaaaca

SEQ ID NO: 48 - DNA, 45 bp, Artificial Sequence
  Synthetic Construct
ttatccagccgaaaatccgtggttttatctgcactaccactcacc

SEQ ID NO: 49 - DNA, 44 bp, Artificial Sequence
  Synthetic Construct
gcgtttttcgcagccaattgggtgagtggtagtgcagataaaac

SEQ ID NO: 50 - DNA, 44 bp, Artificial Sequence
  Synthetic Construct
caattggctgcgaaaaacgcgtccaggaagaaattgcttacgct

SEQ ID NO: 51 - DNA, 40 bp, Artificial Sequence
  Synthetic Construct
ttggcgggtgcgcacgagcgtaagcaatttcttcctggac

SEQ ID NO: 52 - DNA, 32 bp, Artificial Sequence
  Synthetic Construct
cgtgcgcacccgccaaccagccctggccctaa

SEQ ID NO: 53 - DNA, 34 bp, Artificial Sequence
  Synthetic Construct
ccgatgaccagtacacgcttagggccagggctgg

SEQ ID NO: 54 - DNA, 37 bp, Artificial Sequence
  Synthetic Construct
gcgtgtactggtcatcggttgtagcacgggttacggt

SEQ ID NO: 55 - DNA, 40 bp, Artificial Sequence
  Synthetic Construct
agcagtgatacgggtagacagaccgtaacccgtgctacaa

SEQ ID NO: 56 - DNA, 38 bp, Artificial Sequence
  Synthetic Construct
ctgtctacccgtatcactgctgcgttcggctaccaggc

SEQ ID NO: 57 - DNA, 33 bp, Artificial Sequence
  Synthetic Construct
aacgcccagggtcgccgcctggtagccgaacgc

SEQ ID NO: 58 - DNA, 32 bp, Artificial Sequence
  Synthetic Construct
ggcgaccctgggcgttttcctggcgggtccac

SEQ ID NO: 59 - DNA, 32 bp, Artificial Sequence
  Synthetic Construct
gggcgacctttggtcggtggacccgccaggaa

SEQ ID NO: 60 - DNA, 32 bp, Artificial Sequence
  Synthetic Construct
cgaccaaaggtcgcccggcagctgcgggttgg

SEQ ID NO: 61 - DNA, 37 bp, Artificial Sequence
  Synthetic Construct
ctcgaaggcaacagtgttgtaccaacccgcagctgcc

SEQ ID NO: 62 - DNA, 37 bp, Artificial Sequence
  Synthetic Construct
tacaacactgttgccttcgagaaagcagcgctggagg

SEQ ID NO: 63 - DNA, 32 bp, Artificial Sequence
  Synthetic Construct
cgggcatacaggcccgcctccagcgctgcttt

SEQ ID NO: 64 - DNA, 34 bp, Artificial Sequence
  Synthetic Construct
cgggcctgtatgcccgttctctgaacggcgacgc

SEQ ID NO: 65 - DNA, 41 bp, Artificial Sequence
  Synthetic Construct
gcgctttcgtagtggaatcaaaagcgtcgccgttcagagaa

SEQ ID NO: 66 - DNA, 45 bp, Artificial Sequence
  Synthetic Construct
ttttgattccactacgaaagcgcgcactgttgaagctatcaaacg

SEQ ID NO: 67 - DNA, 38 bp, Artificial Sequence
  Synthetic Construct
tacggtgcccaggtcacgtttgatagcttcaacagtgc

SEQ ID NO: 68 - DNA, 39 bp, Artificial Sequence
  Synthetic Construct
tgacctgggcaccgtagacctggtagtgtactctatcgc

SEQ ID NO: 69 - DNA, 39 bp, Artificial Sequence
  Synthetic Construct
ggtacgcttcggggcagcgatagagtacactaccaggtc

SEQ ID NO: 70 - DNA, 32 bp, Artificial Sequence
  Synthetic Construct
tgccccgaagcgtaccgatccggcgaccggcg

SEQ ID NO: 71 - DNA, 35 bp, Artificial Sequence
  Synthetic Construct
agacaagccttgtgcagaacgccggtcgccggatc

SEQ ID NO: 72 - DNA, 36 bp, Artificial Sequence
  Synthetic Construct
ttctgcacaaggcttgtctgaaaccaatcggcgcga

SEQ ID NO: 73 - DNA, 37 bp, Artificial Sequence
  Synthetic Construct
gacggtacggttggtgtaagtcgcgccgattggtttc

SEQ ID NO: 74 - DNA, 38 bp, Artificial Sequence
  Synthetic Construct
cttacaccaaccgtaccgtcaacaccgacaaagcggag

SEQ ID NO: 75 - DNA, 38 bp, Artificial Sequence
  Synthetic Construct
tcgatgctaacatcggtcacctccgctttgtcggtgtt

SEQ ID NO: 76 - DNA, 36 bp, Artificial Sequence
  Synthetic Construct
gtgaccgatgttagcatcgaacctgcctccccggaa

SEQ ID NO: 77 - DNA, 33 bp, Artificial Sequence
  Synthetic Construct
accgtgtccgcgatctcttccggggaggcaggt

SEQ ID NO: 78 - DNA, 39 bp, Artificial Sequence
  Synthetic Construct
gagatcgcggacacggttaaagtgatgggtggtgaagac

SEQ ID NO: 79 - DNA, 41 bp, Artificial Sequence
  Synthetic Construct
cctgaatccacagctcccagtcttcaccacccatcacttta

SEQ ID NO: 80 - DNA, 35 bp, Artificial Sequence
  Synthetic Construct
tgggagctgtggattcaggcgctgagcgaagccgg

SEQ ID NO: 81 - DNA, 32 bp, Artificial Sequence
  Synthetic Construct
accctccgccagaacaccggcttcgctcagcg

SEQ ID NO: 82 - DNA, 33 bp, Artificial Sequence
  Synthetic Construct
tgttctggcggagggtgcgaaaaccgtggcgta

SEQ ID NO: 83 - DNA, 37 bp, Artificial Sequence
  Synthetic Construct
tctcagggccaatgtaggagtacgccacggttttcgc

SEQ ID NO: 84 - DNA, 40 bp, Artificial Sequence
  Synthetic Construct
ctcctacattggccctgagatgacctggccggtatattgg

SEQ ID NO: 85 - DNA, 41 bp, Artificial Sequence
  Synthetic Construct
ggcttcgccaatagtaccagaccaatataccggccaggtca

SEQ ID NO: 86 - DNA, 43 bp, Artificial Sequence
  Synthetic Construct
tctggtactattggcgaagccaaaaaggatgttgaaaaggcgg

SEQ ID NO: 87 - DNA, 43 bp, Artificial Sequence
  Synthetic Construct
actgctgggtgatacgtttagccgccttttcaacatccttttt

SEQ ID NO: 88 - DNA, 39 bp, Artificial Sequence
  Synthetic Construct
ctaaacgtatcacccagcagtatggttgcccagcatacc

SEQ ID NO: 89 - DNA, 36 bp, Artificial Sequence
  Synthetic Construct
gcgctttagcgactaccgggtatgctgggcaaccat

SEQ ID NO: 90 - DNA, 34 bp, Artificial Sequence
  Synthetic Construct
cggtagtcgctaaagcgctggtcacccaggccag

SEQ ID NO: 91 - DNA, 35 bp, Artificial Sequence
  Synthetic Construct
aactaccggaattgcggagctggcctgggtgacca

SEQ ID NO: 92 - DNA, 39 bp, Artificial Sequence
  Synthetic Construct
ctccgcaattccggtagttccactgtacatttgcctgct

SEQ ID NO: 93 - DNA, 44 bp, Artificial Sequence
  Synthetic Construct
cctttttctttcatcacacggtacagcaggcaaatgtacagtgg

SEQ ID NO: 94 - DNA, 48 bp, Artificial Sequence
  Synthetic Construct
gtaccgtgtgatgaaagaaaaaggtactcatgaaggttgcattgaaca

SEQ ID NO: 95 - DNA, 43 bp, Artificial Sequence
  Synthetic Construct
ggtcagcagacgaaccatctgttcaatgcaaccttcatgagta

SEQ ID NO: 96 - DNA, 42 bp, Artificial Sequence
  Synthetic Construct
gatggttcgtctgctgaccactaaactgtaccctgagaacgg

SEQ ID NO: 97 - DNA, 40 bp, Artificial Sequence
  Synthetic Construct
cgtccacgatcggagcaccgttctcagggtacagtttagt

SEQ ID NO: 98 - DNA, 33 bp, Artificial Sequence
  Synthetic Construct
tgctccgatcgtggacgaagcgggccgtgttcg

SEQ ID NO: 99 - DNA, 37 bp, Artificial Sequence
  Synthetic Construct
gccatttcccagtcatcaacacgaacacggcccgctt

SEQ ID NO: 100 - DNA, 39 bp, Artificial Sequence
  Synthetic Construct
tgttgatgactgggaaatggctgaagacgtgcagcaagc

SEQ ID NO: 101 - DNA, 39 bp, Artificial Sequence
  Synthetic Construct
tgggaccacaggtctttaacagcttgctgcacgtcttca

SEQ ID NO: 102 - DNA, 41 bp, Artificial Sequence
  Synthetic Construct
tgttaaagacctgtggtcccaggtgtctacggctaacctga

SEQ ID NO: 103 - DNA, 39 bp, Artificial Sequence
  Synthetic Construct
gcgaagtcgctgatgtctttcaggttagccgtagacacc

SEQ ID NO: 104 - DNA, 39 bp, Artificial Sequence
  Synthetic Construct
aagacatcagcgacttcgctggctaccaaactgagttcc

SEQ ID NO: 105 - DNA, 41 bp, Artificial Sequence
  Synthetic Construct
taccaaaaccaaacagacgcaggaactcagtttggtagcca

SEQ ID NO: 106 - DNA, 41 bp, Artificial Sequence
  Synthetic Construct
tgcgtctgtttggttttggtatcgacggtgtagactacgac

SEQ ID NO: 107 - DNA, 37 bp, Artificial Sequence
  Synthetic Construct
tcaacgtcaaccggctggtcgtagtctacaccgtcga

SEQ ID NO: 108 - DNA, 33 bp, Artificial Sequence
  Synthetic Construct
cagccggttgacgttgaagcggacctgccgagc

SEQ ID NO: 109 - DNA, 36 bp, Artificial Sequence
  Synthetic Construct
aattcttattgctgcgctgcgctcggcaggtccgct

SEQ ID NO: 110 - DNA, 39 bp, Artificial Sequence
  Synthetic Construct
gcagcgcagcaataagaattcctcgagtcatgacaggcc

SEQ ID NO: 111 - DNA, 35 bp, Artificial Sequence
  Synthetic Construct
taccgcgaggggttgcggcctgtcatgactcgagg

SEQ ID NO: 112 - DNA, 16 bp, Artificial Sequence
  Synthetic Construct
gcaacccctcgcggta

SEQ ID NO: 113 - DNA, 16 bp, Artificial Sequence
  Synthetic Construct
tcgtggcttgctggac

SEQ ID NO: 114 - DNA, 39 bp, Artificial Sequence
  Synthetic Construct
gtccagcaagccacgagatatacatatgatcgtcaagcc

SEQ ID NO: 115 - DNA, 43 bp, Artificial Sequence
  Synthetic Construct
agatattattgcgcaccattggcttgacgatcatatgtatatc

SEQ ID NO: 116 - DNA, 36 bp, Artificial Sequence
  Synthetic Construct
aatggtgcgcaataatatctgtctgaacgctcaccc

SEQ ID NO: 117 - DNA, 34 bp, Artificial Sequence
  Synthetic Construct
ccctttttacaaccctgcgggtgagcgttcagac

SEQ ID NO: 118 - DNA, 41 bp, Artificial Sequence
  Synthetic Construct
gcagggttgtaaaaagggtgtagaagaccagattgaataca

SEQ ID NO: 119 - DNA, 41 bp, Artificial Sequence
  Synthetic Construct
cggtgatgcgtttcttagtgtattcaatctggtcttctaca

SEQ ID NO: 120 - DNA, 37 bp, Artificial Sequence
  Synthetic Construct
ctaagaaacgcatcaccgcagaagttaaagcaggtgc

SEQ ID NO: 121 - DNA, 37 bp, Artificial Sequence
  Synthetic Construct
acgtttttcggtgctttggcacctgctttaacttctg

SEQ ID NO: 122 - DNA, 33 bp, Artificial Sequence
  Synthetic Construct
caaagcaccgaaaaacgtcctggtgctgggctg

SEQ ID NO: 123 - DNA, 30 bp, Artificial Sequence
  Synthetic Construct
ccgtagccgttgctgcagcccagcaccagg

SEQ ID NO: 124 - DNA, 30 bp, Artificial Sequence
  Synthetic Construct
cagcaacggctacggtctggcaagccgcat

SEQ ID NO: 125 - DNA, 30 bp, Artificial Sequence
  Synthetic Construct
ccgaatgcagccgtaatgcggcttgccaga

SEQ ID NO: 126 - DNA, 30 bp, Artificial Sequence
  Synthetic Construct
tacggctgcattcggttacggcgctgctac

SEQ ID NO: 127 - DNA, 36 bp, Artificial Sequence
  Synthetic Construct
ttttcgaagctaacaccaatagtagcagcgccgtaa

SEQ ID NO: 128 - DNA, 37 bp, Artificial Sequence
  Synthetic Construct
tattggtgttagcttcgaaaaggcgggttctgaaacc

SEQ ID NO: 129 - DNA, 33 bp, Artificial Sequence
  Synthetic Construct
cctggagtgccgtatttggtttcagaacccgcc

SEQ ID NO: 130 - DNA, 35 bp, Artificial Sequence
  Synthetic Construct
aaatacggcactccaggctggtacaacaacctggc

SEQ ID NO: 131 - DNA, 34 bp, Artificial Sequence
  Synthetic Construct
cgctgcttcgtcgaatgccaggttgttgtaccag

SEQ ID NO: 132 - DNA, 33 bp, Artificial Sequence
  Synthetic Construct
attcgacgaagcagcgaagcgtgagggtctgta

SEQ ID NO: 133 - DNA, 35 bp, Artificial Sequence
  Synthetic Construct
ccgtcgatggtaacagagtacagaccctcacgctt

SEQ ID NO: 134 - DNA, 34 bp, Artificial Sequence
  Synthetic Construct
ctctgttaccatcgacggtgacgcgttctctgac

SEQ ID NO: 135 - DNA, 37 bp, Artificial Sequence
  Synthetic Construct
gataacctgagctttgatctcgtcagagaacgcgtca

SEQ ID NO: 136 - DNA, 43 bp, Artificial Sequence
  Synthetic Construct
gagatcaaagctcaggttatcgaggaagctaaaaagaaaggta

SEQ ID NO: 137 - DNA, 42 bp, Artificial Sequence
  Synthetic Construct
cacaatcaggtcgaatttgatacctttctttttagcttcctc

SEQ ID NO: 138 - DNA, 36 bp, Artificial Sequence
  Synthetic Construct
tcaaattcgacctgattgtgtactccctggcctctc

SEQ ID NO: 139 - DNA, 31 bp, Artificial Sequence
  Synthetic Construct
ggtcggtacgaaccggagaggccagggagta

SEQ ID NO: 140 - DNA, 31 bp, Artificial Sequence
  Synthetic Construct
cggttcgtaccgacccggataccggcatcat

SEQ ID NO: 141 - DNA, 34 bp, Artificial Sequence
  Synthetic Construct
ttcagtacgcttttgtgcatgatgccggtatccg

SEQ ID NO: 142 - DNA, 34 bp, Artificial Sequence
  Synthetic Construct
gcacaaaagcgtactgaagccgtttggcaaaacc

SEQ ID NO: 143 - DNA, 35 bp, Artificial Sequence
  Synthetic Construct
caacggttttaccagtgaaggttttgccaaacggc

SEQ ID NO: 144 - DNA, 34 bp, Artificial Sequence
  Synthetic Construct
ttcactggtaaaaccgttgatcctttcaccggcg

SEQ ID NO: 145 - DNA, 33 bp, Artificial Sequence
  Synthetic Construct
cggagatttccttcagctcgccggtgaaaggat

SEQ ID NO: 146 - DNA, 33 bp, Artificial Sequence
  Synthetic Construct
agctgaaggaaatctccgccgagccagctaacg

SEQ ID NO: 147 - DNA, 31 bp, Artificial Sequence
  Synthetic Construct
gcagcagcctcctcatcgttagctggctcgg

SEQ ID NO: 148 - DNA, 35 bp, Artificial Sequence
  Synthetic Construct
atgaggaggctgctgcgaccgttaaagtgatgggt

SEQ ID NO: 149 - DNA, 35 bp, Artificial Sequence
  Synthetic Construct
gttcccagtcttcgccacccatcactttaacggtc

SEQ ID NO: 150 - DNA, 36 bp, Artificial Sequence
  Synthetic Construct
ggcgaagactgggaacgttggatcaaacaactgtcc

SEQ ID NO: 151 - DNA, 36 bp, Artificial Sequence
  Synthetic Construct
ccagcagaccttccttggacagttgtttgatccaac

SEQ ID NO: 152 - DNA, 35 bp, Artificial Sequence
  Synthetic Construct
aaggaaggtctgctggaggagggctgtattactct

SEQ ID NO: 153 - DNA, 38 bp, Artificial Sequence
  Synthetic Construct
gccgatgtaagaatatgccagagtaatacagccctcct

SEQ ID NO: 154 - DNA, 34 bp, Artificial Sequence
  Synthetic Construct
ggcatattcttacatcggcccggaggcgactcag

SEQ ID NO: 155 - DNA, 33 bp, Artificial Sequence
  Synthetic Construct
gcccttacgatacagtgcctgagtcgcctccgg

SEQ ID NO: 156 - DNA, 35 bp, Artificial Sequence
  Synthetic Construct
gcactgtatcgtaagggcaccatcggtaaagcgaa

SEQ ID NO: 157 - DNA, 34 bp, Artificial Sequence
  Synthetic Construct
tggcctccagatgttctttcgctttaccgatggt

SEQ ID NO: 158 - DNA, 32 bp, Artificial Sequence
  Synthetic Construct
agaacatctggaggccaccgctcaccgtctga

SEQ ID NO: 159 - DNA, 32 bp, Artificial Sequence
  Synthetic Construct
gctcgggttttccttgttcagacggtgagcgg

SEQ ID NO: 160 - DNA, 33 bp, Artificial Sequence
  Synthetic Construct
acaaggaaaacccgagcatccgtgctttcgtgt

SEQ ID NO: 161 - DNA, 32 bp, Artificial Sequence
  Synthetic Construct
aggcccttgttaacggacacgaaagcacggat

SEQ ID NO: 162 - DNA, 31 bp, Artificial Sequence
  Synthetic Construct
ccgttaacaagggcctggttacgcgcgcttc

SEQ ID NO: 163 - DNA, 32 bp, Artificial Sequence
  Synthetic Construct
tgaccggaattactgcggaagcgcgcgtaacc

SEQ ID NO: 164 - DNA, 33 bp, Artificial Sequence
  Synthetic Construct
cgcagtaattccggtcattccgctgtacctggc

SEQ ID NO: 165 - DNA, 37 bp, Artificial Sequence
  Synthetic Construct
ttcatgactttaaacagggaagccaggtacagcggaa

SEQ ID NO: 166 - DNA, 38 bp, Artificial Sequence
  Synthetic Construct
ttccctgtttaaagtcatgaaagaaaaaggcaaccacg

SEQ ID NO: 167 - DNA, 40 bp, Artificial Sequence
  Synthetic Construct
agtaatttgttcgatacaaccttcgtggttgcctttttct

SEQ ID NO: 168 - DNA, 38 bp, Artificial Sequence
  Synthetic Construct
aaggttgtatcgaacaaattactcgcctgtatgcggag

SEQ ID NO: 169 - DNA, 31 bp, Artificial Sequence
  Synthetic Construct
ccttacggtacaggcgctccgcatacaggcg

SEQ ID NO: 170 - DNA, 32 bp, Artificial Sequence
  Synthetic Construct
cgcctgtaccgtaaggatggcactatcccggt

SEQ ID NO: 171 - DNA, 33 bp, Artificial Sequence
  Synthetic Construct
tgcggttctcttcatcaaccgggatagtgccat

SEQ ID NO: 172 - DNA, 34 bp, Artificial Sequence
  Synthetic Construct
tgatgaagagaaccgcatccgcattgacgattgg

SEQ ID NO: 173 - DNA, 37 bp, Artificial Sequence
  Synthetic Construct
tgtacatcctcttccagttcccaatcgtcaatgcgga

SEQ ID NO: 174 - DNA, 35 bp, Artificial Sequence
  Synthetic Construct
gaactggaagaggatgtacagaaagcggtttccgc

SEQ ID NO: 175 - DNA, 34 bp, Artificial Sequence
  Synthetic Construct
cgtcactttttccatcagcgcggaaaccgctttc

SEQ ID NO: 176 - DNA, 34 bp, Artificial Sequence
  Synthetic Construct
gctgatggaaaaagtgacgggcgaaaacgcggaa

SEQ ID NO: 177 - DNA, 31 bp, Artificial Sequence
  Synthetic Construct
ccagatccgtcagggattccgcgttttcgcc

SEQ ID NO: 178 - DNA, 32 bp, Artificial Sequence
  Synthetic Construct
tccctgacggatctggcaggttaccgtcacga

SEQ ID NO: 179 - DNA, 35 bp, Artificial Sequence
  Synthetic Construct
accattagacgccagaaagtcgtgacggtaacctg

SEQ ID NO: 180 - DNA, 38 bp, Artificial Sequence
  Synthetic Construct
ctttctggcgtctaatggtttcgacgttgagggtatta

SEQ ID NO: 181 - DNA, 37 bp, Artificial Sequence
  Synthetic Construct
caacttctgcctcgtagttaataccctcaacgtcgaa

SEQ ID NO: 182 - DNA, 41 bp, Artificial Sequence
  Synthetic Construct
actacgaggcagaagttgaacgtttcgatcgtatttaatct

SEQ ID NO: 183 - DNA, 38 bp, Artificial Sequence
  Synthetic Construct
atgccctggcgttctagattaaatacgatcgaaacgtt

SEQ ID NO: 184 - DNA, 15 bp, Artificial Sequence
  Synthetic Construct
agaacgccagggcat

SEQ ID NO: 185 - DNA, 22 bp, Artificial Sequence
  Synthetic Construct
agcgtccaggatttctttcatc

SEQ ID NO: 186 - DNA, 40 bp, Artificial Sequence
  Synthetic Construct
gatgaaagaaatcctggacgctattcaggcacaaacggca

SEQ ID NO: 187 - DNA, 34 bp, Artificial Sequence
  Synthetic Construct
ccgtaccgcttgcggttgccgtttgtgcctgaat

SEQ ID NO: 188 - DNA, 33 bp, Artificial Sequence
  Synthetic Construct
accgcaagcggtacggcggctgttacctctgct

SEQ ID NO: 189 - DNA, 33 bp, Artificial Sequence
  Synthetic Construct
gcagggccgcaaaatcagcagaggtaacagccg

SEQ ID NO: 190 - DNA, 33 bp, Artificial Sequence
  Synthetic Construct
gattttgcggccctgccgctgccggactcttac

SEQ ID NO: 191 - DNA, 35 bp, Artificial Sequence
  Synthetic Construct
gtgaaccgtgatcgcacggtaagagtccggcagcg

SEQ ID NO: 192 - DNA, 41 bp, Artificial Sequence
  Synthetic Construct
cgtgcgatcacggttcacaaggatgaaaccgagatgttcgc

SEQ ID NO: 193 - DNA, 43 bp, Artificial Sequence
  Synthetic Construct
tatcacgggattccagaccggcgaacatctcggtttcatcctt

SEQ ID NO: 194 - DNA, 42 bp, Artificial Sequence
  Synthetic Construct
cggtctggaatcccgtgataaagatcctcgtaaatctctgca

SEQ ID NO: 195 - DNA, 38 bp, Artificial Sequence
  Synthetic Construct
tggcacgtcatccaggtgcagagatttacgaggatctt

SEQ ID NO: 196 - DNA, 33 bp, Artificial Sequence
  Synthetic Construct
cctggatgacgtgccaattccggaactgggtcc

SEQ ID NO: 197 - DNA, 33 bp, Artificial Sequence
  Synthetic Construct
aaccagcgcttcgcccggacccagttccggaat

SEQ ID NO: 198 - DNA, 34 bp, Artificial Sequence
  Synthetic Construct
gggcgaagcgctggttgctgtgatggcttcctct

SEQ ID NO: 199 - DNA, 41 bp, Artificial Sequence
  Synthetic Construct
gtccagacgctgttataattcacagaggaagccatcacagc

SEQ ID NO: 200 - DNA, 41 bp, Artificial Sequence
  Synthetic Construct
gtgaattataacagcgtctggacttccatcttcgaaccggt

SEQ ID NO: 201 - DNA, 38 bp, Artificial Sequence
  Synthetic Construct
ccaggaagctgaaggtagataccggttcgaagatggaa

SEQ ID NO: 202 - DNA, 38 bp, Artificial Sequence
  Synthetic Construct
atctaccttcagcttcctggaacgttacggtcgtctgt

SEQ ID NO: 203 - DNA, 37 bp, Artificial Sequence
  Synthetic Construct
tgacgtttgctcagatcggacagacgaccgtaacgtt

SEQ ID NO: 204 - DNA, 36 bp, Artificial Sequence
  Synthetic Construct
ccgatctgagcaaacgtcatgacctgccgtaccaca

SEQ ID NO: 205 - DNA, 36 bp, Artificial Sequence
  Synthetic Construct
gccagatcagaaccgatgatgtggtacggcaggtca

SEQ ID NO: 206 - DNA, 36 bp, Artificial Sequence
  Synthetic Construct
tcatcggttctgatctggccggtgtcgttctgcgta

SEQ ID NO: 207 - DNA, 34 bp, Artificial Sequence
  Synthetic Construct
gttgacaccaggaccggtacgcagaacgacaccg

SEQ ID NO: 208 - DNA, 34 bp, Artificial Sequence
  Synthetic Construct
ccggtcctggtgtcaacgcttggaacccaggtga

SEQ ID NO: 209 - DNA, 33 bp, Artificial Sequence
  Synthetic Construct
atgcgccaccacttcgtcacctgggttccaagc

SEQ ID NO: 210 - DNA, 32 bp, Artificial Sequence
  Synthetic Construct
cgaagtggtggcgcattgcctgagcgtggaac

SEQ ID NO: 211 - DNA, 33 bp, Artificial Sequence
  Synthetic Construct
gccatcggaggactccagttccacgctcaggca

SEQ ID NO: 212 - DNA, 39 bp, Artificial Sequence
  Synthetic Construct
tggagtcctccgatggccacaacgatactatgctggacc

SEQ ID NO: 213 - DNA, 39 bp, Artificial Sequence
  Synthetic Construct
ccagatgcgctgttccgggtccagcatagtatcgttgtg

SEQ ID NO: 214 - DNA, 38 bp, Artificial Sequence
  Synthetic Construct
cggaacagcgcatctggggttttgaaaccaacttcggc

SEQ ID NO: 215 - DNA, 38 bp, Artificial Sequence
  Synthetic Construct
gcaatttcagccaggccgccgaagttggtttcaaaacc

SEQ ID NO: 216 - DNA, 38 bp, Artificial Sequence
  Synthetic Construct
ggcctggctgaaattgctctggtgaaatctaaccagct

SEQ ID NO: 217 - DNA, 39 bp, Artificial Sequence
  Synthetic Construct
tgacccggtttaggcatcagctggttagatttcaccaga

SEQ ID NO: 218 - DNA, 36 bp, Artificial Sequence
  Synthetic Construct
gatgcctaaaccgggtcatctgtcttgggaagaggc

SEQ ID NO: 219 - DNA, 34 bp, Artificial Sequence
  Synthetic Construct
cagacctgggctggcggcctcttcccaagacaga

SEQ ID NO: 220 - DNA, 40 bp, Artificial Sequence
  Synthetic Construct
cgccagcccaggtctggttaattctactgcataccgtcaa

SEQ ID NO: 221 - DNA, 43 bp, Artificial Sequence
  Synthetic Construct
cgccattacgggaaaccagttgacggtatgcagtagaattaac

SEQ ID NO: 222 - DNA, 37 bp, Artificial Sequence
  Synthetic Construct
ctggtttcccgtaatggcgcaggcatgaaacagggcg

SEQ ID NO: 223 - DNA, 38 bp, Artificial Sequence
  Synthetic Construct
accccaaatcagtacgttgtcgccctgtttcatgcctg

SEQ ID NO: 224 - DNA, 36 bp, Artificial Sequence
  Synthetic Construct
acaacgtactgatttggggtgcgtccggtggcctgg

SEQ ID NO: 225 - DNA, 39 bp, Artificial Sequence
  Synthetic Construct
cgcgaactgagtagcataagagcccaggccaccggacgc

SEQ ID NO: 226 - DNA, 39 bp, Artificial Sequence
  Synthetic Construct
gctcttatgctactcagttcgcgctggcgggtggtgcca

SEQ ID NO: 227 - DNA, 35 bp, Artificial Sequence
  Synthetic Construct
gaaacgacgcaaattgggttggcaccacccgccag

SEQ ID NO: 228 - DNA, 35 bp, Artificial Sequence
  Synthetic Construct
acccaatttgcgtcgtttcctccccgcagaaagcg

SEQ ID NO: 229 - DNA, 35 bp, Artificial Sequence
  Synthetic Construct
ccattgcacggcagatttccgctttctgcggggag

SEQ ID NO: 230 - DNA, 38 bp, Artificial Sequence
  Synthetic Construct
gaaatctgccgtgcaatgggtgccgaagcaatcattga

SEQ ID NO: 231 - DNA, 35 bp, Artificial Sequence
  Synthetic Construct
gccctccgcgttacgatcaatgattgcttcggcac

SEQ ID NO: 232 - DNA, 38 bp, Artificial Sequence
  Synthetic Construct
tcgtaacgcggagggctacaagttttggaaagacgagc

SEQ ID NO: 233 - DNA, 39 bp, Artificial Sequence
  Synthetic Construct
ttttgggtcctgcgtctgctcgtctttccaaaacttgta

SEQ ID NO: 234 - DNA, 36 bp, Artificial Sequence
  Synthetic Construct
agacgcaggacccaaaagaatggaaacgctttggca

SEQ ID NO: 235 - DNA, 35 bp, Artificial Sequence
  Synthetic Construct
agctcacggatgcgcttgccaaagcgtttccattc

SEQ ID NO: 236 - DNA, 32 bp, Artificial Sequence
  Synthetic Construct
agcgcatccgtgagctgactggtcgccgtggt

SEQ ID NO: 237 - DNA, 37 bp, Artificial Sequence
  Synthetic Construct
gtgttcgaaaacgatgtccagaccacggcgaccagtc

SEQ ID NO: 238 - DNA, 40 bp, Artificial Sequence
  Synthetic Construct
ctggacatcgttttcgaacaccctggtcgtgaaacgttcg

SEQ ID NO: 239 - DNA, 36 bp, Artificial Sequence
  Synthetic Construct
aacataaacgctcgcgccgaacgtttcacgaccagg

SEQ ID NO: 240 - DNA, 34 bp, Artificial Sequence
  Synthetic Construct
gcgcgagcgtttatgttactcgcaagggtggtac

SEQ ID NO: 241 - DNA, 35 bp, Artificial Sequence
  Synthetic Construct
ctcgcgcaggtggtaatagtaccacccttgcgagt

SEQ ID NO: 242 - DNA, 37 bp, Artificial Sequence
  Synthetic Construct
tattaccacctgcgcgagcacttccggttacatgcac

SEQ ID NO: 243 - DNA, 43 bp, Artificial Sequence
  Synthetic Construct
ccacagatagcggttatcgtattcgtgcatgtaaccggaagtg

SEQ ID NO: 244 - DNA, 46 bp, Artificial Sequence
  Synthetic Construct
gaatacgataaccgctatctgtggatgtccctgaagcgtattatcg

SEQ ID NO: 245 - DNA, 38 bp, Artificial Sequence
  Synthetic Construct
ttcgcgaagtgggagccgataatacgcttcagggacat

SEQ ID NO: 246 - DNA, 33 bp, Artificial Sequence
  Synthetic Construct
gctcccacttcgcgaactatcgtgaggcgtggg

SEQ ID NO: 247 - DNA, 34 bp, Artificial Sequence
  Synthetic Construct
gatcagacggttggcctcccacgcctcacgatag

SEQ ID NO: 248 - DNA, 38 bp, Artificial Sequence
  Synthetic Construct
aggccaaccgtctgatcgcgaagggtaaaattcatcca

SEQ ID NO: 249 - DNA, 42 bp, Artificial Sequence
  Synthetic Construct
gcggtaagttttagacagggttggatgaattttacccttcgc

SEQ ID NO: 250 - DNA, 38 bp, Artificial Sequence
  Synthetic Construct
accctgtctaaaacttaccgcctggaagacactggcca

SEQ ID NO: 251 - DNA, 33 bp, Artificial Sequence
  Synthetic Construct
gacgtcgtacgccgcctggccagtgtcttccag

SEQ ID NO: 252 - DNA, 32 bp, Artificial Sequence
  Synthetic Construct
ggcggcgtacgacgtccaccgcaacctgcacc

SEQ ID NO: 253 - DNA, 35 bp, Artificial Sequence
  Synthetic Construct
agtacgcccactttaccttggtgcaggttgcggtg

SEQ ID NO: 254 - DNA, 35 bp, Artificial Sequence
  Synthetic Construct
aaggtaaagtgggcgtactggcactggcgccggaa

SEQ ID NO: 255 - DNA, 32 bp, Artificial Sequence
  Synthetic Construct
ggacgcccagaccctcttccggcgccagtgcc

SEQ ID NO: 256 - DNA, 34 bp, Artificial Sequence
  Synthetic Construct
gagggtctgggcgtccgtgaccctgaaaagcgtg

SEQ ID NO: 257 - DNA, 42 bp, Artificial Sequence
  Synthetic Construct
ggttaatagcatcgatgtgttgtgcacgcttttcagggtcac

SEQ ID NO: 258 - DNA, 45 bp, Artificial Sequence
  Synthetic Construct
cacaacacatcgatgctattaaccgtttccgcaacgtctaaagag

SEQ ID NO: 259 - DNA, 41 bp, Artificial Sequence
  Synthetic Construct
cctgttcgtaggtcaaacgactctttagacgttgcggaaac

SEQ ID NO: 260 - DNA, 20 bp, Artificial Sequence
  Synthetic Construct
tcgtttgacctacgaacagg

SEQ ID NO: 261 - DNA, 19 bp, Artificial Sequence
  Synthetic Construct
atatctccttgaattcgcg

SEQ ID NO: 262 - DNA, 39 bp, Artificial Sequence
  Synthetic Construct
cgcgaattcaaggagatatataatgaaaaaggtttgcgt

SEQ ID NO: 263 - DNA, 37 bp, Artificial Sequence
  Synthetic Construct
tagtacccgcaccaataacgcaaacctttttcattat

SEQ ID NO: 264 - DNA, 33 bp, Artificial Sequence
  Synthetic Construct
tattggtgcgggtactatgggttctggtatcgc

SEQ ID NO: 265 - DNA, 30 bp, Artificial Sequence
  Synthetic Construct
ctgcgaaagcctgggcgataccagaaccca

SEQ ID NO: 266 - DNA, 30 bp, Artificial Sequence
  Synthetic Construct
ccaggctttcgcagcaaagggcttcgaagt

SEQ ID NO: 267 - DNA, 30 bp, Artificial Sequence
  Synthetic Construct
tgtcgcgcagaacgacttcgaagccctttg

SEQ ID NO: 268 - DNA, 36 bp, Artificial Sequence
  Synthetic Construct
cgttctgcgcgacattaaggatgaatttgttgatcg

SEQ ID NO: 269 - DNA, 36 bp, Artificial Sequence
  Synthetic Construct
tgaagtccaggccgcgatcaacaaattcatccttaa

SEQ ID NO: 270 - DNA, 36 bp, Artificial Sequence
  Synthetic Construct
cggcctggacttcattaacaaaaacctgtctaaact

SEQ ID NO: 271 - DNA, 43 bp, Artificial Sequence
  Synthetic Construct
tcgattttacctttctttaccagtttagacaggtttttgttaa

SEQ ID NO: 272 - DNA, 38 bp, Artificial Sequence
  Synthetic Construct
ggtaaagaaaggtaaaatcgaggaggctacgaaagtag

SEQ ID NO: 273 - DNA, 35 bp, Artificial Sequence
  Synthetic Construct
aaatacgggtcaggatttctactttcgtagcctcc

SEQ ID NO: 274 - DNA, 32 bp, Artificial Sequence
  Synthetic Construct
aaatcctgacccgtatttccggcaccgttgac

SEQ ID NO: 275 - DNA, 29 bp, Artificial Sequence
  Synthetic Construct
ggcagccatgttcaggtcaacggtgccgg

SEQ ID NO: 276 - DNA, 34 bp, Artificial Sequence
  Synthetic Construct
ctgaacatggctgccgattgtgacctggttatcg

SEQ ID NO: 277 - DNA, 35 bp, Artificial Sequence
  Synthetic Construct
cgttctactgctgcttcgataaccaggtcacaatc

SEQ ID NO: 278 - DNA, 35 bp, Artificial Sequence
  Synthetic Construct
aagcagcagtagaacgcatggacatcaagaaacag

SEQ ID NO: 279 - DNA, 36 bp, Artificial Sequence
  Synthetic Construct
tccagatcagcgaaaatctgtttcttgatgtccatg

SEQ ID NO: 280 - DNA, 33 bp, Artificial Sequence
  Synthetic Construct
attttcgctgatctggacaacatctgcaagcct

SEQ ID NO: 281 - DNA, 31 bp, Artificial Sequence
  Synthetic Construct
cgccaggatcgtttcaggcttgcagatgttg

SEQ ID NO: 282 - DNA, 33 bp, Artificial Sequence
  Synthetic Construct
gaaacgatcctggcgtctaacacttcttccctg

SEQ ID NO: 283 - DNA, 34 bp, Artificial Sequence
  Synthetic Construct
cgacttcagtgatgctcagggaagaagtgttaga

SEQ ID NO: 284 - DNA, 32 bp, Artificial Sequence
  Synthetic Construct
agcatcactgaagtcgcttccgcaaccaaacg

SEQ ID NO: 285 - DNA, 33 bp, Artificial Sequence
  Synthetic Construct
caatcaccttatccggacgtttggttgcggaag

SEQ ID NO: 286 - DNA, 36 bp, Artificial Sequence
  Synthetic Construct
tccggataaggtgattggtatgcacttctttaaccc

SEQ ID NO: 287 - DNA, 37 bp, Artificial Sequence
  Synthetic Construct
agtttcataactggtgccgggttaaagaagtgcatac

SEQ ID NO: 288 - DNA, 33 bp, Artificial Sequence
  Synthetic Construct
ggcaccagttatgaaactggtcgaagtgatccg

SEQ ID NO: 289 - DNA, 31 bp, Artificial Sequence
  Synthetic Construct
agaagtagcgatgccacggatcacttcgacc

SEQ ID NO: 290 - DNA, 32 bp, Artificial Sequence
  Synthetic Construct
tggcatcgctacttctcaggaaaccttcgacg

SEQ ID NO: 291 - DNA, 33 bp, Artificial Sequence
  Synthetic Construct
ggaagtttccttcaccgcgtcgaaggtttcctg

SEQ ID NO: 292 - DNA, 33 bp, Artificial Sequence
  Synthetic Construct
cggtgaaggaaacttccatcgccatcggtaaag

SEQ ID NO: 293 - DNA, 32 bp, Artificial Sequence
  Synthetic Construct
gcaacttctaccggatctttaccgatggcgat

SEQ ID NO: 294 - DNA, 30 bp, Artificial Sequence
  Synthetic Construct
atccggtagaagttgctgaagcaccgggtt

SEQ ID NO: 295 - DNA, 33 bp, Artificial Sequence
  Synthetic Construct
agaatacggttaactacgaaacccggtgcttca

SEQ ID NO: 296 - DNA, 37 bp, Artificial Sequence
  Synthetic Construct
tcgtagttaaccgtattctgatcccgatgattaacga

SEQ ID NO: 297 - DNA, 33 bp, Artificial Sequence
  Synthetic Construct
aggatgcctactgcttcgttaatcatcgggatc

SEQ ID NO: 298 - DNA, 30 bp, Artificial Sequence
  Synthetic Construct
agcagtaggcatcctggctgagggtatcgc

SEQ ID NO: 299 - DNA, 33 bp, Artificial Sequence
  Synthetic Construct
tcgatgtcttcaacagatgcgataccctcagcc

SEQ ID NO: 300 - DNA, 35 bp, Artificial Sequence
  Synthetic Construct
atctgttgaagacatcgacaaggcaatgaagctgg

SEQ ID NO: 301 - DNA, 35 bp, Artificial Sequence
  Synthetic Construct
ccatagggtgatttgcacccagcttcattgccttg

SEQ ID NO: 302 - DNA, 32 bp, Artificial Sequence
  Synthetic Construct
gtgcaaatcaccctatgggcccgctggagctg

SEQ ID NO: 303 - DNA, 31 bp, Artificial Sequence
  Synthetic Construct
aggccgataaaatcacccagctccagcgggc

SEQ ID NO: 304 - DNA, 32 bp, Artificial Sequence
  Synthetic Construct
ggtgattttatcggcctggacatctgtctggc

SEQ ID NO: 305 - DNA, 32 bp, Artificial Sequence
  Synthetic Construct
acagtacgtccatgatggccagacagatgtcc

SEQ ID NO: 306 - DNA, 32 bp, Artificial Sequence
  Synthetic Construct
catcatggacgtactgtactctgaaacgggcg

SEQ ID NO: 307 - DNA, 33 bp, Artificial Sequence
  Synthetic Construct
gcggacggtacttagaatcgcccgtttcagagt

SEQ ID NO: 308 - DNA, 33 bp, Artificial Sequence
  Synthetic Construct
attctaagtaccgtccgcacacgctgctgaaaa

SEQ ID NO: 309 - DNA, 30 bp, Artificial Sequence
  Synthetic Construct
cggcacgaacatactttttcagcagcgtgt

SEQ ID NO: 310 - DNA, 30 bp, Artificial Sequence
  Synthetic Construct
agtatgttcgtgccggctggctgggtcgta

SEQ ID NO: 311 - DNA, 33 bp, Artificial Sequence
  Synthetic Construct
aaaaacctttgccagatttacgacccagccagc

SEQ ID NO: 312 - DNA, 37 bp, Artificial Sequence
  Synthetic Construct
aatctggcaaaggtttttacgattactccaagtaact

SEQ ID NO: 313 - DNA, 33 bp, Artificial Sequence
  Synthetic Construct
taccgacgtcctcgagttacttggagtaatcgt

SEQ ID NO: 314 - DNA, 15 bp, Artificial Sequence
  Synthetic Construct
cgaggacgtcggtaa

SEQ ID NO: 315 - DNA, 76 bp, Artificial Sequence
  Synthetic Construct
atgaattcaagaaggagatataccatgaaagtcacgaaccagaaggaactgaagcagaaactgaacgaactgcgcg

SEQ ID NO: 316 - DNA, 70 bp, Artificial Sequence
  Synthetic Construct
atcaagcttggtaccttaaaaagatttgatataaatgtctttcagctcagagatcagcgggtaacgcggg

SEQ ID NO: 317 - DNA, 60 bp, Artificial Sequence
  Synthetic Construct
agcctcgagttaaaaagatttgatataaatgtctttcagctcagagatcagcgggtaacg

SEQ ID NO: 318 - DNA, 63 bp, Artificial Sequence
  Synthetic Construct
cggagctcttaaaaagatttgatataaatgtctttcagctcagagatcagcgggtaacgcggg

SEQ ID NO: 319 - DNA, 63 bp, Artificial Sequence; identical to SEQ ID NO: 318
  Synthetic Construct

SEQ ID NO: 320 - DNA, 59 bp, Artificial Sequence
  Synthetic Construct
gagatatacatatgacggtaaaagatattctggacgcgattcagtccaaagacgctacc

SEQ ID NO: 321 - DNA, 45 bp, Artificial Sequence
  Synthetic Construct
atggtctcccatgacggtaaaagatattctggacgcgattcagtc

SEQ ID NO: 322 - DNA, 50 bp, Artificial Sequence
  Synthetic Construct
atgaattcttacacattacgaaaacggttgatcgcgtcgatatgctgggc

SEQ ID NO: 323 - DNA, 59 bp, Artificial Sequence
  Synthetic Construct
ataccatggaactgaacaacgtgatcctggaaaaagagggtaaagtagcagttgtcacc

SEQ ID NO: 324 - DNA, 70 bp, Artificial Sequence
  Synthetic Construct
aataaagcttcccgggttaacgatttttgaagccttcgattttacgtttttcgatgaaagcggtcatggc

SEQ ID NO: 325 - DNA, 59 bp, Artificial Sequence
  Synthetic Construct
atgaattcaaggagatatataatgaaaaaggtttgcgttattggtgcgggtactatggg

SEQ ID NO: 326 - DNA, 51 bp, Artificial Sequence
  Synthetic Construct
ataaagcttggtaccgacgtcctcgagttacttggagtaatcgtaaaaacc

SEQ ID NO: 327 - DNA, 60 bp, Artificial Sequence
  Synthetic Construct
cggagctcaaggagatatacatatgactgatgttgtgattgtaagcgctgcacgtactgc

SEQ ID NO: 328 - DNA, 38 bp, Artificial Sequence
  Synthetic Construct
agatctagattagcccatgtgcaggccaccgttcaggg

SEQ ID NO: 329 - DNA, 44 bp, Artificial Sequence
  Synthetic Construct
atcgcccttccggccgactgcacggtgcaccaatgcttctggcg

SEQ ID NO: 330 - DNA, 57 bp, Artificial Sequence
  Synthetic Construct
atcgcccttccggcaagagtttgtagaaacgcaaaaaggccatccgtcaggatggcc

SEQ ID NO: 331 - DNA, 60 bp, Artificial Sequence
  Synthetic Construct
tacatatgagatctctcgagctcgagggctgttttggcggatgagagaagattttcagcc

SEQ ID NO: 332 - DNA, 40 bp, Artificial Sequence
  Synthetic Construct
ttgaagcatttatcagggttattgtctcatgagcggatac

SEQ ID NO: 333 - DNA, 35 bp, Artificial Sequence
  Synthetic Construct
gagatatacatatgatcgtcaagccaatggtgcgc

SEQ ID NO: 334 - DNA, 58 bp, Artificial Sequence
  Synthetic Construct
ctccttgaattcttaaatacgatcgaaacgttcaacttctgcctcgtagttaataccc

SEQ ID NO: 335 - DNA, 2391 bp, Escherichia coli
atggcaattaacaatacaggctcgcgacgattactcgtcacgctaacagccctttttgcagcgctttgcgggctgtatctactcattggcggaggctggctggtcgcgattggcggctcctggtactaccctatcgctggccttgtgatgctcggcgtcgcctggatgctgtggcgcagtaaacgcgccgcgctttggctatacgcagccctgctgctcggcaccatgatttggggcgtctgggaagttggtttcgacttctgggcgctgactccgcgcagcgacattctggtcttcttcggcatctggctgatcctgccgtttgtctggcgtcgcctggtcattcctgccagcggcgcagttgccgcactggtggtcgcactgctgattagcggtggtatcctgacctgggccggatttaacgatccgcaggagatcaacggcaccttaagcgccgatgccacacctgctgaagctatctcccccgtagccgatcaggactggcctgcctatggtcgtaatcaggaaggtcaacgcttttcgccgctgaaacaaattaacgccgataacgtccataatctgaaagaagcctgggtgttccgtactggcgatgtgaagcagccgaacgatccgggtgaaatcaccaatgaagtgacgccgattaaagtgggcgacaccctttacctgtgtaccgctcaccagcgcctgtttgcgcttgatgccgccagcggcaaagagaaatggcattacgatcctgagctgaaaaccaacgagtctttccagcacgtaacctgccgtggtgtctcttatcatgaagccaaagcagaaaccgcttcgccggaagtgatggcggattgcccgcgtcgtatcattcttccggtcaatgatggtcgactgattgcgattaacgctgaaaacggcaaactgtgcgaaaccttcgccaataaaggcgtgctcaatctgcaaagcaatatgccagacaccaaaccgggtctgtatgaaccgacttcgccaccgattatcaccgataaaaccatcgtgatggccggttcagttaccgataacttctcaacccgcgaaacgtctggcgtgatccgtggttttgatgtcaacaccggggagctgctgtgggcttttgatcccggcgcgaaagatccgaacgcaatcccgtctgacgaacacacctttacctttaactcgccaaactcctgggcaccagcggcctatgacgcgaagctggatctggtctatctgccgatgggcgtgaccacgccggatatctggggcggtaaccgcacaccggaacaggaacgttatgccagctcgattctggcgctgaatgccactaccgggaaactggcgtggagctaccagaccgttcaccacgacctgtgggacatggatcttccggcacagccgacgctggcggacatcaccgttaatggtcagaaagtgccagttatttacgctccggcgaaaaccggcaacatttttgtgctcgatcgtcgtaatggcgaactggtggttccggcaccggaaaaaccggttccccaaggtgcagcgaaaggcgattacgtaaccccaactcaaccgttttctgaactgagcttccgtccgacgaaagatttgagcggtgcggatatgtggggagccaccatgtttgaccaactggtgtgccgcgtgatgttccaccagatgcgctatgaaggcattttcaccccgccatctgaacagggtacgctggtcttcccgggtaacctggggatgttcgaatggggcgggatttccgttgatccaaatcgtgaagtggcgattgccaacccaatggcactgccgtttgtttcgaaactgatcccgcgtggtcctggcaacccgatggagcagccgaaagatgccaaaggcacgggtacggaatccggcattcagccacagtacggtgtaccgtatggtgtcacgctcaacccgttcctctcaccatttggtctgccatgtaaacagccagcatggggttatatctcggcgctggatctgaaaactaatgaagtggtgtggaagaaacgtattggtacgccgcaggacagtatgccgttcccgatgccggttccggtgccgttcaatatgggtatgccgatgctgggcgggccaatctccacggcgggtaacgtgctgtttatcgccgctacggcagataactacctgcgcgcttacaacatgagcaacggtgaaaaactgtggcagggtcgtttaccagcgggtggtcaggctacgccaatgacctatgaagtgaatggtaagcagtatgtggtgatctccgcaggcggtcacggttcatttggtacgaagatgggcgactatattgtggcttatgcgctgccggatgatgtgaagtaa

SEQ ID NO: 336 - protein, 796 aa, Escherichia coli
MAINNTGSRRLLVTLTALFAALCGLYLLIGGGWLVAIGGSWYYPIAGLVMLGVAWMLWRSKRAALWLYAALLLGTMIWGVWEVGFDFWALTPRSDILVFFGIWLILPFVWRRLVIPASGAVAALVVALLISGGILTWAGFNDPQEINGTLSADATPAEAISPVADQDWPAYGRNQEGQRFSPLKQINADNVHNLKEAWVFRTGDVKQPNDPGEITNEVTPIKVGDTLYLCTAHQRLFALDAASGKEKWHYDPELKTNESFQHVTCRGVSYHEAKAETASPEVMADCPRRIILPVNDGRLIAINAENGKLCETFANKGVLNLQSNMPDTKPGLYEPTSPPIITDKTIVMAGSVTDNFSTRETSGVIRGFDVNTGELLWAFDPGAKDPNAIPSDEHTFTFNSPNSWAPAAYDAKLDLVYLPMGVTTPDIWGGNRTPEQERYASSILALNATTGKLAWSYQTVHHDLWDMDLPAQPTLADITVNGQKVPVIYAPAKTGNIFVLDRRNGELVVPAPEKPVPQGAAKGDYVTPTQPFSELSFRPTKDLSGADMWGATMFDQLVCRVMFHQMRYEGIFTPPSEQGTLVFPGNLGMFEWGGISVDPNREVAIANPMALPFVSKLIPRGPGNPMEQPKDAKGTGTESGIQPQYGVPYGVTLNPFLSPFGLPCKQPAWGYISALDLKTNEVVWKKRIGTPQDSMPFPMPVPVPFNMGMPMLGGPISTAGNVLFIAATADNYLRAYNMSNGEKLWQGRLPAGGQATPMTYEVNGKQYVVISAGGHGSFGTKMGDYIVAYALPDDVK

SEQ ID NO: 337 - DNA, 975 bp, Escherichia coli
atgaagccgtccgttatcctctacaaagccttacctgatgatttactgcaacgcctgcaagagcatttcaccgttcaccaggtggcaaacctcagcccacaaaccgtcgaacaaaatgcagcaatttttgccgaagctgaaggtttactgggttcaaacgagaatgtaaatgccgcattgctggaaaaaatgccgaaactgcgtgccacatcaacgatctccgtcggctatgacaattttgatgtcgatgcgcttaccgcccgaaaaattctgctgatgcacacgccaaccgtattaacagaaaccgtcgccgatacgctgatggcgctggtgttgtctaccgctcgtcgggttgtggaggtagcagaacgggtaaaagcaggcgaatggaccgcgagcataggcccggactggtacggcactgacgttcaccataaaacactgggcattgtcgggatgggacggatcggcatggcgctggcacaacgtgcgcactttggcttcaacatgcccatcctctataacgcgcgccgccaccataaagaagcagaagaacgcttcaacgcccgctactgcgatttggatactctgttacaagagtcagatttcgtttgcctgatcctgccgttaactgatgagacgcatcatctgtttggcgcagaacaattcgccaaaatgaaatcctccgccattttcattaatgccggacgtggcccggtggttgacgaaaatgcactgatcgcagcattgcagaaaggcgaaattcacgctgccgggctggatgtcttcgaacaagagccactgtccgtagattcgccgttgctctcaatggccaacgtcgtcgcagtaccgcatattggatctgccacccatgagacgcgttatggcatggccgcctgtgccgtggataatttgattgatgcgttacaaggaaaggttgagaagaactgtgtgaatccgcacgtcgcggactaa

SEQ ID NO: 338 - protein, 324 aa, Escherichia coli
MKPSVILYKALPDDLLQRLQEHFTVHQVANLSPQTVEQNAAIFAEAEGLLGSNENVNAALLEKMPKLRATSTISVGYDNFDVDALTARKILLMHTPTVLTETVADTLMALVLSTARRVVEVAERVKAGEWTASIGPDWYGTDVHHKTLGIVGMGRIGMALAQRAHFGFNMPILYNARRHHKEAEERFNARYCDLDTLLQESDFVCLILPLTDETHHLFGAEQFAKMKSSAIFINAGRGPVVDENALIAALQKGEIHAAGLDVFEQEPLSVDSPLLSMANVVAVPHIGSATHETRYGMAACAVDNLIDALQGKVEKNCVNPHVAD

SEQ ID NO: 339 - DNA, 528 bp, Escherichia coli
ttgagcacgactaaccatgatcaccacatttacgtcttgatgggcgtatcgggcagcggcaaatctgcggtcgccagtgaagtggcgcatcaacttcatgccgcgtttcttgatggcgatttcctccatccacggcgcaatatcgaaaaaatggcgtctggcgaaccactgaatgacgacgatcgcaaaccgtggttgcaggcgctgaacgacgccgcgtttgctatgcagcgcactaataaagtgtcgctgatcgtctgttctgcattgaaaaaacactatcgcgacttgctgcgtgaaggtaatccgaatctctctttcatctatttgaaaggcgattttgatgtgattgaaagccgcctgaaagcgcgcaaaggccatttctttaaaacccaaatgttggtgacgcagtttgaaacgctgcaggagccgggtgcggacgaaaccgatgtactggtggtggatatcgatcaaccgctggaaggtgttgtggcaagcaccattgaggttattaaaaaaggcaaataa

SEQ ID NO: 340 - protein, 175 aa, Escherichia coli
MSTTNHDHHIYVLMGVSGSGKSAVASEVAHQLHAAFLDGDFLHPRRNIEKMASGEPLNDDDRKPWLQALNDAAFAMQRTNKVSLIVCSALKKHYRDLLREGNPNLSFIYLKGDFDVIESRLKARKGHFFKTQMLVTQFETLQEPGADETDVLVVDIDQPLEGVVASTIEVIKKGK

SEQ ID NO: 341 - DNA, 564 bp, Escherichia coli
atggcgggtgaaagctttattttgatgggcgtttcagggagtggtaaaacattaattggtagcaaggttgccgcgttattatctgctaaatttattgatggtgacgatcttcatccagccaaaaatatagataaaatgtcgcagggtattccattatctgatgaagatcgacttccctggctggaacgcttaaatgatgcttcatacagtctttataaaaagaatgaaacaggatttattgtctgttcatcattaaaaaaacagtatcgtgatattttacgcaagggtagcccccatgttcatttcctctggttagatggcgactatgaaactattctcgcgcgaatgcagcgtcgggctgggcattttatgccggtagcgttactaaaaagtcagtttgaggcactggagcgtccacaagcagatgaacaggatattgtgcgcattgatatcaaccatgatattgccaatgtcaccgaacaatgtcggcaggctgtgctggcgatacgacaaaacagaatatgtgcgaaagagggcagcgcctcagatcagcgctgcgaatga

SEQ ID NO: 342 - protein, 187 aa, Escherichia coli
MAGESFILMGVSGSGKTLIGSKVAALLSAKFIDGDDLHPAKNIDKMSQGIPLSDEDRLPWLERLNDASYSLYKKNETGFIVCSSLKKQYRDILRKGSPHVHFLWLDGDYETILARMQRRAGHFMPVALLKSQFEALERPQADEQDIVRIDINHDIANVTEQCRQAVLAIRQNRICAKEGSASDQRCE

SEQ ID NO: 343 - DNA, 930 bp, Escherichia coli
atgtccaaaaagattgccgtgattggcgaatgcatgattgagctttccgagaaaggcgcggacgttaagcgcggtttcggcggcgataccctgaacacttccgtctatatcgcccgtcaggtcgatcctgcggcattaaccgttcattacgtaacggcgctgggaacggacagttttagccagcagatgctggacgcctggcacggcgagaacgttgatacttccctgacccaacggatggaaaaccgtctgccgggcctttactacattgaaaccgacagcaccggcgagcgtacgttctactactggcggaacgaagccgccgccaaattctggctggagagtgagcagtctgcggcgatttgcgaagagctggcgaatttcgattatctctacctgagcgggattagcctggcgatcttaagcccgaccagccgcgaaaagctgctttccctgctgcgcgaatgccgcgccaacggcggaaaagtgattttcgacaataactatcgtccgcgcctgtgggccagcaaagaagagacacagcaggtgtaccaacaaatgctggaatgcacggatatcgccttcctgacgctggacgacgaagacgcgctgtggggtcaacagccggtggaagacgtcattgcgcgcacccataacgcgggcgtgaaagaagtggtggtgaaacgcggggcggattcttgcctggtgtccattgctggcgaagggttagtggatgttccggcggtgaaactgccgaaagaaaaagtgatcgataccaccgcagctggcgactctttcagtgccggttatctggcggtacgtctgacaggcggcagcgcggaagacgcggcgaaacgtgggcacctgaccgcaagtaccgttattcagtatcgcggcgcgattatcccgcgtgaggcgatgccagcgtaa

SEQ ID NO: 344 - protein, 309 aa, Escherichia coli
MSKKIAVIGECMIELSEKGADVKRGFGGDTLNTSVYIARQVDPAALTVHYVTALGTDSFSQQMLDAWHGENVDTSLTQRMENRLPGLYYIETDSTGERTFYYWRNEAAAKFWLESEQSAAICEELANFDYLYLSGISLAILSPTSREKLLSLLRECRANGGKVIFDNNYRPRLWASKEETQQVYQQMLECTDIAFLTLDDEDALWGQQPVEDVIARTHNAGVKEVVVKRGADSCLVSIAGEGLVDVPAVKLPKEKVIDTTAAGDSFSAGYLAVRLTGGSAEDAAKRGHLTASTVIQYRGAIIPREAMPA

SEQ ID NO: 345 - DNA, 1650 bp, Escherichia coli
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 346 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 347 - DNA, 1476 bp, Escherichia coli
atggcggtaacgcaaacagcccaggcctgtgacctggtcattttcggcgcgaaaggcgaccttgcgcgtcgtaaattgctgccttccctgtatcaactggaaaaagccggtcagctcaacccggacacccggattatcggcgtagggcgtgctgactgggataaagcggcatataccaaagttgtccgcgaggcgctcgaaactttcatgaaagaaaccattgatgaaggtttatgggacaccctgagtgcacgtctggatttttgtaatctcgatgtcaatgacactgctgcattcagccgtctcggcgcgatgctggatcaaaaaaatcgtatcaccattaactactttgccatgccgcccagcacttttggcgcaatttgcaaagggcttggcgaggcaaaactgaatgctaaaccggcacgcgtagtcatggagaaaccgctggggacgtcgctggcgacctcgcaggaaatcaatgatcaggttggcgaatacttcgaggagtgccaggtttaccgtatcgaccactatcttggtaaagaaacggtgctgaacctgttggcgctgcgttttgctaactccctgtttgtgaataactgggacaatcgcaccattgatcatgttgagattaccgtggcagaagaagtggggatcgaagggcgctggggctattttgataaagccggtcagatgcgcgacatgatccagaaccacctgctgcaaattctttgcatgattgcgatgtctccgccgtctgacctgagcgcagacagcatccgcgatgaaaaagtgaaagtactgaagtctctgcgccgcatcgaccgctccaacgtacgcgaaaaaaccgtacgcgggcaatatactgcgggcttcgcccagggcaaaaaagtgccgggatatctggaagaagagggcgcgaacaagagcagcaatacagaaactttcgtggcgatccgcgtcgacattgataactggcgctgggccggtgtgccattctacctgcgtactggtaaacgtctgccgaccaaatgttctgaagtcgtggtctatttcaaaacacctgaactgaatctgtttaaagaatcgtggcaggatctgccgcagaataaactgactatccgtctgcaacctgatgaaggcgtggatatccaggtactgaataaagttcctggccttgaccacaaacataacctgcaaatcaccaagctggatctgagctattcagaaacctttaatcagacgcatctggcggatgcctatgaacgtttgctgctggaaaccatgcgtggtattcaggcactgtttgtacgtcgcgacgaagtggaagaagcctggaaatgggtagactccattactgaggcgtgggcgatggacaatgatgcgccgaaaccgtatcaggccggaacctggggacccgttgcctcggtggcgatgattacccgtgatggtcgttcctggaatgagtttgagtaa

SEQ ID NO: 348 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 349 - DNA, 996 bp, Escherichia coli
atgaagcaaacagtttatatcgccagccctgagagccagcaaattcacgtctggaatctgaatcatgaaggcgcactgacgctgacacaggttgtcgatgtgccggggcaggtgcagccgatggtggtcagcccggacaaacgttatctctatgttggtgttcgccctgagtttcgcgtcctggcgtatcgtatcgccccggacgatggcgcactgacctttgccgcagagtctgcgctgccgggtagtccgacgcatatttccaccgatcaccaggggcagtttgtctttgtaggttcttacaatgcgggtaacgtgagcgtaacgcgtctggaagatggcctgccagtgggcgtcgtcgatgtggtcgaggggctggacggttgccattccgccaatatctcaccggacaaccgtacgctgtgggttccggcattaaagcaggatcgcatttgcctgtttacggtcagcgatgatggtcatctcgtggcgcaggaccctgcggaagtgaccaccgttgaaggggccggcccgcgtcatatggtattccatccaaacgaacaatatgcgtattgcgtcaatgagttaaacagctcagtggatgtctgggaactgaaagatccgcacggtaatatcgaatgtgtccagacgctggatatgatgccggaaaacttctccgacacccgttgggcggctgatattcatatcaccccggatggtcgccatttatacgcctgcgaccgtaccgccagcctgattaccgttttcagcgtttcggaagatggcagcgtgttgagtaaagaaggcttccagccaacggaaacccagccgcgcggcttcaatgttgatcacagcggcaagtatctgattgccgccgggcaaaaatctcaccacatctcggtatacgaaattgttggcgagcaggggctactgcatgaaaaaggccgctatgcggtcgggcagggaccaatgtgggtggtggttaacgcacactaa

SEQ ID NO: 350 - protein, 331 aa, Escherichia coli
MKQTVYIASPESQQIHVWNLNHEGALTLTQVVDVPGQVQPMVVSPDKRYLYVGVRPEFRVLAYRIAPDDGALTFAAESALPGSPTHISTDHQGQFVFVGSYNAGNVSVTRLEDGLPVGVVDVVEGLDGCHSANISPDNRTLWVPALKQDRICLFTVSDDGHLVAQDPAEVTTVEGAGPRHMVFHPNEQYAYCVNELNSSVDVWELKDPHGNIECVQTLDMMPENFSDTRWAADIHITPDGRHLYACDRTASLITVFSVSEDGSVLSKEGFQPTETQPRGFNVDHSGKYLIAAGQKSHHISVYEIVGEQGLLHEKGRYAVGQGPMWVVVNAH

SEQ ID NO: 351 - DNA, 1812 bp, Escherichia coli
atgaatccacaattgttacgcgtaacaaatcgaatcattgaacgttcgcgcgagactcgctctgcttatctcgcccggatagaacaagcgaaaacttcgaccgttcatcgttcgcagttggcatgcggtaacctggcacacggtttcgctgcctgccagccagaagacaaagcctctttgaaaagcatgttgcgtaacaatatcgccatcatcacctcctataacgacatgctctccgcgcaccagccttatgaacactatccagaaatcattcgtaaagccctgcatgaagcgaatgcggttggtcaggttgcgggcggtgttccggcgatgtgtgatggtgtcacccaggggcaggatggaatggaattgtcgctgctaagccgcgaagtgatagcgatgtctgcggcggtggggctgtcccataacatgtttgatggtgctctgttcctcggtgtgtgcgacaagattgtcccgggtctgacgatggcagccctgtcgtttggtcatttgcctgcggtgtttgtgccgtctggaccgatggcaagcggtttgccaaataaagaaaaagtgcgtattcgccagctttatgccgaaggtaaagtggaccgcatggccttactggagtcagaagccgcgtcttaccatgcgccgggaacatgtactttctacggtactgccaacaccaaccagatggtggtggagtttatggggatgcagttgccaggctcttcttttgttcatccggattctccgctgcgcgatgctttgaccgccgcagctgcgcgtcaggttacacgcatgaccggtaatggtaatgaatggatgccgatcggtaagatgatcgatgagaaagtggtggtgaacggtatcgttgcactgctggcgaccggtggttccactaaccacaccatgcacctggtggcgatggcgcgcgcggccggtattcagattaactgggatgacttctctgacctttctgatgttgtaccgctgatggcacgtctctacccgaacggtccggccgatattaaccacttccaggcggcaggtggcgtaccggttctggtgcgtgaactgctcaaagcaggcctgctgcatgaagatgtcaatacggtggcaggttttggtctgtctcgttatacccttgaaccatggctgaataatggtgaactggactggcgggaaggggcggaaaaatcactcgacagcaatgtgatcgcttccttcgaacaacctttctctcatcatggtgggacaaaagtgttaagcggtaacctgggccgtgcggttatgaaaacctctgccgtgccggttgagaaccaggtgattgaagcgccagcggttgtttttgaaagccagcatgacgttatgccggcctttgaagcgggtttgctggaccgcgattgtgtcgttgttgtccgtcatcaggggccaaaagcgaacggaatgccagaattacataaactcatgccgccacttggtgtattattggaccggtgtttcaaaattgcgttagttaccgatggacgactctccggcgcttcaggtaaagtgccgtcagctatccacgtaacaccagaagcctacgatggcgggctgctggcaaaagtgcgcgacggggacatcattcgtgtgaatggacagacaggcgaactgacgctgctggtagacgaagcggaactggctgctcgcgaaccgcacattcctgacctgagcgcgtcacgcgtgggaacaggacgtgaattattcagcgccttgcgtgaaaaactgtccggtgccgaacagggcgcaacctgtatcactttttaa

SEQ ID NO: 352 - protein, 603 aa, Escherichia coli
MNPQLLRVTNRIIERSRETRSAYLARIEQAKTSTVHRSQLACGNLAHGFAACQPEDKASLKSMLRNNIAIITSYNDMLSAHQPYEHYPEIIRKALHEANAVGQVAGGVPAMCDGVTQGQDGMELSLLSREVIAMSAAVGLSHNMFDGALFLGVCDKIVPGLTMAALSFGHLPAVFVPSGPMASGLPNKEKVRIRQLYAEGKVDRMALLESEAASYHAPGTCTFYGTANTNQMVVEFMGMQLPGSSFVHPDSPLRDALTAAAARQVTRMTGNGNEWMPIGKMIDEKVVVNGIVALLATGGSTNHTMHLVAMARAAGIQINWDDFSDLSDVVPLMARLYPNGPADINHFQAAGGVPVLVRELLKAGLLHEDVNTVAGFGLSRYTLEPWLNNGELDWREGAEKSLDSNVIASFEQPFSHHGGTKVLSGNLGRAVMKTSAVPVENQVIEAPAVVFESQHDVMPAFEAGLLDRDCVVVVRHQGPKANGMPELHKLMPPLGVLLDRCFKIALVTDGRLSGASGKVPSAIHVTPEAYDGGLLAKVRDGDIIRVNGQTGELTLLVDEAELAAREPHIPDLSASRVGTGRELFSALREKLSGAEQGATCITF

SEQ ID NO: 353 - DNA, 642 bp, Escherichia coli
atgaaaaactggaaaacaagtgcagaatcaatcctgaccaccggcccggttgtaccggttatcgtggtaaaaaaactggaacacgcggtgccgatggcaaaagcgttggttgctggtggggtgcgcgttctggaagtgactctgcgtaccgagtgtgcagttgacgctatccgtgctatcgccaaagaagtgcctgaagcgattgtgggtgccggtacggtgctgaatccacagcagctggcagaagtcactgaagcgggtgcacagttcgcaattagcccgggtctgaccgagccgctgctgaaagctgctaccgaagggactattcctctgattccggggatcagcactgtttccgaactgatgctgggtatggactacggtttgaaagagttcaaattcttcccggctgaagctaacggcggcgtgaaagccctgcaggcgatcgcgggtccgttctcccaggtccgtttctgcccgacgggtggtatttctccggctaactaccgtgactacctggcgctgaaaagcgtgctgtgcatcggtggttcctggctggttccggcagatgcgctggaagcgggcgattacgaccgcattactaagctggcgcgtgaagctgtagaaggcgctaagctgtaa

SEQ ID NO: 354 - protein, 213 aa, Escherichia coli
MKNWKTSAESILTTGPVVPVIVVKKLEHAVPMAKALVAGGVRVLEVTLRTECAVDAIRAIAKEVPEAIVGAGTVLNPQQLAEVTEAGAQFAISPGLTEPLLKAATEGTIPLIPGISTVSELMLGMDYGLKEFKFFPAEANGGVKALQAIAGPFSQVRFCPTGGISPANYRDYLALKSVLCIGGSWLVPADALEAGDYDRITKLAREAVEGAKL

SEQ ID NO: 355 - DNA, 1407 bp, Escherichia coli
atgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 356 - protein, 468 aa, Escherichia coli
MSKQQIGVVGMAVMGRNLALNIESRGYTVSIFNRSREKTEEVIAENPGKKLVPYYTVKEFVESLETPRRILLMVKAGAGTDAAIDSLKPYLDKGDIIIDGGNTFFQDTIRRNRELSAEGFNFIGTGVSGGEEGALKGPSIMPGGQKEAYELVAPILTKIAAVAEDGEPCVTYIGADGAGHYVKMVHNGIEYGDMQLIAEAYSLLKGGLNLTNEELAQTFTEWNNGELSSYLIDITKDIFTKKDEDGNYLVDVILDEAANKGTGKWTSQSALDLGEPLSLITESVFARYISSLKDQRVAASKVLSGPQAQPAGDKAEFIEKVRRALYLGKIVSYAQGFSQLRAASEEYNWDLNYGEIAKIFRAGCIIRAQFLQKITDAYAENPQIANLLLAPYFKQIADDYQQALRDVVAYAVQNGIPVPTFSAAVAYYDSYRAAVLPANLIQAQRDYFGAHTYKRIDKEGVFHTEWLD

SEQ ID NO: 357 - DNA, 2004 bp, Escherichia coli
atgtcccgaaaagaccttgccaatgcgattcgcgcactcagtatggatgcggtacaaaaagccaactctggtcatcccggcgcgccgatgggcatggctgatattgccgaagtgctgtggaacgattttcttaaacataaccctaccgacccaacctggtatgatcgcgaccgctttattctttccaacggtcacgcgtcgatgctgctctacagtttgctacatctgaccggttacgacctgccgctggaagaactgaagaacttccgtcagttgcattcgaaaaccccaggccacccggagattggctatacgccaggcgttgaaaccaccaccggcccgcttggacaaggtttggcgaacgccgtcgggctggcgatagcagagcgtacactggcggcgcagtttaaccagccagaccatgagatcgtcgatcacttcacctatgtgtttatgggcgacggctgcctgatggaaggtatttcccacgaagtctgttcgctggcaggcacgctgggactgggcaagctgattggtttttacgatcacaacggtatttccatcgacggtgaaacagaaggctggtttaccgacgatacggcaaaacgttttgaagcctatcactggcatgtgatccatgaaatcgacggtcacgatccgcaggcggtgaaggaagcgatccttgaagcgcaaagcgtgaaagataagccgtcgctgattatctgccgtacggtgattggctttggttcgccgaataaagcaggtaaggaagaggcgcacggcgcaccactgggggaagaagaagtggcgctggcacggcaaaaactgggctggcaccatccgccatttgagatccctaaagagatttatcacgcctgggatgcccgtgaaaaaggcgaaaaagcgcagcagagctggaatgagaagtttgccgcctataaaaaggctcatccgcaactggcagaagagtttacccgacggatgagcggtggtttaccgaaggactgggagaaaacgactcagaaatatatcaatgagttacaggcaaatccggcgaaaatcgctacccgtaaggcttcgcaaaatacgcttaacgcttacgggccgatgctgcctgagttgctcggcggttcggcggatctggctcccagcaacctgaccatctggaaaggttctgtttcgctgaaggaagatccagcgggcaactacattcactacggggtgcgtgaatttggcatgaccgctatcgccaacggcatcgcgcaccacggcggctttgtgccgtataccgcgacgttcctgatgtttgttgaatacgcccgtaacgccgcgcggatggcggcactgatgaaagcgcggcagattatggtttatacccacgactcaattggcctgggcgaagatggtccgacgcaccaggctgttgagcaactggccagcctgcgcttaacgccaaatttcagcacctggcgaccgtgcgatcaggtggaagcggcggtgggctggaagctggcggttgagcgccacaacggaccgacggcactgatcctctcaaggcagaatctggcccaggtggaacgtacgccggatcaggttaaagagattgctcgtggcggttatgtgctgaaagacagcggcggtaagccagatattattctgattgccaccggttcagagatggaaattaccctgcaagcggcagagaaattagcaggagaaggtcgcaatgtacgcgtagtttccctgccctcgaccgatattttcgacgcccaggatgaggaatatcgggagtcggtgttgccttctaacgttgcggctcgcgtggcggtggaagcaggtattgccgattactggtacaagtatgttggtctgaaaggggcaattgtcgggatgacgggttacggggaatctgctccggcggataagctgttcccgttctttggctttaccgccgagaatattgtggcaaaagcgcataaggtgctgggagtgaaaggtgcctga

SEQ ID NO: 358 - protein, 667 aa, Escherichia coli
MSRKDLANAIRALSMDAVQKANSGHPGAPMGMADIAEVLWNDFLKHNPTDPTWYDRDRFILSNGHASMLLYSLLHLTGYDLPLEELKNFRQLHSKTPGHPEIGYTPGVETTTGPLGQGLANAVGLAIAERTLAAQFNQPDHEIVDHFTYVFMGDGCLMEGISHEVCSLAGTLGLGKLIGFYDHNGISIDGETEGWFTDDTAKRFEAYHWHVIHEIDGHDPQAVKEAILEAQSVKDKPSLIICRTVIGFGSPNKAGKEEAHGAPLGEEEVALARQKLGWHHPPFEIPKEIYHAWDAREKGEKAQQSWNEKFAAYKKAHPQLAEEFTRRMSGGLPKDWEKTTQKYINELQANPAKIATRKASQNTLNAYGPMLPELLGGSADLAPSNLTIWKGSVSLKEDPAGNYIHYGVREFGMTAIANGIAHHGGFVPYTATFLMFVEYARNAARMAALMKARQIMVYTHDSIGLGEDGPTHQAVEQLASLRLTPNFSTWRPCDQVEAAVGWKLAVERHNGPTALILSRQNLAQVERTPDQVKEIARGGYVLKDSGGKPDIILIATGSEMEITLQAAEKLAGEGRNVRVVSLPSTDIFDAQDEEYRESVLPSNVAARVAVEAGIADYWYKYVGLKGAIVGMTGYGESAPADKLFPFFGFTAENIVAKAHKVLGVKGA

SEQ ID NO: 359 - DNA, 1992 bp, Escherichia coli
atgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacggcccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaa

SEQ ID NO: 360 - protein, 663 aa, Escherichia coli
MSSRKELANAIRALSMDAVQKAKSGHPGAPMGMADIAEVLWRDFLKHNPQNPSWADRDRFVLSNGHGSMLIYSLLHLTGYDLPMEELKNFRQLHSKTPGHPEVGYTAGVETTTGPLGQGIANAVGMAIAEKTLAAQFNRPGHDIVDHYTYAFMGDGCMMEGISHEVCSLAGTLKLGKLIAFYDDNGISIDGHVEGWFTDDTAMRFEAYGWHVIRDIDGHDAASIKRAVEEARAVTDKPSLLMCKTIIGFGSPNKAGTHDSHGAPLGDAEIALTREQLGWKYAPFEIPSEIYAQWDAKEAGQAKESAWNEKFAAYAKAYPQEAAEFTRRMKGEMPSDFDAKAKEFIAKLQANPAKIASRKASQNAIEAFGPLLPEFLGGSADLAPSNLTLWSGSKAINEDAAGNYIHYGVREFGMTAIANGISLHGGFLPYTSTFLMFVEYARNAVRMAALMKQRQVMVYTHDSIGLGEDGPTHQPVEQVASLRVTPNMSTWRPCDQVESAVAWKYGVERQDGPTALILSRQNLAQQERTEEQLANIARGGYVLKDCAGQPELIFIATGSEVELAVAAYEKLTAEGVKARVVSMPSTDAFDKQDAAYRESVLPKAVTARVAVEAGIADYWYKYVGLNGAIVGMTTFGESAPAELLFEEFGFTVDNVVAKAKELL

SEQ ID NO: 361 - DNA, 678 bp, Escherichia coli
atgaaacagtatttgattgccccctcaattctgtcggctgattttgcccgcctgggtgaagataccgcaaaagccctggcagctggcgctgatgtcgtgcattttgacgtcatggataaccactatgttcccaatctgacgattgggccaatggtgctgaaatccttgcgtaactatggcattaccgcccctatcgacgtacacctgatggtgaaacccgtcgatcgcattgtgcctgatttcgctgccgctggtgccagcatcattacctttcatccagaagcctccgagcatgttgaccgcacgctgcaactgattaaagaaaatggctgtaaagcgggtctggtatttaacccggcgacacctctgagctatctggattacgtgatggataagctggatgtgatcctgctgatgtccgtcaaccctggtttcggcggtcagtctttcattcctcaaacactggataaactgcgcgaagtacgtcgccgtatcgacgagtctggctttgacattcgactagaagtggacggtggcgtgaaggtgaacaacattggcgaaatcgctgcggcgggcgcggatatgttcgtcgccggttcggcaatcttcgaccagccagactacaaaaaagtcattgatgaaatgcgcagtgaactggcaaaggtaagtcatgaataa

SEQ ID NO: 362 - protein, 225 aa, Escherichia coli
MKQYLIAPSILSADFARLGEDTAKALAAGADVVHFDVMDNHYVPNLTIGPMVLKSLRNYGITAPIDVHLMVKPVDRIVPDFAAAGASIITFHPEASEHVDRTLQLIKENGCKAGLVFNPATPLSYLDYVMDKLDVILLMSVNPGFGGQSFIPQTLDKLREVRRRIDESGFDIRLEVDGGVKVNNIGEIAAAGADMFVAGSAIFDQPDYKKVIDEMRSELAKVSHE

SEQ ID NO: 363 - DNA, 1011 bp, Escherichia coli
atgagacgagaacttgccatcgaattttcccgcgtcaccgaatcagcggcgctggctggctacaaatggttaggacgcggcgataaaaacaccgcggacggcgcggcggtaaacgccatgcgtattatgctcaaccaggtcaacattgacggcaccatcgtcattggtgaaggtgaaatcgacgaagcaccgatgctctacattggtgaaaaagtcggtactggtcgcggcgacgcggtagatattgctgttgatccgattgaaggcacgcgcatgacggcgatgggccaggctaacgcgctggcggtgctggcagtaggcgataaaggctgcttcctcaatgcgccggatatgtatatggagaagctgattgtcgggccgggagccaaaggcaccattgatctgaacctgccgctggcggataacctgcgcaatgtagcggcggcgctcggcaaaccgttgagcgaactgacggtaacgattctggctaaaccacgccacgatgccgttatcgctgaaatgcagcaactcggcgtacgcgtatttgctattccggacggcgacgttgcggcctcaattctcacctgtatgccagacagcgaagttgacgtgctgtacggtattggtggcgcgccggaaggcgtagtttctgcggcggtgatccgcgcattagatggcgacatgaacggtcgtctgctggcgcgtcatgacgtcaaaggcgacaacgaagagaatcgtcgcattggcgagcaggagctggcacgctgcaaagcgatgggcatcgaagccggtaaagtattgcgcctgggcgatatggcgcgcagcgataacgtcatcttctctgccaccggtattaccaaaggcgatctgctggaaggcattagccgcaaaggcaatatcgcgactaccgaaacgctgctgatccgcggcaagtcacgcaccattcgccgcattcagtccatccactatctggatcgcaaagacccggaaatgcaggtgcacatcctctga

SEQ ID NO: 364 - protein, 336 aa, Escherichia coli
MRRELAIEFSRVTESAALAGYKWLGRGDKNTADGAAVNAMRIMLNQVNIDGTIVIGEGEIDEAPMLYIGEKVGTGRGDAVDIAVDPIEGTRMTAMGQANALAVLAVGDKGCFLNAPDMYMEKLIVGPGAKGTIDLNLPLADNLRNVAAALGKPLSELTVTILAKPRHDAVIAEMQQLGVRVFAIPDGDVAASILTCMPDSEVDVLYGIGGAPEGVVSAAVIRALDGDMNGRLLARHDVKGDNEENRRIGEQELARCKAMGIEAGKVLRLGDMARSDNVIFSATGITKGDLLEGISRKGNIATTETLLIRGKSRTIRRIQSIHYLDRKDPEMQVHIL

SEQ ID NO: 365 - DNA, 999 bp, Escherichia coli
atgaaaacgttaggtgaatttattgtcgaaaagcagcacgagttttctcatgctaccggtgagctcactgctttgctgtcggcaataaaactgggcgccaagattatccatcgcgatatcaacaaagcaggactggttgatatcctgggtgccagcggtgctgagaacgtgcagggcgaggttcagcagaaactcgacttgttcgctaatgaaaaactgaaagccgcactgaaagcacgcgatatcgttgcgggcattgcctctgaagaagaagatgagattgtcgtctttgaaggctgtgaacacgcaaaatacgtggtgctgatggaccccctggatggctcgtccaacatcgatgttaacgtctctgtcggtaccattttctccatctaccgccgcgttacgcctgttggcacgccggtaacggaagaagatttcctccagcctggtaacaaacaggttgcggcaggttacgtggtatacggctcctctaccatgctggtttacaccaccggatgcggtgttcacgcctttacttacgatccttcgctcggcgttttctgcctgtgccaggaacggatgcgcttcccggagaaaggcaaaacctactccatcaacgaaggaaactacattaagtttccgaacggggtgaagaagtacattaaattctgccaggaagaagataaatccaccaaccgcccttatacctcacgttatatcggttcactggtcgcggatttccaccgtaacctgctgaaaggcggtatttatctctacccaagcaccgccagccacccggacggcaaactgcgtttgctgtatgagtgcaacccgatggcattcctggcggaacaagcgggcggtaaagcgagcgatggcaaagagcgtattctggatatcatcccggaaaccctgcaccagcgccgttcattctttgtcggcaacgaccatatggttgaagatgtcgaacgctttatccgtgagttcccggacgcgtaa

SEQ ID NO: 366 - protein, 332 aa, Escherichia coli
MKTLGEFIVEKQHEFSHATGELTALLSAIKLGAKIIHRDINKAGLVDILGASGAENVQGEVQQKLDLFANEKLKAALKARDIVAGIASEEEDEIVVFEGCEHAKYVVLMDPLDGSSNIDVNVSVGTIFSIYRRVTPVGTPVTEEDFLQPGNKQVAAGYVVYGSSTMLVYTTGCGVHAFTYDPSLGVFCLCQERMRFPEKGKTYSINEGNYIKFPNGVKKYIKFCQEEDKSTNRPYTSRYIGSLVADFHRNLLKGGIYLYPSTASHPDGKLRLLYECNPMAFLAEQAGGKASDGKERILDIIPETLHQRRSFFVGNDHMVEDVERFIREFPDA

SEQ ID NO: 367 - DNA, 930 bp, Escherichia coli
atggtacgtatctatacgttgacacttgcgccctctctcgatagcgcaacaattaccccgcaaatttatcccgaaggaaaactgcgctgtaccgcaccggtgttcgaacccgggggcggcggcatcaacgtcgcccgcgccattgcccatcttggaggcagtgccacagcgatcttcccggcgggtggcgcgaccggcgaacacctggtttcactgttggcggatgaaaatgtccccgtcgctactgtagaagccaaagactggacccggcagaatttacacgtacatgtggaagcaagcggtgagcagtatcgttttgttatgccaggcgcggcattaaatgaagatgagtttcgccagcttgaagagcaagttctggaaattgaatccggggccatcctggtcataagcggaagcctgccgccaggtgtgaagctggaaaaattaacccaactgatttccgctgcgcaaaaacaagggatccgctgcatcgtcgacagttctggcgaagcgttaagtgcagcactggcaattggtaacatcgagttggttaagcctaaccaaaaagaactcagtgcgctggtgaatcgcgaactcacccagccggacgatgtccgcaaagccgcgcaggaaatcgttaatagcggcaaggccaaacgggttgtcgtttccctgggtccacaaggagcgctgggtgttgatagtgaaaactgtattcaggtggtgccaccaccggtgaaaagccagagtaccgttggcgctggtgacagcatggtcggcgcgatgacactgaaactggcagaaaatgcctctcttgaagagatggttcgttttggcgtagctgcggggagtgcagccacactcaatcagggaacacgtctgtgctcccatgacgatacgcaaaaaatttacgcttacctttcccgctaa

SEQ ID NO: 368 - protein, 309 aa, Escherichia coli
MVRIYTLTLAPSLDSATITPQIYPEGKLRCTAPVFEPGGGGINVARAIAHLGGSATAIFPAGGATGEHLVSLLADENVPVATVEAKDWTRQNLHVHVEASGEQYRFVMPGAALNEDEFRQLEEQVLEIESGAILVISGSLPPGVKLEKLTQLISAAQKQGIRCIVDSSGEALSAALAIGNIELVKPNQKELSALVNRELTQPDDVRKAAQEIVNSGKAKRVVVSLGPQGALGVDSENCIQVVPPPVKSQSTVGAGDSMVGAMTLKLAENASLEEMVRFGVAAGSAATLNQGTRLCSHDDTQKIYAYLSR

SEQ ID NO: 369 - DNA, 963 bp, Escherichia coli
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 370 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 371 - DNA, 660 bp, Escherichia coli
atgacgcaggatgaattgaaaaaagcagtaggatgggcggcacttcagtatgttcagcccggcaccattgttggtgtaggtacaggttccaccgccgcacactttattgacgcgctcggtacaatgaaaggccagattgaaggggccgtttccagttcagatgcttccactgaaaaactgaaaagcctcggcattcacgtttttgatctcaacgaagtcgacagccttggcatctacgttgatggcgcagatgaaatcaacggccacatgcaaatgatcaaaggcggcggcgcggcgctgacccgtgaaaaaatcattgcttcggttgcagaaaaatttatctgtattgcagacgcttccaagcaggttgatattctgggtaaattcccgctgccagtagaagttatcccgatggcacgtagtgcagtggcgcgtcagctggtgaaactgggcggtcgtccggaataccgtcagggcgtggtgaccgataatggcaacgtgatcctcgacgtccacggcatggaaatccttgacccgatagcgatggaaaacgccataaatgcgattcctggcgtggtgactgttggcttgtttgctaaccgtggcgcggacgttgcgctgattggcacacctgacggtgtcaaaaccattgtgaaatga

SEQ ID NO: 372 - protein, 219 aa, Escherichia coli
MTQDELKKAVGWAALQYVQPGTIVGVGTGSTAAHFIDALGTMKGQIEGAVSSSDASTEKLKSLGIHVFDLNEVDSLGIYVDGADEINGHMQMIKGGGAALTREKIIASVAEKFICIADASKQVDILGKFPLPVEVIPMARSAVARQLVKLGGRPEYRQGVVTDNGNVILDVHGMEILDPIAMENAINAIPGVVTVGLFANRGADVALIGTPDGVKTIVK

SEQ ID NO: 373 - DNA, 450 bp, Escherichia coli
atgaaaaagattgcatttggctgtgatcatgtcggtttcattttaaaacatgaaatagtggcacatttagttgagcgtggcgttgaagtgattgataaaggaacctggtcgtcagagcgtactgattatccacattacgccagtcaagtcgcactggctgttgctggcggagaggttgatggcgggattttgatttgtggtactggcgtcggtatttcgatagcggcgaacaagtttgccggaattcgcgcggtcgtctgtagcgaaccttattccgcgcaactttcgcggcagcataacgacaccaacgtgctggcttttggttcacgagtggttggcctcgaactggcaaaaatgattgtggatgcgtggctgggcgcacagtacgaaggcggtcgtcatcaacaacgcgtggaggcgattacggcaatagagcagcggagaaattga

SEQ ID NO: 374 - protein, 149 aa, Escherichia coli
MKKIAFGCDHVGFILKHEIVAHLVERGVEVIDKGTWSSERTDYPHYASQVALAVAGGEVDGGILICGTGVGISIAANKFAGIRAVVCSEPYSAQLSRQHNDTNVLAFGSRVVGLELAKMIVDAWLGAQYEGGRHQQRVEAITAIEQRRN

SEQ ID NO: 375 - DNA, 954 bp, Escherichia coli
atgacggacaaattgacctcccttcgtcagtacaccaccgtagtggccgacactggggacatcgcggcaatgaagctgtatcaaccgcaggatgccacaaccaacccttctctcattcttaacgcagcgcagattccggaataccgtaagttgattgatgatgctgtcgcctgggcgaaacagcagagcaacgatcgcgcgcagcagatcgtggacgcgaccgacaaactggcagtaaatattggtctggaaatcctgaaactggttccgggccgtatctcaactgaagttgatgcgcgtctttcctatgacaccgaagcgtcaattgcgaaagcaaaacgcctgatcaaactctacaacgatgctggtattagcaacgatcgtattctgatcaaactggcttctacctggcagggtatccgtgctgcagaacagctggaaaaagaaggcatcaactgtaacctgaccctgctgttctccttcgctcaggctcgtgcttgtgcggaagcgggcgtgttcctgatctcgccgtttgttggccgtattcttgactggtacaaagcgaataccgataagaaagagtacgctccggcagaagatccgggcgtggtttctgtatctgaaatctaccagtactacaaagagcacggttatgaaaccgtggttatgggcgcaagcttccgtaacatcggcgaaattctggaactggcaggctgcgaccgtctgaccatcgcaccggcactgctgaaagagctggcggagagcgaaggggctatcgaacgtaaactgtcttacaccggcgaagtgaaagcgcgtccggcgcgtatcactgagtccgagttcctgtggcagcacaaccaggatccaatggcagtagataaactggcggaaggtatccgtaagtttgctattgaccaggaaaaactggaaaaaatgatcggcgatctgctgtaa

SEQ ID NO: 376 - protein, 317 aa, Escherichia coli
MTDKLTSLRQYTTVVADTGDIAAMKLYQPQDATTNPSLILNAAQIPEYRKLIDDAVAWAKQQSNDRAQQIVDATDKLAVNIGLEILKLVPGRISTEVDARLSYDTEASIAKAKRLIKLYNDAGISNDRILIKLASTWQGIRAAEQLEKEGINCNLTLLFSFAQARACAEAGVFLISPFVGRILDWYKANTDKKEYAPAEDPGVVSVSEIYQYYKEHGYETVVMGASFRNIGEILELAGCDRLTIAPALLKELAESEGAIERKLSYTGEVKARPARITESEFLWQHNQDPMAVDKLAEGIRKFAIDQEKLEKMIGDLL

SEQ ID NO: 377 - DNA, 951 bp, Escherichia coli
atgaacgagttagacggcatcaaacagttcaccactgtcgtggcagacagcggcgatattgagtccattcgccattatcatccccaggatgccaccaccaatccttcgctgttactcaaagctgccggattatcacaatatgagcatttaatagacgatgctatcgcctggggtaaaaaaaatggcaagacccaggaacaacaggtggtcgcagcgtgtgacaaactggcggtcaatttcggtgctgaaatcctcaaaatcgtacccggtcgcgtgtcaacagaagttgatgcacgcctctcttttgataaagaaaagagtattgagaaggcgcgccatctggtggacttgtatcagcaacaaggcgttgagaaatcacgcattctgatcaagctggcttcgacctgggaaggaattcgcgcggcagaagagctggaaaaagaaggtattaactgcaacctgacgctgctgttttcttttgcacaggcacgggcctgtgcggaagcaggcgtttttctgatttcgccgtttgtcgggcgtatttatgactggtatcaggcacgcaagccgatggacccgtatgtggtggaagaagatccgggcgttaaatcggtgcgcaatatctacgactactataagcaacaccactatgaaaccattgtgatgggcgcgagcttccgtcgcaccgaacaaatcctcgccttaaccggctgcgatcgactgactatcgcaccgaatttactgaaggagctgcaggaaaaagtttcgccagtggtacgtaaattaatcccaccttctcagacgttcccacgcccagctcccatgagcgaagcggagttccgttgggagcacaatcaggatgcgatggcggtagaaaaactgtctgaaggcattcgtctgttcgccgttgatcaacgcaaactggaagatcttcttgccgccaaactataa

SEQ ID NO: 378 - protein, 316 aa, Escherichia coli
MNELDGIKQFTTVVADSGDIESIRHYHPQDATTNPSLLLKAAGLSQYEHLIDDAIAWGKKNGKTQEQQVVAACDKLAVNFGAEILKIVPGRVSTEVDARLSFDKEKSIEKARHLVDLYQQQGVEKSRILIKLASTWEGIRAAEELEKEGINCNLTLLFSFAQARACAEAGVFLISPFVGRIYDWYQARKPMDPYVVEEDPGVKSVRNIYDYYKQHHYETIVMGASFRRTEQILALTGCDRLTIAPNLLKELQEKVSPVVRKLIPPSQTFPRPAPMSEAEFRWEHNQDAMAVEKLSEGIRLFAVDQRKLEDLLAAKL

SEQ ID NO: 379 - DNA, 930 bp, Escherichia coli
atgcaaaacgcaggcagcctcgttgttcttggcagcattaatgctgaccacattcttaatcttcaatcttttcctactccaggcgaaaccgtaaccggtaaccactatcaggttgcatttggcggcaaaggcgcgaatcaggctgtggctgctgggcgtagcggtgcgaatatcgcgtttattgcctgtacgggtgatgacagcattggtgagagcgttcgccagcagctcgccactgataacattgatattactccggtcagcgtgatcaaaggcgaatcaacaggtgtggcgctgatttttgttaatggcgaaggtgagaatgtcatcggtattcatgccggcgctaatgctgccctttccccggcgctggtggaagcgcaacgtgagcgtattgccaacgcgtcagcattattaatgcagctggaatcaccactcgaaagtgtgatggcagcggcgaaaatcgcccatcaaaataagactatcgttgcgcttaacccggctccggctcgcgaacttcctgacgaactgctggcgctggtggacattattacgccaaacgaaacggaagcagaaaagctcaccggtattcgtgttgaaaatgatgaagatgcagcgaaggcggcgcaggtactgcatgaaaaaggtatccgtactgtactgattactttaggaagtcgtggtgtatgggctagcgtgaatggtgaaggtcagcgcgttcctggattccgggtgcaggctgtcgataccattgctgccggagatacctttaacggtgcgttaatcacggcattgctggaagaaaaaccattgccagaggcgattcgttttgcccatgctgccgctgcgattgccgtaacacgtaaaggcgcacaaccttccgtaccgtggcgtgaagagatcgacgcatttttagacaggcagaggtga

SEQ ID NO: 380 - protein, 309 aa, Escherichia coli
MQNAGSLVVLGSINADHILNLQSFPTPGETVTGNHYQVAFGGKGANQAVAAGRSGANIAFIACTGDDSIGESVRQQLATDNIDITPVSVIKGESTGVALIFVNGEGENVIGIHAGANAALSPALVEAQRERIANASALLMQLESPLESVMAAAKIAHQNKTIVALNPAPARELPDELLALVDIITPNETEAEKLTGIRVENDEDAAKAAQVLHEKGIRTVLITLGSRGVWASVNGEGQRVPGFRVQAVDTIAAGDTFNGALITALLEEKPLPEAIRFAHAAAAIAVTRKGAQPSVPWREEIDAFLDRQR

SEQ ID NO: 381 - DNA, 1224 bp, Escherichia coli
atgaaacgtgcatttattatggtgctggactcattcggcatcggcgctacagaagatgcagaacgctttggtgacgtcggggctgacaccctgggtcatatcgcagaagcttgtgccaaaggcgaagctgataacggtcgtaaaggcccgctcaatctgccaaatctgacccgtctggggctggcgaaagcacacgaaggttctaccggtttcattccggcgggaatggacggcaacgctgaagttatcggcgcgtacgcatgggcgcacgaaatgtcatccggtaaagataccccgtctggtcactgggaaattgccggtgtcccggttctgtttgagtggggatatttctccgatcacgaaaacagcttcccgcaagagctgctggataaactggtcgaacgcgctaatctgccgggttacctcggtaactgccactcttccggtacggtcattctggatcaactgggcgaagagcacatgaaaaccggcaagccgattttctatacctccgctgactccgtgttccagattgcctgccatgaagaaactttcggtctggataaactctacgaactgtgcgaaatcgcccgtgaagagctgaccaacggcggctacaatatcggtcgtgttatcgctcgtccgtttatcggcgacaaagccggtaacttccagcgtaccggtaaccgtcacgacctggctgttgagccgccagcaccgaccgtgctgcagaaactggttgatgaaaaacacggccaggtggtttctgtcggtaaaattgcggacatctacgccaactgcggtatcaccaaaaaagtgaaagcgactggcctggacgcgctgtttgacgccaccatcaaagagatgaaagaagcgggtgataacaccatcgtcttcaccaacttcgttgacttcgactcttcctggggccaccgtcgcgacgtcgccggttatgccgcgggtctggaactgttcgaccgccgtctgccggagctgatgtctctgctgcgcgatgacgacatcctgatcctcaccgctgaccacggttgcgatccgacctggaccggtactgaccacacgcgtgaacacattccggtactggtatatggcccgaaagtaaaaccgggctcactgggtcatcgtgaaaccttcgcggatatcggccagactctggcaaaatattttggtacttctgatatggaatatggcaaagccatgttctga

SEQ ID NO: 382 - protein, 407 aa, Escherichia coli
MKRAFIMVLDSFGIGATEDAERFGDVGADTLGHIAEACAKGEADNGRKGPLNLPNLTRLGLAKAHEGSTGFIPAGMDGNAEVIGAYAWAHEMSSGKDTPSGHWEIAGVPVLFEWGYFSDHENSFPQELLDKLVERANLPGYLGNCHSSGTVILDQLGEEHMKTGKPIFYTSADSVFQIACHEETFGLDKLYELCEIAREELTNGGYNIGRVIARPFIGDKAGNFQRTGNRHDLAVEPPAPTVLQKLVDEKHGQVVSVGKIADIYANCGITKKVKATGLDALFDATIKEMKEAGDNTIVFTNFVDFDSSWGHRRDVAGYAAGLELFDRRLPELMSLLRDDDILILTADHGCDPTWTGTDHTREHIPVLVYGPKVKPGSLGHRETFADIGQTLAKYFGTSDMEYGKAMF

SEQ ID NO: 383 - DNA, 1641 bp, Escherichia coli
atggcaatccacaatcgtgcaggccaacctgcacaacagagtgatttgattaacgtcgcccaactgacggcgcaatattatgtactgaaaccagaagcagggaatgcggagcacgcggtgaaattcggtacttccggtcaccgtggcagtgcagcgcgccacagctttaacgagccgcacattctggcgatcgctcaggcaattgctgaagaacgtgcgaaaaacggcatcactggcccttgctatgtgggtaaagatactcacgccctgtccgaacctgcattcatttccgttctggaagtgctggcagcgaacggcgttgatgtcattgtgcaggaaaacaatggcttcaccccgacgcctgccgtttccaatgccatcctggttcacaataaaaaaggtggcccgctggcagacggtatcgtgattacaccgtcccataacccgccggaagatggtggaatcaaatacaatccgccaaatggtggcccggctgataccaacgtcactaaagtggtggaagacagggccaacgcactgctggccgatggcctgaaaggcgtgaagcgtatctccctcgacgaagcgatggcatccggtcatgtgaaagagcaggatctggtgcagccgttcgtggaaggtctggccgatatcgttgatatggccgcgattcagaaagcgggcctgacgctgggcgttgatccgctgggcggttccggtatcgaatactggaagcgtattggcgagtattacaacctcaacctgactatcgttaacgatcaggtcgatcaaaccttccgctttatgcaccttgataaagacggcgcgatccgtatggactgctcctccgagtgtgcgatggcgggcctgctggcactgcgtgataagttcgatctggcgtttgctaacgacccggattatgaccgtcacggtatcgtcactccggcaggtttgatgaatccgaaccactacctggcggtggcaatcaattacctgttccagcatcgtccgcagtggggcaaagatgttgccgtcggtaaaacgctggtttcatctgcgatgatcgaccgtgtggtcaacgacttgggccgtaaactggtagaagtcccggtaggtttcaaatggtttgtcgatggtctgttcgacggcagcttcggctttggcggcgaagagagtgcaggggcttccttcctgcgtttcgacggcacgccgtggtccaccgacaaagacggcatcatcatgtgtctgctggcggcggaaatcaccgctgtcaccggtaagaacccgcaggaacactacaacgaactggcaaaacgctttggtgcgccgagctacaaccgtttgcaggcagctgcgacttccgcacaaaaagcggcgctgtctaagctgtctccggaaatggtgagcgccagcaccctggcaggtgacccgatcaccgcgcgcctgactgctgctccgggcaacggtgcttctattggcggtctgaaagtgatgactgacaacggctggttcgccgcgcgtccgtcaggcacggaagacgcatataagatctactgcgaaagcttcctcggtgaagaacatcgcaagcagattgagaaagaagcggttgagattgttagcgaagttctgaaaaacgcgtaa

SEQ ID NO: 384 - protein, 546 aa, Escherichia coli
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 385 - DNA, 558 bp, Escherichia coli
atgatgggaaaactgatttggttaatggggccgtccggctccgggaaagacagcctgctggcggaactccgcctgcgggaacaaactcagttactggtggcgcatcgctacatcacgcgcgatgccagcgccggaagtgaaaaccatatcgccctgagcgagcaggagttttttacccgcgcggggcaaaatctgttggccttaagctggcacgctaacggtctgtattatggcgtcggcgtcgagattgatctctggctgcacgccggattcgacgtgctggtcaacggctcacgcgcccatctgccgcaggcgcgggcgcgctatcaatcggcgctgctgcccgtctgtttacaggtttcgccggagatcctccgccagcgcctggaaaaccgtggccgtgaaaacgccagtgaaattaacgcccgcctggcgcgcgccgcccgctatactccacaggattgccatacgctcaacaatgacggcagcctgcgccagtcggtcgacacgctgctgacgctgatccatcagaaggagaaacaccatgcctgcttgtga

SEQ ID NO: 386 - protein, 185 aa, Escherichia coli [sequence omitted]

SEQ ID NO: 387 - DNA, 948 bp, Escherichia coli
gtgcctgatatgaagctttttgctggtaacgccaccccggaactagcacaacgtattgccaaccgcctgtacacttcactcggcgacgccgctgtaggtcgctttagcgatggcgaagtcagcgtacaaattaatgaaaatgtacgcggtggtgatattttcatcatccagtccacttgtgcccctactaacgacaacctgatggaattagtcgttatggttgatgccctgcgtcgtgcttccgcaggtcgtatcaccgctgttatcccctactttggctatgcgcgccaggaccgtcgcgtccgttccgctcgtgtaccaatcactgcgaaagtggttgcagacttcctctccagcgtcggtgttgaccgtgtgctgacagtggatctgcacgctgaacagattcagggtttcttcgacgttccggttgataacgtatttggtagcccgatcctgctggaagacatgctgcagctgaatctggataacccaattgtggtttctccggacatcggcggcgttgtgcgtgcccgcgctatcgctaagctgctgaacgataccgatatggcaatcatcgacaaacgtcgtccgcgtgcgaacgtttcacaggtgatgcatatcatcggtgacgttgcaggtcgtgactgcgtactggtcgatgatatgatcgacactggcggtacgctgtgtaaagctgctgaagctctgaaagaacgtggtgctaaacgtgtatttgcgtacgcgactcacccgatcttctctggcaacgcggcgaacaacctgcgtaactctgtaattgatgaagtcgttgtctgcgataccattccgctgagcgatgaaatcaaatcactgccgaacgtgcgtactctgaccctgtcaggtatgctggccgaagcgattcgtcgtatcagcaacgaagaatcgatctctgccatgttcgaacactaa

SEQ ID NO: 388 - protein, 315 aa, Escherichia coli
MPDMKLFAGNATPELAQRIANRLYTSLGDAAVGRFSDGEVSVQINENVRGGDIFIIQSTCAPTNDNLMELVVMVDALRRASAGRITAVIPYFGYARQDRRVRSARVPITAKVVADFLSSVGVDRVLTVDLHAEQIQGFFDVPVDNVFGSPILLEDMLQLNLDNPIVVSPDIGGVVRARAIAKLLNDTDMAIIDKRRPRANVSQVMHIIGDVAGRDCVLVDDMIDTGGTLCKAAEALKERGAKRVFAYATHPIFSGNAANNLRNSVIDEVVVCDTIPLSDEIKSLPNVRTLTLSGMLAEAIRRISNEESISAMFEH

SEQ ID NO: 389 - DNA, 1053 bp, Escherichia coli
atgacagatattgcgcagttgcttggcaaagacgccgacaaccttttacagcaccgttgtatgacaattccttctgaccagctttatctccccggacatgactacgtagaccgcgtaatgattgacaataatcgcccgccagcggtgttacgtaatatgcagacgttgtacaacaccgggcgtctggctggcacaggatatctttctattctgccggttgaccagggcgttgagcactctgccggagcttcatttgctgctaacccgctctactttgacccgaaaaacattgttgaactggcgatcgaagcgggctgtaactgtgtggcgtcaacttacggcgtgctggcgtcggtatcgcggcgttatgcgcatcgcattccattcctcgtcaaacttaatcacaacgagacgctaagttacccgaatacctacgatcaaacgctgtatgccagcgtggagcaggcgttcaacatgggcgcggttgcggttggtgcgactatctattttggctcggaagagtcacgtcgccagattgaagaaatttctgcggcttttgaacgtgcgcacgagctgggtatggtgacagtgctgtgggcctatttgcgtaactccgcctttaagaaagatggcgttgattaccatgtttccgccgacctgaccggtcaggcaaaccatctggcggcaaccatcggtgcagatatcgtcaaacaaaaaatggcggaaaataacggcggctataaagcaattaattacggttacaccgacgatcgtgtttacagcaaattgaccagcgaaaacccgattgatctggtgcgttatcagttagctaactgctatatgggtcgggctgggttgataaactccggcggtgctgcgggcggtgaaactgacctcagcgatgcagtgcgtactgcggttatcaacaaacgcgcaggcggaatggggctgattcttggacgtaaagcgttcaagaaatcgatggctgacggcgtgaaactgattaacgccgtgcaggacgtttatctcgatagcaaaattactatcgcctga

SEQ ID NO: 390 - protein, 350 aa, Escherichia coli [sequence omitted]

SEQ ID NO: 391 - DNA, 1080 bp, Escherichia coli
atgtctaagatttttgatttcgtaaaacctggcgtaatcactggtgatgacgtacagaaagttttccaggtagcaaaagaaaacaacttcgcactgccagcagtaaactgcgtcggtactgactccatcaacgccgtactggaaaccgctgctaaagttaaagcgccggttatcgttcagttctccaacggtggtgcttcctttatcgctggtaaaggcgtgaaatctgacgttccgcagggtgctgctatcctgggcgcgatctctggtgcgcatcacgttcaccagatggctgaacattatggtgttccggttatcctgcacactgaccactgcgcgaagaaactgctgccgtggatcgacggtctgttggacgcgggtgaaaaacacttcgcagctaccggtaagccgctgttctcttctcacatgatcgacctgtctgaagaatctctgcaagagaacatcgaaatctgctctaaatacctggagcgcatgtccaaaatcggcatgactctggaaatcgaactgggttgcaccggtggtgaagaagacggcgtggacaacagccacatggacgcttctgcactgtacacccagccggaagacgttgattacgcatacaccgaactgagcaaaatcagcccgcgtttcaccatcgcagcgtccttcggtaacgtacacggtgtttacaagccgggtaacgtggttctgactccgaccatcctgcgtgattctcaggaatatgtttccaagaaacacaacctgccgcacaacagcctgaacttcgtattccacggtggttccggttctactgctcaggaaatcaaagactccgtaagctacggcgtagtaaaaatgaacatcgataccgatacccaatgggcaacctgggaaggcgttctgaactactacaaagcgaacgaagcttatctgcagggtcagctgggtaacccgaaaggcgaagatcagccgaacaagaaatactacgatccgcgcgtatggctgcgtgccggtcagacttcgatgatcgctcgtctggagaaagcattccaggaactgaacgcgatcgacgttctgtaa

SEQ ID NO: 392 - protein, 359 aa, Escherichia coli
MSKIFDFVKPGVITGDDVQKVFQVAKENNFALPAVNCVGTDSINAVLETAAKVKAPVIVQFSNGGASFIAGKGVKSDVPQGAAILGAISGAHHVHQMAEHYGVPVILHTDHCAKKLLPWIDGLLDAGEKHFAATGKPLFSSHMIDLSEESLQENIEICSKYLERMSKIGMTLEIELGCTGGEEDGVDNSHMDASALYTQPEDVDYAYTELSKISPRFTIAASFGNVHGVYKPGNVVLTPTILRDSQEYVSKKHNLPHNSLNFVFHGGSGSTAQEIKDSVSYGVVKMNIDTDTQWATWEGVLNYYKANEAYLQGQLGNPKGEDQPNKKYYDPRVWLRAGQTSMIARLEKAFQELNAIDVL

SEQ ID NO: 393 - DNA, 780 bp, Escherichia coli
atgactgatctgaaagcaagcagcctgcgtgcactgaaattgatggacctgaccaccctgaatgacgacgacaccgacgagaaagtgatcgccctgtgtcatcaggccaaaactccggtcggcaataccgccgctatctgtatctatcctcgctttatcccgattgctcgcaaaactctgaaagagcagggcaccccggaaatccgtatcgctacggtaaccaacttcccacacggtaacgacgacatcgacatcgcgctggcagaaacccgtgcggcaatcgcctacggtgctgatgaagttgacgttgtgttcccgtaccgcgcgctgatggcgggtaacgagcaggttggttttgacctggtgaaagcctgtaaagaggcttgcgcggcagcgaatgtactgctgaaagtgatcatcgaaaccggcgaactgaaagacgaagcgctgatccgtaaagcgtctgaaatctccatcaaagcgggtgcggacttcatcaaaacctctaccggtaaagtggctgtgaacgcgacgccggaaagcgcgcgcatcatgatggaagtgatccgtgatatgggcgtagaaaaaaccgttggtttcaaaccggcgggcggcgtgcgtactgcggaagatgcgcagaaatatctcgccattgcagatgaactgttcggtgctgactgggcagatgcgcgtcactaccgctttggcgcttccagcctgctggcaagcctgctgaaagcgctgggtcacggcgacggtaagagcgccagcagctactaa

SEQ ID NO: 394 - protein, 259 aa, Escherichia coli
MTDLKASSLRALKLMDLTTLNDDDTDEKVIALCHQAKTPVGNTAAICIYPRFIPIARKTLKEQGTPEIRIATVTNFPHGNDDIDIALAETRAAIAYGADEVDVVFPYRALMAGNEQVGFDLVKACKEACAAANVLLKVIIETGELKDEALIRKASEISIKAGADFIKTSTGKVAVNATPESARIMMEVIRDMGVEKTVGFKPAGGVRTAEDAQKYLAIADELFGADWADARHYRFGASSLLASLLKALGHGDGKSASSY

SEQ ID NO: 395 - DNA, 582 bp, Saccharomyces cerevisiae
atgacagagaaacacaaaactatggggaaatttaaggttattgtattggcaggtactgcaggtacaggaaaatcaaccattgcaggtgaactaatccatgaatttaaagatatatacccggatttgaaattcatcgaaggtgatgatttacacccgcctgccaacgtagaaaagatgacaagagggattccattaaatgacgacgaccgttgggactggttgaaaaaagttgccgtagaatctacaaaagctgcagcaagcactaaggagcatttgtcaattgttgcttgctcgagcttgaagaagaaatacagagatttgattaggcacacatgccctgaatcggagttccattttatctttttgtatgcaagtaaaatagaagttctgaaaaggctcaaaacaaggaagggtcatttcatgaaagctgatatgatggagtctcaattcagggatctggagttaccagacatcaacgacgaaactgactgtgacattgttcccttagatttcaaaacgttttatcaaattgaaaaagatgttatacaggtggtaaaaagtaaagttttgaatattgagtaa

SEQ ID NO: 396 - protein, 193 aa, Saccharomyces cerevisiae
MTEKHKTMGKFKVIVLAGTAGTGKSTIAGELIHEFKDIYPDLKFIEGDDLHPPANVEKMTRGIPLNDDDRWDWLKKVAVESTKAAASTKEHLSIVACSSLKKKYRDLIRHTCPESEFHFIFLYASKIEVLKRLKTRKGHFMKADMMESQFRDLELPDINDETDCDIVPLDFKTFYQIEKDVIQVVKSKVLNIE

SEQ ID NO: 397 - DNA, 1665 bp, Saccharomyces cerevisiae
atgtccaataactcattcactaacttcaaactggccactgaattgccagcctggtctaagttgcaaaaaatttatgaatctcaaggtaagactttgtctgtcaagcaagaattccaaaaagatgccaagcgttttgaaaaattgaacaagactttcaccaactatgatggttccaaaatcttgttcgactactcaaagaacttggtcaacgatgaaatcattgctgcattgattgaactggccaaggaggctaacgtcaccggtttgagagatgctatgttcaaaggtgaacacatcaactccactgaagatcgtgctgtctaccacgtcgcattgagaaacagagctaacaagccaatgtacgttgatggtgtcaacgttgctccagaagtcgactctgtcttgaagcacatgaaggagttctctgaacaagttcgttctggtgaatggaagggttataccggtaagaagatcaccgatgttgttaacatcggtattggtggttccgatttgggtccagtcatggtcactgaggctttgaagcactacgctggtgtcttggatgtccacttcgtttccaacattgacggtactcacattgctgaaaccttgaaggttgttgacccagaaactactttgtttttgattgcttccaagactttcactaccgctgaaactatcactaacgctaacactgccaagaactggttcttgtcgaagacaggtaatgatccatctcacattgctaagcatttcgctgctttgtccactaacgaaaccgaagttgccaagttcggtattgacaccaaaaacatgtttggtttcgaaagttgggtcggtggtcgttactctgtctggtcggctattggtttgtctgttgccttgtacattggctatgacaactttgaggctttcttgaagggtgctgaagccgtcgacaaccacttcacccaaaccccattggaagacaacattccattgttgggtggtttgttgtctgtctggtacaacaacttctttggtgctcaaacccatttggttgctccattcgaccaatacttgcacagattcccagcctacttgcaacaattgtcaatggaatctaacggtaagtctgttaccagaggtaacgtgtttactgactactctactggttctatcttgtttggtgaaccagctaccaacgctcaacactctttcttccaattggttcaccaaggtaccaagttgattccatctgatttcatcttagctgctcaatctcataacccaattgagaacaaattacatcaaaagatgttggcttcaaacttctttgctcaagctgaagctttaatggttggtaaggatgaagaacaagttaaggctgaaggtgccactggtggtttggtcccacacaaggtcttctcaggtaacagaccaactacctctatcttggctcaaaagattactccagctactttgggtgctttgattgcctactacgaacatgttactttcactgaaggtgccatttggaatatcaactctttcgaccaatggggtgttgaattgggtaaagtcttggctaaagtcatcggcaaggaattggacaactcctccaccatttctacccacgatgcttctaccaacggtttaatcaatcaattcaaggaatggatgtga

SEQ ID NO: 398 - protein, 554 aa, Saccharomyces cerevisiae
MSNNSFTNFKLATELPAWSKLQKIYESQGKTLSVKQEFQKDAKRFEKLNKTFTNYDGSKILFDYSKNLVNDEIIAALIELAKEANVTGLRDAMFKGEHINSTEDRAVYHVALRNRANKPMYVDGVNVAPEVDSVLKHMKEFSEQVRSGEWKGYTGKKITDVVNIGIGGSDLGPVMVTEALKHYAGVLDVHFVSNIDGTHIAETLKVVDPETTLFLIASKTFTTAETITNANTAKNWFLSKTGNDPSHIAKHFAALSTNETEVAKFGIDTKNMFGFESWVGGRYSVWSAIGLSVALYIGYDNFEAFLKGAEAVDNHFTQTPLEDNIPLLGGLLSVWYNNFFGAQTHLVAPFDQYLHRFPAYLQQLSMESNGKSVTRGNVFTDYSTGSILFGEPATNAQHSFFQLVHQGTKLIPSDFILAAQSHNPIENKLHQKMLASNFFAQAEALMVGKDEEQVKAEGATGGLVPHKVFSGNRPTTSILAQKITPATLGALIAYYEHVTFTEGAIWNINSFDQWGVELGKVLAKVIGKELDNSSTISTHDASTNGLINQFKEWM

SEQ ID NO: 399 - DNA, 1518 bp, Saccharomyces cerevisiae
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 400 - protein, 505 aa, Saccharomyces cerevisiae
MSEGPVKFEKNTVISVFGASGDLAKKKTFPALFGLFREGYLDPSTKIFGYARSKLSMEEDLKSRVLPHLKKPHGEADDSKVEQFFKMVSYISGNYDTDEGFDELRTQIEKFEKSANVDVPHRLFYLALPPSVFLTVAKQIKSRVYAENGITRVIVEKPFGHDLASARELQKNLGPLFKEEELYRIDHYLGKELVKNLLVLRFGNQFLNASWNRDNIQSVQISFKERFGTEGRGGYFDSIGIIRDVMQNHLLQIMTLLTMERPVSFDPESIRDEKVKVLKAVAPIDTDDVLLGQYGKSEDGSKPAYVDDDTVDKDSKCVTFAAMTFNIENERWEGVPIMMRAGKALNESKVEIRLQYKAVASGVFKDIPNNELVIRVQPDAAVYLKFNAKTPGLSNATQVTDLNLTYASRYQDFWIPEAYEVLIRDALLGDHSNFVRDDELDISWGIFTPLLKHIERPDGPTPEIYPYGSRGPKGLKEYMQKHKYVMPEKHPYAWPVTKPEDTKDN

SEQ ID NO: 401 - DNA, 948 bp, Saccharomyces cerevisiae
atgactacgacggtacccaagatattcgcgtttcacgagttttcagacgtggcagaggccgtagctgaccatgtagtccacgcgcaagacggtgcattggctccaaagaacgagaggaaacactctgttcccaacatcagcatgaatgcactggatatgacgagagaggcctcttgcaaaagcacagcatctgccgcggaagggaaaagtggtagcagtggtagtggcagtggtagcagtaagcccaaaaaggagaaacggttcaagattgctctctccggtgggtcattgatcgaagtgctacacgaaggtctgctaaaacgagacgatgtacggtggggagactgggacatttactttgcagacgagagacttgtacccttcagctcgaatgaaagcaattatggatgcgccaaaaggaagattttggacctgatagacacggcgaagtatggaactccgaaggtgtaccacattgacgagtcattgattgacgacccgcaagaatgcgctgataactatgaaaaggtgctaatccgcgggtttgccggtagagattccgtcaaacttccgatgttcgacttgttcctgcttggttgtgcccccgatggtcatatcgcatcactcttccctaacttccaggacaatctacgtgagaaacttgcatgggtggtgcccgtggagaacgctcctagtgggccctcgaccagaatttcgctgactatacctgtaatctgccattctcacagggttactttcgttgtcgaaggtgcaaccaaggcgcccatcatcaagaccattatggaaaggcctgaaaagggcctacctagcagtattgtcaacgaaggtgctgctggtcgtgtatcatggtttgttgacgacgatgctcttacggacgtcctcgtcaccaaaaaaaagtataaattccaccaaggtttgtctatttaa

SEQ ID NO: 402 - protein, 315 aa, Saccharomyces cerevisiae
MTTTVPKIFAFHEFSDVAEAVADHVVHAQDGALAPKNERKHSVPNISMNALDMTREASCKSTASAAEGKSGSSGSGSGSSKPKKEKRFKIALSGGSLIEVLHEGLLKRDDVRWGDWDIYFADERLVPFSSNESNYGCAKRKILDLIDTAKYGTPKVYHIDESLIDDPQECADNYEKVLIRGFAGRDSVKLPMFDLFLLGCAPDGHIASLFPNFQDNLREKLAWVVPVENAPSGPSTRISLTIPVICHSHRVTFVVEGATKAPIIKTIMERPEKGLPSSIVNEGAAGRVSWFVDDDALTDVLVTKKKYKFHQGLSI

SEQ ID NO: 403 - DNA, 768 bp, Saccharomyces cerevisiae
atggtgaaattacaaaggtttagcgaaaagaaaagcctcatacacgaattcggcaagtttatccttgaaaagcaagaatcggcgttaacgggcgacgctgatgcagtgttcaatatcgccatcagtggaggatcgatgaaccaagcgctgtacgaaagtttggtaaatgacaaaaacatttttccacatattaagtggccacaatggagaatcttcttctgtgacgaaagattggttccatttgaggatccgcaaagtaactatggtcagttcaaaaaaacagttttggacccgctagtgcatcagggcaaccaattgaacttaggccccactgtatacactatcaacgaatcattaatcggtggcggtgaaacggccaatagaaagattgccgaagaatacgcttccatgctgcctgcatcattcgacctaatcttactcggatgcggagaagatggacatacatgctcgttgtttcccggggttgaatttaattaccttgtagaagagatggaccgcaaggttttatggtgtaataattcgcccaaggcacccaaggacaggatcacctttacattagcagtagtagccgaggctaaaagtgtgtgctttctcgttaggggagctgctaaaaaggctatcatgcatgacgtgttaatcgtaaaaaatagcgaactacctagtgtgctggttaatgaaatggtcggaaccaaagtaacttggtttctcgacgacgaagctggcgccttgattcctgaaaactgctaa

SEQ ID NO: 404 - protein, 255 aa, Saccharomyces cerevisiae
MVKLQRFSEKKSLIHEFGKFILEKQESALTGDADAVFNIAISGGSMNQALYESLVNDKNIFPHIKWPQWRIFFCDERLVPFEDPQSNYGQFKKTVLDPLVHQGNQLNLGPTVYTINESLIGGGETANRKIAEEYASMLPASFDLILLGCGEDGHTCSLFPGVEFNYLVEEMDRKVLWCNNSPKAPKDRITFTLAVVAEAKSVCFLVRGAAKKAIMHDVLIVKNSELPSVLVNEMVGTKVTWFLDDEAGALIPENC

SEQ ID NO: 405 - DNA, 768 bp, Saccharomyces cerevisiae; identical to SEQ ID NO: 403

SEQ ID NO: 406 - protein, 255 aa, Saccharomyces cerevisiae; identical to SEQ ID NO: 404

SEQ ID NO: 407 - DNA, 750 bp, Saccharomyces cerevisiae
atggtgacagtcggtgtgttttctgagagggctagtttgacccatcaattgggggaattcatcgtcaagaaacaagatgaggcgctgcaaaagaagtcagactttaaagtttccgttagcggtggctctttgatcgatgctctgtatgaaagtttagtagcggacgaatcactatcttctcgagtgcaatggtctaaatggcaaatctacttctctgatgaaagaattgtgccactgacggacgctgacagcaattatggtgccttcaagagagctgttctagataaattaccctcgactagtcagccaaacgtttatcccatggacgagtccttgattggcagcgatgctgaatctaacaacaaaattgctgcagagtacgagcgtatcgtacctcaagtgcttgatttggtactgttgggctgtggtcctgatggacacacttgttccttattccctggagaaacacataggtacttgctgaacgaaacaaccaaaagagttgcttggtgccacgattctcccaagcctccaagtgacagaatcaccttcactctgcctgtgttgaaagacgccaaagccctgtgttttgtggctgagggcagttccaaacaaaatataatgcatgagatctttgacttgaaaaacgatcaattgccaaccgcattggttaacaaattatttggtgaaaaaacatcctggttcgttaatgaggaagcttttggaaaagttcaaacgaaaactttttag

SEQ ID NO: 408 - protein, 249 aa, Saccharomyces cerevisiae
MVTVGVFSERASLTHQLGEFIVKKQDEALQKKSDFKVSVSGGSLIDALYESLVADESLSSRVQWSKWQIYFSDERIVPLTDADSNYGAFKRAVLDKLPSTSQPNVYPMDESLIGSDAESNNKIAAEYERIVPQVLDLVLLGCGPDGHTCSLFPGETHRYLLNETTKRVAWCHDSPKPPSDRITFTLPVLKDAKALCFVAEGSSKQNIMHEIFDLKNDQLPTALVNKLFGEKTSWFVNEEAFGKVQTKTF

SEQ ID NO: 409 - DNA, 966 bp, Saccharomyces cerevisiae
atgacaacaaccgtacctaaagtgtttgcattccatgaatttgcaggagtagctgaggcagttgccgatcatgttattcacgcacaaaactctgcccttaagaagggcaaagtctcaaggtccacacagatgtccggaacgagccttaacggaaatggtaacacagaatccaaaacgatggagagagtaaattctgtaaggagtaatgcctccagccgtggtggtagtgaagacggcgccactaagaaattgaaaaaggagaaagaaaggcgcttcaagattgcgttgtccggtggttcgttaatccaagtgttgcacgaaggattgttgaaaagagacgacgttcaatggggtaaatgggacatttactttgctgatgaaagactagtgccctttagctccagtgaaagcaattacggtcttgctaagagaaaaatattcgatttaatagatacggagaagtacggaactccgaaaatttatcacattgatgagtcattgattaacgaccctcaagagtgtgcagataactatgagaaaattctaattaaaggatttgccggtagagattctgtgaaactaccgatgtttgatctgtttctgcttggctgcgctcctgacggtcacatagcctcgttattccccaattttcaagagaatctacgtgaaaatctggcatgggtcatacctgtagaaaatgcacccagcggaccttcgaataggatttcactgaccatcccagtgatttgtcactctcatagagtcacctttgttgtagagggcgccacaaaggcgcctgtaatcaagacaataatggaaaggcctgaaaagggattgcctagtagtattgtcaatgagggcgccgccggtcgtgtttcatggtttgtagacgacgatgctttgaaagatgtctttgtaattaagaagaagtacaagttttatgatgatgagaatttgactgagtag

SEQ ID NO: 410 - protein, 321 aa, Saccharomyces cerevisiae
MTTTVPKVFAFHEFAGVAEAVADHVIHAQNSALKKGKVSRSTQMSGTSLNGNGNTESKTMERVNSVRSNASSRGGSEDGATKKLKKEKERRFKIALSGGSLIQVLHEGLLKRDDVQWGKWDIYFADERLVPFSSSESNYGLAKRKIFDLIDTEKYGTPKIYHIDESLINDPQECADNYEKILIKGFAGRDSVKLPMFDLFLLGCAPDGHIASLFPNFQENLRENLAWVIPVENAPSGPSNRISLTIPVICHSHRVTFVVEGATKAPVIKTIMERPEKGLPSSIVNEGAAGRVSWFVDDDALKDVFVIKKKYKFYDDENLTE

SEQ ID NO: 411 - DNA, 1665 bp, Saccharomyces cerevisiae; identical to SEQ ID NO: 397

SEQ ID NO: 412 - protein, 554 aa, Saccharomyces cerevisiae; identical to SEQ ID NO: 398

SEQ ID NO: 413 - DNA, 1479 bp, Saccharomyces cerevisiae
atgtcaaaggcagtaggtgatttaggcttagttggtttagccgtgatgggtcaaaatttgatcttaaacgcagcggatcacggatttaccgtggttgcttataataggacgcaatcaaaggtagataggtttctagctaatgaggcaaaaggaaaatcaataattggtgcaacttcaattgaggacttggttgcgaaactaaagaaacctagaaagattatgcttttaatcaaagccggtgctccggtcgacactttaataaaggaacttgtaccacatcttgataaaggcgacattattatcgacggtggtaactcacatttcccggacactaacagacgctacgaagagctaacaaagcaaggaattctttttgtgggctctggtgtctcaggcggtgaagatggtgcacgttttggtccatctttaatgcctggtgggtcagcagaagcatggccgcacatcaagaacatctttcaatctattgccgccaaatcaaacggtgagccatgctgcgaatgggtggggcctgccggttctggtcactatgtgaagatggtacacaacggtatcgagtacggtgatatgcagttgatttgcgaggcttacgatatcatgaaacgaattggccggtttacggataaagagatcagtgaagtatttgacaagtggaacactggagttttggattctttcttgattgaaatcacgagggacattttaaaattcgatgacgtcgacggtaagccattggtggaaaaaattatggatactgccggtcaaaagggtactggtaaatggactgcaatcaacgccttggatttaggaatgccagtcactttaattggggaggctgttttcgctcgttgtttgtcagccataaaggacgaacgtaaaagagcttcgaaacttctggcaggaccaacagtaccaaaggatgcaatacatgatagagaacaatttgtgtatgatttggaacaagcattatacgcttcaaagattatttcatatgctcaaggtttcatgctgatccgcgaagctgccagatcatacggctggaaattaaacaacccagctattgctctaatgtggagaggtggctgtataatcagatctgtgttcttagctgagattacgaaggcttatagggacgatccagatttggaaaatttattattcaacgagttcttcgcttctgcagttactaaggcccaatccggttggagaagaactattgcccttgctgctacttacggtattccaactccagctttctctactgctttagcgttttacgacggctatagatctgagaggctaccagcaaacttgttacaagcgcaacgtgattattttggcgctcatacatttagaattttacctgaatgtgcttctgcccatttgccagtagacaaggatattcatatcaattggactgggcacggaggtaatatatcttcctcaacctaccaagcttaa

SEQ ID NO: 414 - protein, 492 aa, Saccharomyces cerevisiae
MSKAVGDLGLVGLAVMGQNLILNAADHGFTVVAYNRTQSKVDRFLANEAKGKSIIGATSIEDLVAKLKKPRKIMLLIKAGAPVDTLIKELVPHLDKGDIIIDGGNSHFPDTNRRYEELTKQGILFVGSGVSGGEDGARFGPSLMPGGSAEAWPHIKNIFQSIAAKSNGEPCCEWVGPAGSGHYVKMVHNGIEYGDMQLICEAYDIMKRIGRFTDKEISEVFDKWNTGVLDSFLIEITRDILKFDDVDGKPLVEKIMDTAGQKGTGKWTAINALDLGMPVTLIGEAVFARCLSAIKDERKRASKLLAGPTVPKDAIHDREQFVYDLEQALYASKIISYAQGFMLIREAARSYGWKLNNPAIALMWRGGCIIRSVFLAEITKAYRDDPDLENLLFNEFFASAVTKAQSGWRRTIALAATYGIPTPAFSTALAFYDGYRSERLPANLLQAQRDYFGAHTFRILPECASAHLPVDKDIHINWTGHGGNISSSTYQA

SEQ ID NO: 415 - DNA, 1470 bp, Saccharomyces cerevisiae
atgtctgctgatttcggtttgattggtttggccgtcatgggtcaaaatttgatcttgaacgctgctgaccacggtttcactgtttgtgcttacaacagaactcaatccaaggtcgaccatttcttggccaatgaagctaagggcaaatctatcatcggtgctacttccattgaagatttcatctccaaattgaagagacctagaaaggtcatgcttttggttaaagctggtgctccagttgacgctttgatcaaccaaatcgtcccacttttggaaaagggtgatattatcatcgatggtggtaactctcacttcccagattctaatagacgttacgaagaattgaagaagaagggtattcttttcgttggttctggtgtctccggtggtgaggaaggtgcccgttacggtccatctttgatgccaggtggttctgaagaagcttggccacatattaagaacatcttccaatccatctctgctaaatccgacggtgaaccatgttgcgaatgggttggcccagccggtgctggtcactacgtcaagatggttcacaacggtattgaatacggtgatatgcaattgatttgtgaagcttatgacatcatgaagagattgggtgggtttaccgataaggaaatcagtgacgtttttgccaaatggaacaatggtgtcttggattccttcttggtcgaaattaccagagatattttgaaattcgacgacgtcgacggtaagccattagttgaaaaaatcatggatactgctggtcaaaagggtactggtaagtggactgccatcaacgccttggatttgggtatgccagttactttgattggtgaagctgtctttgcccgttgtctatctgctttgaagaacgagagaattagagcctccaaggtcttaccaggcccagaagttccaaaagacgccgtcaaggacagagaacaatttgtcgatgatttggaacaagctttgtatgcttccaagattatttcttacgctcaaggtttcatgttgatccgtgaagctgctgctacttatggctggaaactaaacaaccctgccatcgctttgatgtggagaggtggttgtatcattagatctgttttcttgggtcaaatcacaaaggcctacagagaagaaccagatttggaaaacttgttgttcaacaagttcttcgctgatgccgtcaccaaggctcaatctggttggagaaagtcaattgcgttggctaccacctacggtatcccaacaccagccttttccaccgctttgtctttctacgatgggtacagatctgaaagattgccagccaacttactacaagctcaacgtgactactttggtgctcacactttcagagtgttgccagaatgtgcttctgacaacttgccagtagacaaggatatccatatcaactggactggccacggtggtaatgtttcttcctctacataccaagcttaa

SEQ ID NO: 416 - protein, 489 aa, Saccharomyces cerevisiae
MSADFGLIGLAVMGQNLILNAADHGFTVCAYNRTQSKVDHFLANEAKGKSIIGATSIEDFISKLKRPRKVMLLVKAGAPVDALINQIVPLLEKGDIIIDGGNSHFPDSNRRYEELKKKGILFVGSGVSGGEEGARYGPSLMPGGSEEAWPHIKNIFQSISAKSDGEPCCEWVGPAGAGHYVKMVHNGIEYGDMQLICEAYDIMKRLGGFTDKEISDVFAKWNNGVLDSFLVEITRDILKFDDVDGKPLVEKIMDTAGQKGTGKWTAINALDLGMPVTLIGEAVFARCLSALKNERIRASKVLPGPEVPKDAVKDREQFVDDLEQALYASKIISYAQGFMLIREAAATYGWKLNNPAIALMWRGGCIIRSVFLGQITKAYREEPDLENLLFNKFFADAVTKAQSGWRKSIALATTYGIPTPAFSTALSFYDGYRSERLPANLLQAQRDYFGAHTFRVLPECASDNLPVDKDIHINWTGHGGNVSSSTYQA

SEQ ID NO: 417 - DNA, 2046 bp, Saccharomyces cerevisiae
atggcacagttctccgacattgataaacttgcggtttccactttaagattactttccgttgaccaggtggaaagcgcacaatctggccacccaggtgcaccactaggattggcaccagttgcccatgtaattttcaagcaactgcgctgtaaccctaacaatgaacattggatcaatagagacaggtttgttctgtcgaacggtcactcatgcgctcttctgtactcaatgctccatctattaggatacgattactctatcgaggacttgagacaatttagacaagtaaactcaaggacaccgggtcatccagaattccactcagcgggagtggaaatcacttccggtccgctaggccagggtatctcaaatgctgttggtatggcaatagcgcaggccaactttgccgccacttataacgaggatggctttcccatttccgactcatatacgtttgctattgtaggggatggttgcttacaagagggtgtttcttcggagacctcttccttagcgggacatctgcaattgggtaacttgattacgttttatgacagtaatagcatttccattgacggtaaaacctcgtactcgttcgacgaagatgttttgaagcgatacgaggcatatggttgggaagtcatggaagtcgataaaggagacgacgatatggaatccatttctagcgctttggaaaaggcaaaactatcgaaggacaagccaaccataatcaaggtaactactacaattggatttgggtccctacaacagggtactgctggtgttcatgggtccgctttgaaggcagatgatgttaaacagttgaagaagaggtgggggtttgacccaaataaatcatttgtagtacctcaagaggtgtacgattattataagaagactgttgtggaacccggtcaaaaacttaatgaggaatgggataggatgtttgaagaatacaaaaccaaatttcccgagaagggtaaagaattgcaaagaagattgaatggtgagttaccggaaggttgggaaaagcatttaccgaagtttactccggacgacgatgctctggcaacaagaaagacatcccagcaggtgctgacgaacatggtccaagttttgcctgaattgatcggtggttctgccgatttgacaccttcgaatctgacaaggtgggaaggcgcggtagatttccaacctcccattacccaactaggtaactatgcaggaaggtacattagatacggtgtgagggaacacggaatgggtgccattatgaacggtatctctgcctttggtgcaaactacaagccttacggtggtacctttttgaacttcgtctcttatgctgcaggagccgttaggttagccgccttgtctggtaatccagtcatttgggttgcaacacatgactctatcgggcttggtgaggatggtccaacgcaccaacctattgaaactctggctcacttgagggctattccaaacatgcatgtatggagacctgctgatggtaacgaaacttctgctgcgtattattctgctatcaaatctggtcgaacaccatctgttgtggctttatcacgacagaatcttcctcaattggagcattcctcttttgaaaaagccttgaagggtggctatgtgatccatgacgtggagaatcctgatattatcctggtgtcaacaggatcagaagtctccatttctatagatgcagccaaaaaattgtacgatactaaaaaaatcaaagcaagagttgtttccctgccagacttttatacttttgacaggcaaagtgaagaatacagattctctgttctaccagacggtgttccgatcatgtcctttgaagtattggctacttcaagctggggtaagtatgctcatcaatcgttcggactcgacgaatttggtcgttcaggcaaggggcctgaaatttacaaattgttcgatttcacagcggacggtgttgcgtcaagggctgaaaagacaatcaattactacaaaggaaagcagttgctttctcctatgggaagagctttctaa

SEQ ID NO: 418 - protein, 681 aa, Saccharomyces cerevisiae
MAQFSDIDKLAVSTLRLLSVDQVESAQSGHPGAPLGLAPVAHVIFKQLRCNPNNEHWINRDRFVLSNGHSCALLYSMLHLLGYDYSIEDLRQFRQVNSRTPGHPEFHSAGVEITSGPLGQGISNAVGMAIAQANFAATYNEDGFPISDSYTFAIVGDGCLQEGVSSETSSLAGHLQLGNLITFYDSNSISIDGKTSYSFDEDVLKRYEAYGWEVMEVDKGDDDMESISSALEKAKLSKDKPTIIKVTTTIGFGSLQQGTAGVHGSALKADDVKQLKKRWGFDPNKSFVVPQEVYDYYKKTVVEPGQKLNEEWDRMFEEYKTKFPEKGKELQRRLNGELPEGWEKHLPKFTPDDDALATRKTSQQVLTNMVQVLPELIGGSADLTPSNLTRWEGAVDFQPPITQLGNYAGRYIRYGVREHGMGAIMNGISAFGANYKPYGGTFLNFVSYAAGAVRLAALSGNPVIWVATHDSIGLGEDGPTHQPIETLAHLRAIPNMHVWRPADGNETSAAYYSAIKSGRTPSVVALSRQNLPQLEHSSFEKALKGGYVIHDVENPDIILVSTGSEVSISIDAAKKLYDTKKIKARVVSLPDFYTFDRQSEEYRFSVLPDGVPIMSFEVLATSSWGKYAHQSFGLDEFGRSGKGPEIYKLFDFTADGVASRAEKTINYYKGKQLLSPMGRAF

SEQ ID NO: 419 - DNA, 2043 bp, Saccharomyces cerevisiae
atgactcaattcactgacattgataagctagccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaa

SEQ ID NO: 420 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 421 - DNA, 717 bp, Saccharomyces cerevisiae
atggtcaaaccaattatagctcccagtatccttgcttctgacttcgccaacttgggttgcgaatgtcataaggtcatcaacgccggcgcagattggttacatatcgatgtcatggacggccattttgttccaaacattactctgggccaaccaattgttacctccctacgtcgttctgtgccacgccctggcgatgctagcaacacagaaaagaagcccactgcgttcttcgattgtcacatgatggttgaaaatcctgaaaaatgggtcgacgattttgctaaatgtggtgctgaccaatttacgttccactacgaggccacacaagaccctttgcatttagttaagttgattaagtctaagggcatcaaagctgcatgcgccatcaaacctggtacttctgttgacgttttatttgaactagctcctcatttggatatggctcttgttatgactgtggaacctgggtttggaggccaaaaattcatggaagacatgatgccaaaagtggaaactttgagagccaagttcccccatttgaatatccaagtcgatggtggtttgggcaaggagaccatcccgaaagccgccaaagccggtgccaacgttattgtcgctggtaccagtgttttcactgcagctgacccgcacgatgttatctccttcatgaaagaagaagtctcgaaggaattgcgttctagagatttgctagattag

SEQ ID NO: 422 - protein, 238 aa, Saccharomyces cerevisiae
MVKPIIAPSILASDFANLGCECHKVINAGADWLHIDVMDGHFVPNITLGQPIVTSLRRSVPRPGDASNTEKKPTAFFDCHMMVENPEKWVDDFAKCGADQFTFHYEATQDPLHLVKLIKSKGIKAACAIKPGTSVDVLFELAPHLDMALVMTVEPGFGGQKFMEDMMPKVETLRAKFPHLNIQVDGGLGKETIPKAAKAGANVIVAGTSVFTAADPHDVISFMKEEVSKELRSRDLLD

SEQ ID NO: 423 - DNA, 1047 bp, Saccharomyces cerevisiae
atgccaactctagtaaatggaccaagaagagactctaccgaagggtttgataccgatatcatcactcttcctagattcataatcgagcaccagaagcaatttaagaacgctactggtgatttcacattagtactgaatgccttgcaattcgcgttcaaatttgtatctcacaccatcagacgtgctgaattggttaacttggttgggttagcaggcgcttccaacttcactggtgaccagcaaaagaagttggacgttctaggtgatgaaatatttatcaatgccatgagggctagtgggatcatcaaggtccttgtatctgaagaacaggaagacttgatcgtttttcccacaaacacgggctcatacgcagtgtgttgtgatcctattgatggctcctcaaatttggacgccggtgtctccgttggaactatcgcgtctatattcagactgctaccagactcatcaggtactataaacgacgtactgagatgtggtaaagaaatggtagccgcttgctatgccatgtacggatcctctacgcatctagtattgacattgggtgatggagttgatgggtttaccttagacacaaacttgggcgaattcatcttgactcatcctaacttaagaattccgcctcaaaaggccatctactcaattaatgaaggtaacaccctctactggaacgagactataagaacatttattgagaaagtcaaacaaccccaagcagacaacaacaacaagcctttctcggctaggtatgttggatccatggttgctgatgttcacaggacgtttctttacggtggccttttcgcatacccttgcgacaagaagagccccaacggaaaactgaggttgctttatgaggccttcccaatggctttcttaatggaacaagcagggggaaaagcggtcaacgatcgcggagagagaatcttggatttggtgccaagtcatatccatgacaaatcttctatttggttgggttcttcaggtgaaattgacaaatttttagaccatattggcaagtcacagtag

SEQ ID NO: 424 - protein, 348 aa, Saccharomyces cerevisiae
MPTLVNGPRRDSTEGFDTDIITLPRFIIEHQKQFKNATGDFTLVLNALQFAFKFVSHTIRRAELVNLVGLAGASNFTGDQQKKLDVLGDEIFINAMRASGIIKVLVSEEQEDLIVFPTNTGSYAVCCDPIDGSSNLDAGVSVGTIASIFRLLPDSSGTINDVLRCGKEMVAACYAMYGSSTHLVLTLGDGVDGFTLDTNLGEFILTHPNLRIPPQKAIYSINEGNTLYWNETIRTFIEKVKQPQADNNNKPFSARYVGSMVADVHRTFLYGGLFAYPCDKKSPNGKLRLLYEAFPMAFLMEQAGGKAVNDRGERILDLVPSHIHDKSSIWLGSSGEIDKFLDHIGKSQ

SEQ ID NO: 425 - DNA, 2964 bp, Saccharomyces cerevisiae
atgcaatctcaagattcatgctacggtgttgcattcagatctatcatcacaaatgatgaagctttattcaagaagaccattcacttttatcacactctaggatttgcaactgtgaaagatttcaacaaattcaaacatggtgaaaatagcttactatcttcagggacttcccaagattccttgagagaagtttggttagaatctttcaagttgagtgaggttgatgcttctgggttccgtataccacaacaagaagctactaacaaggctcaaagtcaaggtgctctattaaagattcgtttagtgatgtctgctccaatcgatgaaactttcgacaccaacgaaaccgccacaatcacttatttctctactgatttgaacaagattgtcgagaaattcccaaaacaagccgaaaaattgtccgataccttagtgtttttgaaagatccaatgggcaacaacatcaccttctcaggcttagctaatgcaaccgattccgctccaacttccaaagatgctttcttagaagctacctccgaagacgaaatcatctctagagcttcttccgatgcttctgacttactaagacaaacattgggctcttctcaaaagaagaagaagattgctgtcatgacttctggtggtgattctccaggtatgaatgccgctgttcgtgccgttgttcgtacaggtatacatttcggctgtgatgtttttgctgtttacgaaggttacgaaggtttactaagaggcggtaaatatttaaagaaaatggcttgggaagatgtcagaggttggttaagtgaaggtggtactttgattggtactgctcgttctatggaattcagaaagcgtgagggtcgtagacaagctgcaggcaatttaatttcgcaaggtattgacgctttggttgtttgtggtggtgatggttctttaaccggtgctgatcttttcagacacgaatggccatctttggttgatgaattggttgcagaaggtagattcactaaagaagaagtcgccccatacaagaatttgtccattgttggtcttgtcggttccatcgataatgatatgtctggtactgactctaccattggtgcttattctgctttggaaagaatctgtgaaatggttgactacattgatgccaccgctaaatcccactcccgtgcctttgttgttgaagttatgggtagacattgtggttggttggccttgatggctggtattgctaccggtgccgattacatttttattccagaaagagctgttcctcacggaaaatggcaggacgaattgaaggaagtgtgccaaagacacagaagtaagggtagaagaaataacacaattattgtcgctgaaggtgctttagatgatcaattaaaccctgttactgccaatgacgtcaaagatgctttgattgaattgggtctagacaccaaggtaaccattctaggtcacgttcaaagaggtggtacagctgttgctcatgacagatggttagctactctacaaggtgtcgatgctgttaaggccgttctggaatttacccctgaaactccttctccattaattggtattttagaaaacaagataattagaatgccattggttgaatctgtgaagttgactaaatctgttgccactgccattgaaaacaaagatttcgataaggcaatttctttaagagacacagaatttattgaactttacgaaaacttcttatccactaccgttaaagatgatggttccgaattattgccagtatctgacagactaaacattggtattgtccatgttggtgccccatctgctgctttgaacgctgccacccgtgccgcaactctatactgtttgtctcacggccataaaccatacgctatcatgaatggtttcagtggattgattcaaaccggtgaagtgaaggaactatcatggattgatgtcgaaaactggcataacttgggtggttccgaaatcggtacgaacagatctgttgcttcagaagatttgggtaccattgcttactacttccaaaagaacaagctagacggtttgattattcttggtggttttgaaggtttcaggtccttgaagcaattgcgtgacggtagaacccaacacccaatctttaacattccaatgtgtttgattccagccactgtttctaacaacgttccaggtactgaatactcacttggtgttgatacctgtttgaacgcattagtcaattacactgatgacatcaaacagagtgcttctgcgacaagaagaagagtcttcgtctgtgaagtccaaggtggtcactctggttacatcgcttctttcactggtttaatcactggtgctgtttcagtgtacactccagaaaagaagatcgacttagcttctatcagagaagatataactctattaaaagagaactttcgtcacgacaaaggtgaaaacagaaacggtaagctattggttagaaacgaacaagcttctagcgtatatagcactcaattgttggctgacatcatctctgaagcaagcaagggtaagtttggtgttagaactgctatcccaggccatgttcaacaaggtggtgttccatcttctaaagaccgtgtcaccgcttccagatttgctgtcaaatgtatcaagtttatcgaacaatggaacaagaaaaatgaagcttctccaaacactgacgctaaggttttgagattcaagttcgatactcacggtgaaaaggtaccaactgttgagcacgaagatgactctgctgctgttatctgtgttaatggttctcacgtttccttcaagccaattgctaacctttgggaaaacgaaaccaacgttgaattaagaaagggttttgaagttcactgggctgaatacaacaagattggtgacatcctgtccggtagattaaagttgagagctgaggtagccgctttagccgctgaaaacaaatga

SEQ ID NO: 426 - protein, 987 aa, Saccharomyces cerevisiae
MQSQDSCYGVAFRSIITNDEALFKKTIHFYHTLGFATVKDFNKFKHGENSLLSSGTSQDSLREVWLESFKLSEVDASGFRIPQQEATNKAQSQGALLKIRLVMSAPIDETFDTNETATITYFSTDLNKIVEKFPKQAEKLSDTLVFLKDPMGNNITFSGLANATDSAPTSKDAFLEATSEDEIISRASSDASDLLRQTLGSSQKKKKIAVMTSGGDSPGMNAAVRAVVRTGIHFGCDVFAVYEGYEGLLRGGKYLKKMAWEDVRGWLSEGGTLIGTARSMEFRKREGRRQAAGNLISQGIDALVVCGGDGSLTGADLFRHEWPSLVDELVAEGRFTKEEVAPYKNLSIVGLVGSIDNDMSGTDSTIGAYSALERICEMVDYIDATAKSHSRAFVVEVMGRHCGWLALMAGIATGADYIFIPERAVPHGKWQDELKEVCQRHRSKGRRNNTIIVAEGALDDQLNPVTANDVKDALIELGLDTKVTILGHVQRGGTAVAHDRWLATLQGVDAVKAVLEFTPETPSPLIGILENKIIRMPLVESVKLTKSVATAIENKDFDKAISLRDTEFIELYENFLSTTVKDDGSELLPVSDRLNIGIVHVGAPSAALNAATRAATLYCLSHGHKPYAIMNGFSGLIQTGEVKELSWIDVENWHNLGGSEIGTNRSVASEDLGTIAYYFQKNKLDGLIILGGFEGFRSLKQLRDGRTQHPIFNIPMCLIPATVSNNVPGTEYSLGVDTCLNALVNYTDDIKQSASATRRRVFVCEVQGGHSGYIASFTGLITGAVSVYTPEKKIDLASIREDITLLKENFRHDKGENRNGKLLVRNEQASSVYSTQLLADIISEASKGKFGVRTAIPGHVQQGGVPSSKDRVTASRFAVKCIKFIEQWNKKNEASPNTDAKVLRFKFDTHGEKVPTVEHEDDSAAVICVNGSHVSFKPIANLWENETNVELRKGFEVHWAEYNKIGDILSGRLKLRAEVAALAAENK

SEQ ID NO: 427 - DNA, 2880 bp, Saccharomyces cerevisiae
atgactgttactactccttttgtgaatggtacttcttattgtaccgtcactgcatattccgttcaatcttataaagctgccatagatttttacaccaagtttttgtcattagaaaaccgctcttctccagatgaaaactccactttattgtctaacgattccatctctttgaagatccttctacgtcctgatgaaaaaatcaataaaaatgttgaggctcatttgaaggaattgaacagtattaccaagactcaagactggagatcacatgccacccaatccttggtatttaacacttccgacatcttggcagtcaaggacactctaaatgctatgaacgctcctcttcaaggctacccaacagaactatttccaatgcagttgtacactttggacccattaggtaacgttgttggtgttacttctactaagaacgcagtttcaaccaagccaactccaccaccagcaccagaagcttctgctgagtctggtctttcctctaaagttcactcttacactgatttggcttaccgtatgaaaaccaccgacacctatccatctctgccaaagccattgaacaggcctcaaaaggcaattgccgtcatgacttccggtggtgatgctccaggtatgaactctaacgttagagccatcgtgcgttccgctatcttcaaaggttgtcgtgcctttgttgtcatggaaggttatgaaggtttggttcgtggtggtccagaatacatcaaggaattccactgggaagacgtccgtggttggtctgctgaaggtggtaccaacattggtactgcccgttgtatggaattcaagaagcgcgaaggtagattattgggtgcccaacatttgattgaggccggtgtcgatgctttgatcgtttgtggtggtgacggttctttgactggtgctgatctgtttagatcagaatggccttctttgatcgaggaattgttgaaaacaaacagaatttccaacgaacaatacgaaagaatgaagcatttgaatatttgcggtactgtcggttctattgataacgatatgtccaccacggatgctactattggtgcttactctgccttggacagaatctgtaaggccatcgattacgttgaagccactgccaactctcactcaagagctttcgttgttgaagttatgggtagaaactgtggttggttagctttattagctggtatcgccacttccgctgactatatctttattccagagaagccagccacttccagcgaatggcaagatcaaatgtgtgacattgtctccaagcacagatcaaggggtaagagaaccaccattgttgttgttgcagaaggtgctatcgctgctgacttgaccccaatttctccaagcgacgtccacaaagttctagttgacagattaggtttggatacaagaattactaccttaggtcacgttcaaagaggtggtactgctgttgcttacgaccgtatcttggctactttacaaggtcttgaggccgttaatgccgttttggaatccactccagacaccccatcaccattgattgctgttaacgaaaacaaaattgttcgtaaaccattaatggaatccgtcaagttgaccaaagcagttgcagaagccattcaagctaaggatttcaagagagctatgtctttaagagacactgagttcattgaacatttaaacaatttcatggctatcaactctgctgaccacaacgaaccaaagctaccaaaggacaagagactgaagattgccattgttaatgtcggtgctccagctggtggtatcaactctgccgtctactcgatggctacttactgtatgtcccaaggtcacagaccatacgctatctacaatggttggtctggtttggcaagacatgaaagtgttcgttctttgaactggaaggatatgttgggttggcaatcccgtggtggttctgaaatcggtactaacagagtcactccagaagaagcagatctaggtatgattgcttactatttccaaaagtacgaatttgatggtttgatcatcgttggtggtttcgaagcttttgaatctttacatcaattagagagagcaagagaaagttatccagctttcagaatcccaatggtcttgataccagctactttgtctaacaatgttccaggtactgaatactctttgggttctgataccgctttgaatgctctaatggaatactgtgatgttgttaaacaatccgcttcttcaaccagaggtagagccttcgttgtcgattgtcaaggtggtaactcaggctatttggccacttacgcttctttggctgttggtgctcaagtctcttatgtcccagaagaaggtatttctttggagcaattgtccgaggatattgaatacttagctcaatcttttgaaaaggcagaaggtagaggtagatttggtaaattgattttgaagagtacaaacgcttctaaggctttatcagccactaaattggctgaagttattactgctgaagccgatggcagatttgacgctaagccagcttatccaggtcatgtacaacaaggtggtttgccatctccaattgatagaacaagagccactagaatggccattaaagctgtcggcttcatcaaagacaaccaagctgccattgctgaagctcgtgctgccgaagaaaacttcaacgctgatgacaagaccatttctgacactgctgctgtcgttggtgttaagggttcacatgtcgtttacaactccattagacaattgtatgactatgaaactgaagtttccatgagaatgccaaaggtcattcactggcaagctaccagactcattgctgaccatttggttggaagaaagagagttgattaa

SEQ ID NO: 428 - protein, 959 aa, Saccharomyces cerevisiae
MTVTTPFVNGTSYCTVTAYSVQSYKAAIDFYTKFLSLENRSSPDENSTLLSNDSISLKILLRPDEKINKNVEAHLKELNSITKTQDWRSHATQSLVFNTSDILAVKDTLNAMNAPLQGYPTELFPMQLYTLDPLGNVVGVTSTKNAVSTKPTPPPAPEASAESGLSSKVHSYTDLAYRMKTTDTYPSLPKPLNRPQKAIAVMTSGGDAPGMNSNVRAIVRSAIFKGCRAFVVMEGYEGLVRGGPEYIKEFHWEDVRGWSAEGGTNIGTARCMEFKKREGRLLGAQHLIEAGVDALIVCGGDGSLTGADLFRSEWPSLIEELLKTNRISNEQYERMKHLNICGTVGSIDNDMSTTDATIGAYSALDRICKAIDYVEATANSHSRAFVVEVMGRNCGWLALLAGIATSADYIFIPEKPATSSEWQDQMCDIVSKHRSRGKRTTIVVVAEGAIAADLTPISPSDVHKVLVDRLGLDTRITTLGHVQRGGTAVAYDRILATLQGLEAVNAVLESTPDTPSPLIAVNENKIVRKPLMESVKLTKAVAEAIQAKDFKRAMSLRDTEFIEHLNNFMAINSADHNEPKLPKDKRLKIAIVNVGAPAGGINSAVYSMATYCMSQGHRPYAIYNGWSGLARHESVRSLNWKDMLGWQSRGGSEIGTNRVTPEEADLGMIAYYFQKYEFDGLIIVGGFEAFESLHQLERARESYPAFRIPMVLIPATLSNNVPGTEYSLGSDTALNALMEYCDVVKQSASSTRGRAFVVDCQGGNSGYLATYASLAVGAQVSYVPEEGISLEQLSEDIEYLAQSFEKAEGRGRFGKLILKSTNASKALSATKLAEVITAEADGRFDAKPAYPGHVQQGGLPSPIDRTRATRMAIKAVGFIKDNQAAIAEARAAEENFNADDKTISDTAAVVGVKGSHVVYNSIRQLYDYETEVSMRMPKVIHWQATRLIADHLVGRKRVD

SEQ ID NO: 429 - DNA, 777 bp, Saccharomyces cerevisiae
atggctgccggtgtcccaaaaattgatgcgttagaatctttgggcaatcctttggaggatgccaagagagctgcagcatacagagcagttgatgaaaatttaaaatttgatgatcacaaaattattggaattggtagtggtagcacagtggtttatgttgccgaaagaattggacaatatttgcatgaccctaaattttatgaagtagcgtctaaattcatttgcattccaacaggattccaatcaagaaacttgattttggataacaagttgcaattaggctccattgaacagtatcctcgcattgatatagcgtttgacggtgctgatgaagtggatgagaatttacaattaattaaaggtggtggtgcttgtctatttcaagaaaaattggttagtactagtgctaaaaccttcattgtcgttgctgattcaagaaaaaagtcaccaaaacatttaggtaagaactggaggcaaggtgttcccattgaaattgtaccttcctcatacgtgagggtcaagaatgatctattagaacaattgcatgctgaaaaagttgacatcagacaaggaggttctgctaaagcaggtcctgttgtaactgacaataataacttcattatcgatgcggatttcggtgaaatttccgatccaagaaaattgcatagagaaatcaaactgttagtgggcgtggtggaaacaggtttattcatcgacaacgcttcaaaagcctacttcggtaattctgacggtagtgttgaagttaccgaaaagtga

SEQ ID NO: 430 - protein, 258 aa, Saccharomyces cerevisiae
MAAGVPKIDALESLGNPLEDAKRAAAYRAVDENLKFDDHKIIGIGSGSTVVYVAERIGQYLHDPKFYEVASKFICIPTGFQSRNLILDNKLQLGSIEQYPRIDIAFDGADEVDENLQLIKGGGACLFQEKLVSTSAKTFIVVADSRKKSPKHLGKNWRQGVPIEIVPSSYVRVKNDLLEQLHAEKVDIRQGGSAKAGPVVTDNNNFIIDADFGEISDPRKLHREIKLLVGVVETGLFIDNASKAYFGNSDGSVEVTEK

SEQ ID NO: 431 - DNA, 1002 bp, Saccharomyces cerevisiae
atgtcagaaccttcagagaaaaaacaaaaagttgctacctcatctttagaacaattaaaaaaggccgggacccatgtggttgctgattcgggtgattttgaggctatttcgaagtatgaaccacaggattcaacaactaacccttctctgatattggctgcttcaaagttggagaaatatgcgaggtttatcgacgctgccgttgaatatggtagaaagcatggcaaaactgatcatgagaaaatcgagaacgccatggataagattttggtggaattcggcacccagattttgaaggtagtcccaggaagggtttccactgaagtagacgcaaggttatcttttgataagaaggcgacggtgaagaaggctctccacatcatcaaactatataaagatgcgggagtacctaaagaaagagtcttgatcaagatagcttctacgtgggagggtatccaagctgctagagaattggaagtaaagcatggtattcattgtaatatgacattactgttttcctttacgcaagcagtagcctgtgcggaggcaaatgtcacattgatctccccatttgttggaaggattatggacttttacaaggccctttcaggcaaagactatactgcagaaactgatcccggtgttctttctgttaagaagatatacagttactataagaggcatggttatgcaactgaggtaatggcggcttctttcaggaatttagatgaattgaaggcgttagccggtattgataatatgactctcccactgaaccttctagaacagttatatgaatcaacagatcctattgaaaacaaattgaattctgagagtgctaaggaagaaggcgtcgaaaaagtctcttttatcaacgacgagcctcatttcagatatgttttaaacgaagatcaaatggctacagaaaaactatcagatggtatcagaaaattctccgcagacattgaagcactgtacaaactggttgaagaaaaaatgtga

SEQ ID NO: 432 - protein, 333 aa, Saccharomyces cerevisiae
MSEPSEKKQKVATSSLEQLKKAGTHVVADSGDFEAISKYEPQDSTTNPSLILAASKLEKYARFIDAAVEYGRKHGKTDHEKIENAMDKILVEFGTQILKVVPGRVSTEVDARLSFDKKATVKKALHIIKLYKDAGVPKERVLIKIASTWEGIQAARELEVKHGIHCNMTLLFSFTQAVACAEANVTLISPFVGRIMDFYKALSGKDYTAETDPGVLSVKKIYSYYKRHGYATEVMAASFRNLDELKALAGIDNMTLPLNLLEQLYESTDPIENKLNSESAKEEGVEKVSFINDEPHFRYVLNEDQMATEKLSDGIRKFSADIEALYKLVEEKM

SEQ ID NO: 433 - DNA, 1008 bp, Saccharomyces cerevisiae
atgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaa

SEQ ID NO: 434 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 435 - DNA, 1002 bp, Saccharomyces cerevisiae
atgggtattacagtaataggttctctaaactatgatttggacacatttacggatagattacctaacgctggagaaactttcagggctaaccacttcgaaacacatgctggtggtaagggattgaaccaagctgcggccattggtaaattaaaaaaccccagcagcagatatagtgttcgaatgattggtaatgttggaaatgatacatttggtaaacaattgaaggacactttatccgattgcggagtcgatatcactcacgtcggtacttacgaaggcattaatacgggtaccgctaccatactaattgaagagaaagctggtggccaaaataggatattgattgtagaaggtgctaacagcaagactatttatgacccgaaacagttgtgtgaaatttttccagagggcaaggaggaagaagagtatgttgtttttcaacacgaaattcctgatcctctttccattattaaatggatacatgcgaacaggccgaattttcagatcgtatataacccctcacctttcaaggccatgcctaagaaagattgggagttggtagaccttttggtcgttaatgaaattgagggtcttcaaatcgtggaaagtgtatttgataatgaacttgttgaagaaataagggagaagataaaggacgactttttaggagaatatcgtaaaatttgtgagcttttgtatgaaaaactcatgaatcgaaagaaaagaggaattgtggttatgactttgggttcgagaggggtgcttttctgttcgcacgaaagccctgaagtacaattccttccggctattcaaaatgtttcggttgttgatactacaggagctggagatactttcctgggcggtttggttactcaattgtatcaaggagagaccttgtctacggctataaagttctctacattagctagttcattgaccattcaaagaaaaggtgctgctgaaagcatgccactgtataaagatgttcagaaagatgcataa

SEQ ID NO: 436 - protein, 333 aa, Saccharomyces cerevisiae
MGITVIGSLNYDLDTFTDRLPNAGETFRANHFETHAGGKGLNQAAAIGKLKNPSSRYSVRMIGNVGNDTFGKQLKDTLSDCGVDITHVGTYEGINTGTATILIEEKAGGQNRILIVEGANSKTIYDPKQLCEIFPEGKEEEEYVVFQHEIPDPLSIIKWIHANRPNFQIVYNPSPFKAMPKKDWELVDLLVVNEIEGLQIVESVFDNELVEEIREKIKDDFLGEYRKICELLYEKLMNRKKRGIVVMTLGSRGVLFCSHESPEVQFLPAIQNVSVVDTTGAGDTFLGGLVTQLYQGETLSTAIKFSTLASSLTIQRKGAAESMPLYKDVQKDA

SEQ ID NO: 437 - DNA, 1080 bp, Saccharomyces cerevisiae
atgggtgttgaacaaatcttaaagagaaagaccggtgtcatcgttggtgaagatgtccacaacttattcacttacgctaaggaacacaagttcgctattccagctattaacgtcacctcttcttctactgccgtcgctgctttagaagctgctagagacagcaagtccccaatcattttgcaaacctctaacggtggtgctgcttacttcgctggtaagggtatctctaacgaaggtcaaaatgcttccatcaagggtgctattgccgctgcccactacatcagatccattgctccagcttacggtatcccagttgtcttacactctgaccactgtgccaagaagttgttgccatggttcgatggtatgttggaagctgatgaagcttacttcaaggaacacggtgaaccattattctcctcccacatgttggatttgtctgaagaaaccgatgaagaaaacatctctacttgtgtcaagtacttcaagagaatggccgctatggaccaatggttagaaatggaaatcggtattaccggtggtgaagaagatggtgttaacaacgaaaacgctgacaaggaagacttgtacaccaagccagaacaagtttacaacgtctacaaggctttgcacccaatctctccaaacttctccattgctgctgctttcggtaactgtcacggtttgtacgctggtgacatcgctttgagaccagaaatcttggctgaacaccaaaagtacaccagagaacaagttggttgcaaggaagaaaagccattgttcttggtcttccacggtggttccggttctactgtccaagaattccacactggtattgacaacggtgttgtcaaggtcaacttggacactgactgtcaatacgcttacttgactggtatcagagactacgtcttgaacaagaaggactacataatgtccccagtcggtaacccagaaggtccagaaaagccaaacaagaagttcttcgacccaagagtctgggttagagaaggtgaaaagaccatgggtgctaagatcaccaagtctttggaaactttccgtaccactaacactttataa

SEQ ID NO: 438 - protein, 359 aa, Saccharomyces cerevisiae
MGVEQILKRKTGVIVGEDVHNLFTYAKEHKFAIPAINVTSSSTAVAALEAARDSKSPIILQTSNGGAAYFAGKGISNEGQNASIKGAIAAAHYIRSIAPAYGIPVVLHSDHCAKKLLPWFDGMLEADEAYFKEHGEPLFSSHMLDLSEETDEENISTCVKYFKRMAAMDQWLEMEIGITGGEEDGVNNENADKEDLYTKPEQVYNVYKALHPISPNFSIAAAFGNCHGLYAGDIALRPEILAEHQKYTREQVGCKEEKPLFLVFHGGSGSTVQEFHTGIDNGVVKVNLDTDCQYAYLTGIRDYVLNKKDYIMSPVGNPEGPEKPNKKFFDPRVWVREGEKTMGAKITKSLETFRTTNTL

SEQ ID NO: 439 - DNA, 984 bp, Saccharomyces cerevisiae
atgaattcagagtctcgagaagatatggctataaatagtatcaaattgctagcgggaaactcccatcctgatttggctgaacaaatatcgaaaaagttaggtattccactttccaaagttggtgtgtaccagtattctaataaagaaacctctgtcaccataggtgagagccttcgcgacgaagatgtgtatattatccaaactggaataggtgaacaagaaattaatgatttcttgatggaattattaattttaattcatgcttgccaaattgcatctgcaagaaagatcactactgtaatacccaattttccatatgcaagacaagacaagaaagataaatcccgggcgcccattaccgcaaagttggttgccaatttattgcaaactgctggtgctgatcatgtcatcacaatggatctccatgcctcccaaattcaagggtttttccatatcccggttgacaacctatatgcagaaccaagtgttttaaattatattagagcccggaaaacagatttcgacaatgctattttggtgtcgcctgatgcaggtggtgctaagagagtagctgctttggctgacaagttagatttaaattttgctttgattcacaaagagaggcaaaaagctaacgaggtttcaaaaatggtgcttgttggtgatgttaccaataaatcatgtttattagttgatgatatggcggatacttgtggtacgttggtaaaagcttgtgatacgttgatggagcatggtgccaaagaagttatagctattgttacacacggtattttctccggttcagcaagagaaaagctaagaaatagtagattgtctagaattgtttgcacaaataccgttccggtagatttggatttacctattgctgaccagatcgatattagtcccacgttcgctgaagctataagaagactacacaatggtgaatccgtgtcatatttgttcacccatgctccagtatag

SEQ ID NO: 440 - protein, 327 aa, Saccharomyces cerevisiae
MNSESREDMAINSIKLLAGNSHPDLAEQISKKLGIPLSKVGVYQYSNKETSVTIGESLRDEDVYIIQTGIGEQEINDFLMELLILIHACQIASARKITTVIPNFPYARQDKKDKSRAPITAKLVANLLQTAGADHVITMDLHASQIQGFFHIPVDNLYAEPSVLNYIRARKTDFDNAILVSPDAGGAKRVAALADKLDLNFALIHKERQKANEVSKMVLVGDVTNKSCLLVDDMADTCGTLVKACDTLMEHGAKEVIAIVTHGIFSGSAREKLRNSRLSRIVCTNTVPVDLDLPIADQIDISPTFAEAIRRLHNGESVSYLFTHAPV

SEQ ID NO: 441 - DNA, 957 bp, Saccharomyces cerevisiae
atgtctacaaacagtattaagttgttagctggtaactcacatcctgggcttgctgagcttatttcccaaaggcttggtgtaccgttatctaaagtgggggtatatcagtattcaaataaagagacgtctgttacaataggggaaagcattcgtgatgaagacgtatatatcatccaaactggttacggggaacatgaaatcaacgatttcttaatggaattacttatcttaattcacgcttgtaagacggcatcagtcagaagaattacggcagtaataccaaatttcccttatgctagacaagataagaaggataaatcgcgggcgcccatcactgcgaagctgattgccaatcttttggaaactgcgggttgtgatcatgttattactatggatctccatgcttcacaaattcaagggttttttcatatcccggttgataatttatacggtgaaccaagtgtgttgaattacataaggaccaaaacagatttcaataatgcgatactggtatcgcctgatgctggtggtgcgaaaagggtggcttctttagcggacaagctggatatgaattttgccttgattcataaggagaggcaaaaagctaatgaagtttcgcgaatgctgcttgtcggtgatgttgctggtaagtcatgcttgttgattgacgatatggctgacacgtgtggtacgctggtgaaagcctgtgacacattaatggatcacggtgctaaggaggttatcgctattgtcacacatggtatattttctggctcagctagggagaagttaataaatagtaggttatcaagaattgtttgcaccaacacggtacctgttgatttggatttggacattgtcgatcaggtggatatcagccctacgattgcagaagctatcagaagattgcataatggagaatcagtctcatatttatttacacatgcgcctgtttaa

SEQ ID NO: 442 - protein, 318 aa, Saccharomyces cerevisiae
MSTNSIKLLAGNSHPGLAELISQRLGVPLSKVGVYQYSNKETSVTIGESIRDEDVYIIQTGYGEHEINDFLMELLILIHACKTASVRRITAVIPNFPYARQDKKDKSRAPITAKLIANLLETAGCDHVITMDLHASQIQGFFHIPVDNLYGEPSVLNYIRTKTDFNNAILVSPDAGGAKRVASLADKLDMNFALIHKERQKANEVSRMLLVGDVAGKSCLLIDDMADTCGTLVKACDTLMDHGAKEVIAIVTHGIFSGSAREKLINSRLSRIVCTNTVPVDLDLDIVDQVDISPTIAEAIRRLHNGESVSYLFTHAPV

SEQ ID NO: 443 - DNA, 963 bp, Saccharomyces cerevisiae
atgccaacaaattccatcaaattattagcaccagatgtccaccgaggtttagctgaacttgttgccaaaaggctaggtctacagttaacaagcagcaaactaaagagagatccaaccggtgaggtttccttttctattggagaatctgttagggaccaagatatctttatcatcacgcaaattggctctggtgtcgtgaacgatcgtgttctagaactactgatcatgatcaatgcttcgaagactgcgtctgcaagaagaatcactgctattattccaaatttcccatatgcaagacaagatagaaaggataagtctcgtgctcctattaccgctaaattgatggccgatatgttaacaacagccgggtgtgatcatgttattactatggatttgcatgcttcccaaattcaagggttcttcgacgtcccagtagataatttatatgcagaaccaagcgtagttaggtatatcaaggaaaatgtcaactatatggattcaataataatttcgccagatgctggtggggcaaaacgtgctgccactttggctgatcgtttggatctaaattttgcattgatccataaggaaagagctcgtgccaatgaggtttctcgtatggttcttgttggggatgttactgataagatttgtatcattgttgatgatatggccgatacttgtggtactttagctaaggctgctgaaattcttttggagaatagagctaaatctgttattgctattgtcacgcacggtgttttatcaggcagagctattgaaaatattaacaattctaaactggatagagttgtttgcacaaatactgttccatttgaagaaaaaatcaagaagtgcccaaaactggcagtcattgacatcagtagtgtgctggctgaaagtattcgtcgtttgcataatggtgaaagtatttcttatttatttaagaattatcccttataa

SEQ ID NO: 444 - protein, 320 aa, Saccharomyces cerevisiae
MPTNSIKLLAPDVHRGLAELVAKRLGLQLTSSKLKRDPTGEVSFSIGESVRDQDIFIITQIGSGVVNDRVLELLIMINASKTASARRITAIIPNFPYARQDRKDKSRAPITAKLMADMLTTAGCDHVITMDLHASQIQGFFDVPVDNLYAEPSVVRYIKENVNYMDSIIISPDAGGAKRAATLADRLDLNFALIHKERARANEVSRMVLVGDVTDKICIIVDDMADTCGTLAKAAEILLENRAKSVIAIVTHGVLSGRAIENINNSKLDRVVCTNTVPFEEKIKKCPKLAVIDISSVLAESIRRLHNGESISYLFKNYPL

SEQ ID NO: 445 - DNA, 1284 bp, Saccharomyces cerevisiae
atgcgtaagtgtaaaatttttgttggtaactctcacccagagttgggtaatatggtttgccaaagattaggtatcgaaccagctccatgtacattaaaaaaatttgcaaatggtgaaacgtcagttcaaatcggtgtatctgttcgtgacgaagatgtgtatgtcattcaatctggttctccatccataaatgatgacattatggaactgttgattttagtttctgcctgtaggggcggatctgctaggaaaattacagcagtcattcctcaatttccttactctaaacaatgtaaaatgaagagacacagaggcgctattactgcaaggatgttggccaatctgttggtcatggctggcgctgatcatgttgtgtctatggaccttcatgcttctcaaatgcaggggtttttcaccaaaccagtcgataacctatacgggggccctagtttagcgaaatggattagagaaaatgttgaagactatgaagatgcagtagttgtctctaagaatccaggtggtactaaaagagttacagccctggcggattctttgaagatcaactttgctatgatccatactgatcgtcgtcgttctaaggatctgtattctcaaaacaaggatttgcaacaactaaagttgagaaagcaatcaatgttgaggaaaaatagaccgatcatcagacaaggtgaccatccaaatgaggaggaaaacattattttaagtaacggtattcaaactgctaggatccgtaacggccacgttatcggtgacgatgaggcagacgatgatgaagacgctatcttagaatctgacagcgaattgcattctattgatggcttagactcgcatggcttgggcggtacatatgatgctgtcgactccgaagacgaagaggaaatcccagttctctatagggaacaactgattacattggtcggaaacgtccgtggtcgctctgccatcattctagatgatatgattgatagacctgggtcatttattagcgctgctgaacatttggtgcaaaattgtggtgccaaaaaggtttatgttgttgctactcacggtatattcactggagactgtttggaagaacttgaaaagtccgatgccattgacacaattgtggtcacaaatacatatccgatttctggcgaacgcatagctgggtccaagaaattggttactatcgatgtttctccaattttcgcagaatgtatccgtcgtgatcattatggtgagagtatttccgttttatttgactctttggcagctctgtaa

SEQ ID NO: 446 - protein, 427 aa, Saccharomyces cerevisiae
MRKCKIFVGNSHPELGNMVCQRLGIEPAPCTLKKFANGETSVQIGVSVRDEDVYVIQSGSPSINDDIMELLILVSACRGGSARKITAVIPQFPYSKQCKMKRHRGAITARMLANLLVMAGADHVVSMDLHASQMQGFFTKPVDNLYGGPSLAKWIRENVEDYEDAVVVSKNPGGTKRVTALADSLKINFAMIHTDRRRSKDLYSQNKDLQQLKLRKQSMLRKNRPIIRQGDHPNEEENIILSNGIQTARIRNGHVIGDDEADDDEDAILESDSELHSIDGLDSHGLGGTYDAVDSEDEEEIPVLYREQLITLVGNVRGRSAIILDDMIDRPGSFISAAEHLVQNCGAKKVYVVATHGIFTGDCLEELEKSDAIDTIVVTNTYPISGERIAGSKKLVTIDVSPIFAECIRRDHYGESISVLFDSLAAL

SEQ ID NO: 447 - DNA, 1491 bp, Saccharomyces cerevisiae
atgtcaatgagtaatattgttgtttttggaggggactcgcaccccgagttagttactaagatctgtgaaaatttggacattcacccatcgaaagtagaattagggaagttttctaatggggaaacgaacattgctcttcgcgaatctgttcgtgaaaaggatgtatatatcatccagagtggttgtggccaggtgaacgacacgttcatgcagttgctgattttaatcagtgcctgcaagtccgcttctgcctcgagggttacagccgtaatgccatatctctgctactcgagacagccagatattccatatactgccaagggtgctcccataatttccaagcctaaagaaaactatacttttgaatcgcatccaggcacacccgtgtcatcttctttaatgacgcaaagaccaggtgctgagagctcgttgaagagtttggatagtgcaatacgatcaactatcaacttagaaaatcctcaacctatcagaacaccaaacagcagtgctacggcgaataacaatttcgacatcaagaagacgctttctttttcaagaattcctatgattcccggtggtaagttacaaaatacaagcaatagcacggacgctggtgaattgttcaacgctcaaaatgcaggctataagctatgggtagtacaagccggtactttgattgctcatttgttgagtgctgcaggtgctgaccatgtgatcacaatggatttgcacgatccacagttccctgggttttttgacattccagtggataatctctactgtaaacccattgcacaaaactacatccagcatcgcattccagattatcaggatgctgtgatcgtttctccagatgctggtggtgcaaagagagctacggctattgcagacgccttggaattgtccttcgccctaattcataaagaaagaagatctcagttattgaagggccctccagatgcgacgttaacctctggtggttcgttaccagtatctccaaggccattagttactactttggtttcctcccaaaatactacttcttcaggtgccactggggttgcggcccttgaaatgaagaaaacaacttcaacatcttccacctcgtcgcaatcttctaattcgtccaagttcgttcaaactaccatgcttgttggcgatgttagaaacaaggtgtgtattatagtcgacgacttggtggatacttcatacactattacaagagctgcgaaattgttgaaggatcaaggatctaccaaagtttatgccttaataacgcacggtgttttttccggtgatgcgctagaaagaatcggccaaagtagtatagataagttgatcatttctaacacggttcctcaagatagaacactacagtacctaggtaaggacagagtggatgttattgatgtctcctgcataatcggtgaagcaattagaagaatccataacggtgaatccatttctatgttgttcgagcatggatggtag

SEQ ID NO: 448 - protein, 496 aa, Saccharomyces cerevisiae
MSMSNIVVFGGDSHPELVTKICENLDIHPSKVELGKFSNGETNIALRESVREKDVYIIQSGCGQVNDTFMQLLILISACKSASASRVTAVMPYLCYSRQPDIPYTAKGAPIISKPKENYTFESHPGTPVSSSLMTQRPGAESSLKSLDSAIRSTINLENPQPIRTPNSSATANNNFDIKKTLSFSRIPMIPGGKLQNTSNSTDAGELFNAQNAGYKLWVVQAGTLIAHLLSAAGADHVITMDLHDPQFPGFFDIPVDNLYCKPIAQNYIQHRIPDYQDAVIVSPDAGGAKRATAIADALELSFALIHKERRSQLLKGPPDATLTSGGSLPVSPRPLVTTLVSSQNTTSSGATGVAALEMKKTTSTSSTSSQSSNSSKFVQTTMLVGDVRNKVCIIVDDLVDTSYTITRAAKLLKDQGSTKVYALITHGVFSGDALERIGQSSIDKLIISNTVPQDRTLQYLGKDRVDVIDVSCIIGEAIRRIHNGESISMLFEHGW

SEQ ID NO: 449 - DNA, 1713 bp, Saccharomyces cerevisiae
atgtcacttctaatagattctgtaccaacagttgcttataaggaccaaaaaccgggtacttcaggtttacgtaagaagaccaaggttttcatggatgagcctcattatactgagaacttcattcaagcaacaatgcaatctatccctaatggctcagagggaaccactttagttgttggaggagatggtcgtttctacaacgatgttatcatgaacaagattgccgcagtaggtgctgcaaacggtgtcagaaagttagtcattggtcaaggcggtttactttcaacaccagctgcttctcatataattagaacatacgaggaaaagtgtaccggtggtggtatcatattaactgcctcacacaacccaggcggtccagagaatgatttaggtatcaagtataatttacctaatggtgggccagctccagagagtgtcactaacgctatctgggaagcgtctaaaaaattaactcactataaaattataaagaacttccccaagttgaatttgaacaagcttggtaaaaaccaaaaatatggcccattgttagtggacataattgatcctgccaaagcatacgttcaatttctgaaggaaatttttgattttgacttaattaaaagcttcttagcgaaacagcgcaaagacaaagggtggaagttgttgtttgactccttaaatggtattacaggaccatatggtaaggctatatttgttgatgaatttggtttaccggcagaggaagttcttcaaaattggcaccctttacctgatttcggcggtttacatcccgatccgaatctaacctatgcacgaactcttgttgacagggttgaccgcgaaaaaattgcctttggagcagcctccgatggtgatggtgataggaatatgatttacggttatggccctgctttcgtttcgccaggtgattctgttgccattattgccgaatatgcacccgaaattccatacttcgccaaacaaggtatttatggcttggcacgttcatttcctacatcctcagccattgatcgtgttgcagcaaaaaagggattaagatgttacgaagttccaaccggctggaaattcttctgtgccttatttgatgctaaaaagctatcaatctgtggtgaagaatccttcggtacaggttccaatcatatcagagaaaaggacggtctatgggccattattgcttggttaaatatcttggctatctaccataggcgtaaccctgaaaaggaagcttcgatcaaaactattcaggacgaattttggaacgagtatggccgtactttcttcacaagatacgattacgaacatatcgaatgcgagcaggccgaaaaagttgtagctcttttgagtgaatttgtatcaaggccaaacgtttgtggctcccacttcccagctgatgagtctttaaccgttatcgattgtggtgatttttcgtatagagatctagatggctccatctctgaaaatcaaggccttttcgtaaagttttcgaatgggactaaatttgttttgaggttatccggcacaggcagttctggtgcaacaataagattatacgtagaaaagtatactgataaaaaggagaactatggccaaacagctgacgtcttcttgaaacccgtcatcaactccattgtaaaattcttaagatttaaagaaattttaggaacagacgaaccaacagtccgcacatag

SEQ ID NO: 450 - protein, 570 aa, Saccharomyces cerevisiae
MSLLIDSVPTVAYKDQKPGTSGLRKKTKVFMDEPHYTENFIQATMQSIPNGSEGTTLVVGGDGRFYNDVIMNKIAAVGAANGVRKLVIGQGGLLSTPAASHIIRTYEEKCTGGGIILTASHNPGGPENDLGIKYNLPNGGPAPESVTNAIWEASKKLTHYKIIKNFPKLNLNKLGKNQKYGPLLVDIIDPAKAYVQFLKEIFDFDLIKSFLAKQRKDKGWKLLFDSLNGITGPYGKAIFVDEFGLPAEEVLQNWHPLPDFGGLHPDPNLTYARTLVDRVDREKIAFGAASDGDGDRNMIYGYGPAFVSPGDSVAIIAEYAPEIPYFAKQGIYGLARSFPTSSAIDRVAAKKGLRCYEVPTGWKFFCALFDAKKLSICGEESFGTGSNHIREKDGLWAIIAWLNILAIYHRRNPEKEASIKTIQDEFWNEYGRTFFTRYDYEHIECEQAEKVVALLSEFVSRPNVCGSHFPADESLTVIDCGDFSYRDLDGSISENQGLFVKFSNGTKFVLRLSGTGSSGATIRLYVEKYTDKKENYGQTADVFLKPVINSIVKFLRFKEILGTDEPTVRT

SEQ ID NO: 451 - DNA, 1710 bp, Saccharomyces cerevisiae
atgtcatttcaaattgaaacggttcccaccaaaccatatgaagaccaaaagcctggtacctctggtttgcgtaagaagacaaaggtgtttaaagacgaacctaactacacagaaaatttcattcaatcgatcatggaagctattccagagggttctaaaggtgccactcttgttgtcggtggtgatgggcgttactacaatgatgtcattcttcataagattgccgctatcggtgctgccaacggtattaaaaagttagttattggccagcatggtcttctgtctacgccagccgcttctcacatcatgagaacctacgaggaaaaatgtactggtggtattatcttaaccgcctcacataatccaggtggtccagaaaatgacatgggtattaagtataacttatccaatgggggtcctgctcctgaatccgtcacaaatgctatttgggagatttccaaaaagcttaccagctataagattatcaaagacttcccagaactagacttgggtacgataggcaagaacaagaaatacggtccattactcgttgacattatcgatattacaaaagattatgtcaacttcttgaaggaaatcttcgatttcgacttaatcaagaaattcatcgataatcaacgttctactaagaattggaagttactgtttgacagtatgaacggtgtaactggaccatacggtaaggctattttcgttgatgaatttggtttaccggcggatgaggttttacaaaactggcatccttctccggattttggtggtatgcatccagatccaaacttaacttatgccagttcgttagtgaaaagagtagatcgtgaaaagattgagtttggtgctgcatccgatggtgatggtgatagaaatatgatttacggttacggcccatctttcgtttctccaggtgactccgtcgcaattattgccgaatatgcagctgaaatcccatatttcgccaagcaaggtatatatggtctggcccgttcattccctacctcaggagccatagaccgtgttgccaaggcccatggtctaaactgttatgaggtcccaactggctggaaatttttctgtgctttgttcgacgctaaaaaattatctatttgtggtgaagaatcgtttggtactggttccaaccacgtaagggaaaaggacggtgtttgggccattatggcgtggttgaacatcttggccatttacaacaagcatcatccggagaacgaagcttctattaagacgatacagaatgaattctgggcaaagtacggccgtactttcttcactcgttatgattttgaaaaagttgaaacagaaaaagctaacaagattgtcgatcaattgagagcatatgttaccaaatcgggtgttgttaattccgccttcccagccgatgagtctcttaaggtcaccgattgtggtgatttttcatacacagatttggacggttctgtttctgaccatcaaggtttatatgtcaagctttccaatggtgcaagattcgttctaagattgtcaggtacaggttcttcaggtgctaccattagattgtacattgaaaaatactgcgatgataaatcacaataccaaaagacagctgaagaatacttgaagccaattattaactcggtcatcaagttcttgaactttaaacaagttttaggaactgaagaaccaacggttcgtacttaa

SEQ ID NO: 452 - protein, 569 aa, Saccharomyces cerevisiae
MSFQIETVPTKPYEDQKPGTSGLRKKTKVFKDEPNYTENFIQSIMEAIPEGSKGATLVVGGDGRYYNDVILHKIAAIGAANGIKKLVIGQHGLLSTPAASHIMRTYEEKCTGGIILTASHNPGGPENDMGIKYNLSNGGPAPESVTNAIWEISKKLTSYKIIKDFPELDLGTIGKNKKYGPLLVDIIDITKDYVNFLKEIFDFDLIKKFIDNQRSTKNWKLLFDSMNGVTGPYGKAIFVDEFGLPADEVLQNWHPSPDFGGMHPDPNLTYASSLVKRVDREKIEFGAASDGDGDRNMIYGYGPSFVSPGDSVAIIAEYAAEIPYFAKQGIYGLARSFPTSGAIDRVAKAHGLNCYEVPTGWKFFCALFDAKKLSICGEESFGTGSNHVREKDGVWAIMAWLNILAIYNKHHPENEASIKTIQNEFWAKYGRTFFTRYDFEKVETEKANKIVDQLRAYVTKSGVVNSAFPADESLKVTDCGDFSYTDLDGSVSDHQGLYVKLSNGARFVLRLSGTGSSGATIRLYIEKYCDDKSQYQKTAEEYLKPIINSVIKFLNFKQVLGTEEPTVRT

SEQ ID NO: 453 - DNA, 1869 bp, Saccharomyces cerevisiae
atgttgcaaggaattttagaaaccgtaccatctgacttgaaagatccgatatcattatggtttaagcaagaccgcaacccaaaaactatagaagaggtcaccgctctctgcaaaaaatccgactggaatgagttacacaaaagatttgattctagaattcagtttggcactgctggtttaagatcgcaaatgcaagctggctttagcaggatgaatactttagtagtcatacaagcgtctcagggattggcaacttatgtaagacaacagtttccagacaatttggtagctgttgtgggacacgatcatagattccattctaaggagttcgctagagctactgctgctgcatttcttttaaaaggatttaaggtacattatttgaatcctgaccacgaatttgttcatacccctttagttccctttgcagtggataagctaaaggcctccgttggcgtaatgataacagcaagtcacaacccaaaaatggataatggatataaagtatactattccaatggatgccaaatcattccacctcacgatcatgccatctctgattccattgacgcaaatttagaaccatgggccaatgtgtgggatttcgacgatgttctaaataaggctctcaaacaagggaaattgatgtattccagagaagaaatgctgaagttatatttagaggaggtttctaaaaatctggtagaaatcaacccattaaagcttgaagtaaaagccaaaccttggttcgtttacactccaatgcatggggttggatttgacattttcagcaccatcgtaaaaaaaacactgtgcctggtagaaggtaaggattacctatgtgttcctgaacaacaaaatccagatccttctttcccaactgttggatttcctaaccctgaagaaaaaggtgctttagacattggtataaacttggctgaaaaacatgacattgacttacttgttgccaacgaccctgacgctgatagattctctgttgctgttaaagatatgcagtcaggcgaatggcgacaactaacaggtaacgaaatcggttttctttttgcattttatgaatatcagaaatataaaagtatggacaaagaatttcagcacgttcatccgttggctatgttaaattcaacagtgtcttcacaaatgataaaaaaaatggcagaaatagaagggttccattatgaggatacattaacaggatttaagtggatcggaaatcgtgccatactcttggaaaagaaaggctattacgttccttttggattcgaggaagcaataggctacatgtttccagcaatggagcatgataaggatggtatcagtgcatccattgtcttcttgcaagcctactgtaagtggaaaatagaccacaatttggacccgctaaatgtcttagaaaatggcttcaaaaaatatggcgtgttcaaagagtacaatggctattatgtcgttccaaatccaactgttacaaaagatatatttgactacatcaggaatgtctacactcctgagggcgcgtcatatccttcatctattggtgaagaaatcgaagtactttactatcgagatttaaccactggttaccaatcggataccataaatcataaacctactctacccgtcgatcctacatcacaaatgataacagtatctgctagaccaagtaacggtagtgagaatgagcatatccgcttcactattcgcgggtccggaacagaaccaaaacttaaagtatatattgaagcttgcgcaaatgaagaacaaagagcctctttcttggcgaaattgacttggaatgtgctgagacgtgaatggtttagaccagatgaaatgaatatagttacaaaattttga

SEQ ID NO: 454 - protein, 622 aa, Saccharomyces cerevisiae
MLQGILETVPSDLKDPISLWFKQDRNPKTIEEVTALCKKSDWNELHKRFDSRIQFGTAGLRSQMQAGFSRMNTLVVIQASQGLATYVRQQFPDNLVAVVGHDHRFHSKEFARATAAAFLLKGFKVHYLNPDHEFVHTPLVPFAVDKLKASVGVMITASHNPKMDNGYKVYYSNGCQIIPPHDHAISDSIDANLEPWANVWDFDDVLNKALKQGKLMYSREEMLKLYLEEVSKNLVEINPLKLEVKAKPWFVYTPMHGVGFDIFSTIVKKTLCLVEGKDYLCVPEQQNPDPSFPTVGFPNPEEKGALDIGINLAEKHDIDLLVANDPDADRFSVAVKDMQSGEWRQLTGNEIGFLFAFYEYQKYKSMDKEFQHVHPLAMLNSTVSSQMIKKMAEIEGFHYEDTLTGFKWIGNRAILLEKKGYYVPFGFEEAIGYMFPAMEHDKDGISASIVFLQAYCKWKIDHNLDPLNVLENGFKKYGVFKEYNGYYVVPNPTVTKDIFDYIRNVYTPEGASYPSSIGEEIEVLYYRDLTTGYQSDTINHKPTLPVDPTSQMITVSARPSNGSENEHIRFTIRGSGTEPKLKVYIEACANEEQRASFLAKLTWNVLRREWFRPDEMNIVTKF

SEQ ID NO: 455 - DNA, 951 bp, Escherichia coli
atgagtataaaagagcaaacgttaatgacgccttacctacagtttgaccgcaaccagtgggcagctctgcgtgattccgtacctatgacgttatcggaagatgagatcgcccgtctcaaaggtattaatgaagatctctcgttagaagaagttgccgagatctatttacctttgtcacgtttgctgaacttctatataagctcgaatctgcgccgtcaggcagttctggaacagtttcttggtaccaacgggcaacgcattccttacattatcagtattgctggcagtgtcgcggtggggaaaagtacaaccgcccgtgtattgcaggcgctattaagccgttggccggaacatcgtcgtgttgaactgatcactacagatggcttccttcaccctaatcaggttctgaaagaacgtggtctgatgaagaagaaaggcttcccggaatcgtatgatatgcatcgcctggtgaagtttgtttccgatctcaaatccggcgtgccaaacgttacagcacctgtttactcacatcttatttatgatgtgatcccggatggagataaaacggttgttcagcctgatattttaattcttgaagggttaaatgtcttacagagcgggatggattatccacacgatccacatcatgtatttgtttctgattttgtcgatttttcgatatatgttgatgcaccggaagacttacttcagacatggtatatcaaccgttttctgaaattccgcgaaggggcttttaccgacccggattcctattttcataactacgcgaaattaactaaagaagaagcgattaagactgccatgacattgtggaaagagatcaactggctgaacttaaagcaaaatattctacctactcgtgagcgcgccagtttaatcctgacgaaaagtgctaatcatgcggtagaagaggtcagactacgcaaataa

SEQ ID NO: 456 - protein, 316 aa, Escherichia coli
MSIKEQTLMTPYLQFDRNQWAALRDSVPMTLSEDEIARLKGINEDLSLEEVAEIYLPLSRLLNFYISSNLRRQAVLEQFLGTNGQRIPYIISIAGSVAVGKSTTARVLQALLSRWPEHRRVELITTDGFLHPNQVLKERGLMKKKGFPESYDMHRLVKFVSDLKSGVPNVTAPVYSHLIYDVIPDGDKTVVQPDILILEGLNVLQSGMDYPHDPHHVFVSDFVDFSIYVDAPEDLLQTWYINRFLKFREGAFTDPDSYFHNYAKLTKEEAIKTAMTLWKEINWLNLKQNILPTRERASLILTKSANHAVEEVRLRK

SEQ ID NO: 457 - DNA, 777 bp, Bacillus spp.
ttgttactggttatcgatgtggggaacaccaatactgtacttggtgtatatcatgatggaaaattagaatatcactggcgtatagaaacaagcaggcataaaacagaagatgagtttgggatgattttgcgctccttatttgatcactccgggcttatgtttgaacagatagatggcattattatttcgtcagtagtgccgccaatcatgtttgcgttagaaagaatgtgcacaaaatactttcatatcgagcctcaaattgttggtccaggtatgaaaaccggtttaaatataaaatatgacaatccgaaagaagtaggggcagacagaatcgtaaatgctgtcgctgcgatacacttgtacggcaatccattaattgttgtcgatttcggaaccgccacaacgtactgctatattgatgaaaacaaacaatacatgggcggggcgattgcccctgggattacaatttcgacagaggcgctttactcgcgtgcagcaaagcttcctcgtatcgaaatcacccggcccgacaatattatcggaaaaaacactgttagcgcgatgcaatctggaattttatttggctatgtcggccaagtggaaggaatcgttaagcgaatgaaatggcaggcaaaacaggaaccaaaggtcattgcgacaggaggcctggcgccgctcattgcgaacgaatcagattgtatagacatcgttgatccattcttaaccctaaaagggctggaattgatttatgaaagaaaccgcgtaggaagtgtatag

SEQ ID NO: 458 - protein, 258 aa, Bacillus spp.
MLLVIDVGNTNTVLGVYHDGKLEYHWRIETSRHKTEDEFGMILRSLFDHSGLMFEQIDGIIISSVVPPIMFALERMCTKYFHIEPQIVGPGMKTGLNIKYDNPKEVGADRIVNAVAAIHLYGNPLIVVDFGTATTYCYIDENKQYMGGAIAPGITISTEALYSRAAKLPRIEITRPDNIIGKNTVSAMQSGILFGYVGQVEGIVKRMKWQAKQEPKVIATGGLAPLIANESDCIDIVDPFLTLKGLELIYERNRVGSV

SEQ ID NO: 459 - DNA, 1221 bp, Escherichia coli
atgagcctggccggtaaaaaaatcgttctcggcgttagcggcggtattgctgcctataaaacccctgaactggtgcgtcgtttgcgcgatcgcggggccgacgtccgcgtagccatgaccgaagcggcaaaagcctttatcaccccacttagcttgcaggcggtttctggttatcccgtttccgacagtctgctggacccggcagccgaagccgctatgggccatattgagctgggtaaatgggctgatttagtgattctcgcccctgccacggcagatttgattgcccgtgttgctgccggaatggcgaatgacctggtatcgacgatttgtctggctacacctgcgcctgtagccgtgctccccgccatgaaccagcagatgtaccgtgccgctgccacgcagcataatttagaggtgcttgcttcccgtggtttgctcatctgggggccagacagtggcagtcaggcttgtggtgatatcggtcctgggcgaatgctcgatccgttaaccattgtggatatggcggtagcgcatttttcgcccgtcaacgacctgaaacatctgaacattatgattaccgccggcccgacgcgtgaaccgctcgatccggtgcgttatatctctaatcacagctccggcaagatgggttttgctatcgccgccgccgctgcccgtcgtggcgcgaacgtcacgctggtatcaggtccggtttcactaccgacgccaccgtttgttaaacgtgttgatgtgatgaccgcgctggaaatggaagccgccgtgaatgcttctgtacagcagcaaaatatttttatcggctgcgccgccgtggcggattatcgcgcagctaccgtggccccagagaaaatcaaaaagcaggccacgcagggtgatgaattaacaataaaaatggttaaaaaccccgatatcgtcgcaggcgttgccgcactaaaagaccatcgaccctacgtcgttggatttgccgccgaaacaaataatgtggaagaatacgcccggcaaaaacgtatccgtaaaaaccttgatctgatctgcgcgaacgatgtttcccagccaactcaaggatttaacagcgacaacaacgcattacaccttttctggcaggacggagataaagtcttaccgcttgagcgcaaagagctccttggccaattattactcgacgagatcgtgacccgttatgatgaaaaaaatcgacgttaa

SEQ ID NO: 460 - protein, 406 aa, Escherichia coli
MSLAGKKIVLGVSGGIAAYKTPELVRRLRDRGADVRVAMTEAAKAFITPLSLQAVSGYPVSDSLLDPAAEAAMGHIELGKWADLVILAPATADLIARVAAGMANDLVSTICLATPAPVAVLPAMNQQMYRAAATQHNLEVLASRGLLIWGPDSGSQACGDIGPGRMLDPLTIVDMAVAHFSPVNDLKHLNIMITAGPTREPLDPVRYISNHSSGKMGFAIAAAAARRGANVTLVSGPVSLPTPPFVKRVDVMTALEMEAAVNASVQQQNIFIGCAAVADYRAATVAPEKIKKQATQGDELTIKMVKNPDIVAGVAALKDHRPYVVGFAAETNNVEEYARQKRIRKNLDLICANDVSQPTQGFNSDNNALHLFWQDGDKVLPLERKELLGQLLLDEIVTRYDEKNRR

SEQ ID NO: 461 - DNA, 480 bp, Escherichia coli
atgcaaaaacgggcgatttatccgggtactttcgatcccattaccaatggtcatatcgatatcgtgacgcgcgccacgcagatgttcgatcacgttattctggcgattgccgccagccccagtaaaaaaccgatgtttaccctggaagagcgtgtggcactggcacagcaggcaaccgcgcatctggggaacgtggaagtggtcgggtttagtgatttaatggcgaacttcgcccgtaatcaacacgctacggtgctgattcgtggcctgcgtgcggtggcagattttgaatatgaaatgcagctggcgcatatgaatcgccacttaatgccggaactggaaagtgtgtttctgatgccgtcgaaagagtggtcgtttatctcttcatcgttggtgaaagaggtggcgcgccatcagggcgatgtcacccatttcctgccggagaatgtccatcaggcgctgatggcgaagttagcgtag

SEQ ID NO: 462 - protein, 159 aa, Escherichia coli
MQKRAIYPGTFDPITNGHIDIVTRATQMFDHVILAIAASPSKKPMFTLEERVALAQQATAHLGNVEVVGFSDLMANFARNQHATVLIRGLRAVADFEYEMQLAHMNRHLMPELESVFLMPSKEWSFISSSLVKEVARHQGDVTHFLPENVHQALMAKLA

SEQ ID NO: 463 - DNA, 621 bp, Escherichia coli
atgaggtatatagttgccttaacgggaggcattggcagtggcaagagtaccgttgccaatgcgtttgctgatctcggaattaacgtcattgatgccgatattattgcgcgtcaggtggttgaaccaggtgcacctgcgctacatgccattgctgatcactttggcgctaacatgattgctgctgatggaacattgcagcgccgggccttgcgcgagcggatcttcgccaacccggaagagaaaaactggcttaacgccctgctgcatccgctgattcagcaagagacgcaacaccagatccagcaagctacttccccctatgtactgtgggttgtgccattgctggtagaaaactcactgtataaaaaagcgaatcgagtgcttgtggtggatgtcagcccagaaacgcaacttaagcgcaccatgcagcgcgatgatgtaactcgcgagcatgtcgaacaaatccttgctgctcaggcaacgcgcgaagcccgccttgccgtggcagatgacgtcattgataataacggcgcaccggatgctatcgcatcggatgttgcccgcctgcacgcacactatttgcagcttgcgtcgcagtttgtctcacaggaaaaaccgtaa

SEQ ID NO: 464 - DNA, 747 bp, Escherichia coli; identical to SEQ ID NO: 37

SEQ ID NO: 465 - protein, 248 aa, Escherichia coli; identical to SEQ ID NO: 38

SEQ ID NO: 466 - DNA, 336 bp, Escherichia coli; identical to SEQ ID NO: 39

SEQ ID NO: 467 - protein, 111 aa, Escherichia coli; identical to SEQ ID NO: 40

SEQ ID NO: 468 - DNA, 531 bp, Escherichia coli; identical to SEQ ID NO: 41

SEQ ID NO: 469 - protein, 176 aa, Escherichia coli; identical to SEQ ID NO: 42

SEQ ID NO: 470 - DNA, 522 bp, Escherichia coli; identical to SEQ ID NO: 43

SEQ ID NO: 471 - protein, 173 aa, Escherichia coli; identical to SEQ ID NO: 44

SEQ ID NO: 472 - DNA, 3525 bp, Escherichia coli; identical to SEQ ID NO: 35

SEQ ID NO: 473 - protein, 1174 aa, Escherichia coli; identical to SEQ ID NO: 36

SEQ ID NO: 474 - DNA, 1707 bp, Escherichia coli
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgccgctgaagcgatttacaccccggaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggcacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcattcgcttccccagcgtccatctgaaagactatctgacccgtttggctcagaaagtttccaagaaaaccggtgcattggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacattggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 475 - protein, 568 aa, Escherichia coli
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 476 - DNA, 1404 bp, Escherichia coli
atgaatcaacaggatattgaacaggtggtgaaagcggtactgctgaaaatgcaaagcagtgacacgccgtccgccgccgttcatgagatgggcgttttcgcgtccctggatgacgccgttgcggcagccaaagtcgcccagcaagggttaaaaagcgtggcaatgcgccagttagccattgctgccattcgtgaagcaggcgaaaaacacgccagagatttagcggaacttgccgtcagtgaaaccggcatggggcgcgttgaagataaatttgcaaaaaacgtcgctcaggcgcgcggcacaccaggcgttgagtgcctctctccgcaagtgctgactggcgacaacggcctgaccctaattgaaaacgcaccctggggcgtggtggcttcggtgacgccttccactaacccggcggcaaccgtaattaacaacgccatcagcctgattgccgcgggcaacagcgtcatttttgccccgcatccggcggcgaaaaaagtctcccagcgggcgattacgctgctcaaccaggcgattgttgccgcaggtgggccggaaaacttactggttactgtggcaaatccggatatcgaaaccgcgcaacgcttgttcaagtttccgggtatcggcctgctggtggtaaccggcggcgaagcggtagtagaagcggcgcgtaaacacaccaataaacgtctgattgccgcaggcgctggcaacccgccggtagtggtggatgaaaccgccgacctcgcccgtgccgctcagtccatcgtcaaaggcgcttctttcgataacaacatcatttgtgccgacgaaaaggtactgattgttgttgatagcgtagccgatgaactgatgcgtctgatggaaggccagcacgcggtgaaactgaccgcagaacaggcgcagcagctgcaaccggtgttgctgaaaaatatcgacgagcgcggaaaaggcaccgtcagccgtgactgggttggtcgcgacgcaggcaaaatcgcggcggcaatcggccttaaagttccgcaagaaacgcgcctgctgtttgtggaaaccaccgcagaacatccgtttgccgtgactgaactgatgatgccggtgttgcccgtcgtgcgcgtcgccaacgtggcggatgccattgcgctagcggtgaaactggaaggcggttgccaccacacggcggcaatgcactcgcgcaacatcgaaaacatgaaccagatggcgaatgctattgataccagcattttcgttaagaacggaccgtgcattgccgggctggggctgggcggggaaggctggaccaccatgaccatcaccacgccaaccggtgaaggggtaaccagcgcgcgtacgtttgtccgtctgcgtcgctgtgtattagtcgatgcgtttcgcattgtttaa

SEQ ID NO: 477 - protein, 467 aa, Escherichia coli
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 478 - DNA, 405 bp, Aeromonas cavaie
atgagcgcgcagagcctggaagtgggtcaaaaagctcgcctgtccaaacgttttggtgcagcagaagttgcggccttcgcagcactgtctgaagacttcaatccgctgcatctggacccggcattcgcggcaaccaccgcatttgaacgcccgatcgttcatggtatgctgttggcaagcctgtttagcggtctgctgggtcagcagctgccgggcaaaggtagcatttacctgggtcagagcctgagctttaaactgccggtgttcgtcggtgacgaggtcacggcggaggtcgaggttacggccctgcgtgaggacaagccgatcgcgaccctgaccacccgtattttcacccagggtggcgcgttggccgtgacgggtgaggccgtggtcaaactgccgtaa

SEQ ID NO: 479 - protein, 134 aa, Aeromonas cavaie
MSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLP

(19) United States
(12) Patent Application Publication
(10) Pub. No.: US 2014/0004597 A1
(43) Pub. Date: Jan. 2, 2014

(54) SYNTHETIC PATHWAYS FOR BIOFUEL SYNTHESIS

(71) Applicant: The Regents of the University of California, Oakland, CA (US)

(72) Inventors: Michelle C.Y. CHANG, Berkeley, CA (US); Brooks Bond-Watts, Oakland, CA (US); Miao Wen, Milpitas, CA (US); Jeffrey A. Hanson, Oakland, CA (US)

(73) Assignee: The Regents of the University of California, Oakland, CA (US)

(21) Appl. No.: 13/708,824

(22) Filed: Dec. 7, 2012

Related U.S. Application Data
(63) Continuation of application No. PCT/US2011/040102, filed on Jun. 10, 2011.
(60) Provisional application No. 61/354,129, filed on Jun. 11, 2010.

(51) Int. Cl. C12P 7/16

(52) U.S. Cl. CPC C12P 7/16
USPC 435/252.33; 435/254.21

(57) ABSTRACT

The present disclosure provides optimized recombinant cells for the production of n-butanol. Methods for the use of these cells are also provided. Specifically, the utility of acylating aldehyde dehydrogenases and pyruvate:flavodoxin/ferredoxin-oxidoreductase for the improvement of n-butanol yields from recombinant cells is disclosed.

### CROSS-REFERENCE TO RELATED APPLICATIONS

This application is a Continuation application of PCT/US2011/040102, filed Jun. 10, 2011, which claims the benefit of U.S. Provisional Patent Application Ser. No. 61/354,129 filed Jun. 11, 2010, the contents of which are hereby incorporated by reference in their entirety.

### FIELD

The present disclosure relates to recombinant cells containing improved pathways for biofuel synthesis. In particular, recombinant cells and methods for the synthesis of n-butanol are provided.

### BACKGROUND

Liquid fuels derived from plant biomass are renewable energy sources and the global demand for such biofuels is rising. Ethanol is the most widely used biofuel today, but its low energy return, high vaporizability and miscibility with water present major technical challenges. Alternative biofuels, such as n-butanol, more closely resemble gasoline and have the potential to replace ethanol as the predominant biofuel in the future.

While several microorganisms can produce ethanol as a fermentation product, only few natural micoorganisms can produce n-butanol. Natural n-butanol producers, such as Clostridium acetobutylicum (C. acetobutylicum), can be used for industrial applications but are not as genetically tractable or robust fermentation hosts as, for example, Escherichia coli (E. coli) or Saccharomyces cerevisiae (S. cerevisiae). It is therefore attractive to engineer a recombinant pathway for biofuel production in such host as E. coli or S. cerevisiae.

n-Butanol biosynthesis typically includes several enzymatic steps, whereby different n-butanol synthesizing organisms can utilize different classes and combinations of enzymes to mediate the conversion from pyruvate to n-butanol. Generally, the startpoint of n-butanol synthesis, pyruvate, can be derived through the metabolism of various sugar substrates, including glucose and xylose, but also starches and lignocellulosics. Pyruvate is then converted to acetyl-CoA. Acetyl-CoA is subsequently converted to acetoacetyl-CoA, which is itself converted to 3-hydroxybutyryl-CoA. 3-Hydroxybutyryl-CoA is converted to crotonyl-CoA. Crotonyl-CoA is converted to butyryl-CoA. Finally, butyryl-CoA is converted to n-butanol.

The n-butanol biosynthesis pathway of C. acetobutylicum converting acetyl-CoA to n-butanol can be lifted out and inserted into E. coli, thereby generating a recombinant cell that produces n-butanol (Inui, et al., 2008, Appl. Microbiol. Biotechnol. 77, 1305-16; Atsumi et al., 2008, Metab. Eng. 10, 305-11; Nielsen et al., 2009, Metab. Eng. 11, 262-73). However, the C. acetobutylicum derived n-butanol biosynthesis pathway contains multiple bottlenecks that limit the yields of biofuel production.

In view of these facts and the growing global demand in biofuels, a significant need exists for more productive recombinant cells and improved methods for biofuel synthesis. Specifically, new recombinant cells are needed providing for robust and high-yielding n-butanol synthesis pathways.

### BRIEF SUMMARY

Provided herein are recombinant cells for the production of n-butanol. Also provided are methods for producing n-butanol using the recombinant cells described herein.

Particularly, recombinant cells are provided including recombinant sequences encoding enzymes that constitute a synthetic pathway for n-butanol production. In one embodiment of the invention the enzymes include an acylating aldehyde dehydrogenase catalyzing the conversion of acetaldehyde to acetyl-CoA. In another embodiment the enzymes include a pyruvate:flavodoxin/ferredoxin-oxidoreductase catalyzing the conversion of pyruvate to acetyl-CoA. The acylating aldehyde dehydrogenase or pyruvate:flavodoxin/ferredoxin-oxidoreductase are combined with a keto-thiolase or acetyl-CoA acetyltransferase catalyzing the conversion of acetyl-CoA to acetoacetyl-CoA, an acetoacetyl-CoA reductase or hydroxybutyryl-CoA dehydrogenase catalyzing the conversion of acetoacetyl-CoA to 3-hydroxybutyryl-CoA, a crotonase catalyzing the conversion of 3-hydroxybutyryl-CoA to crotonyl-CoA a crotonyl-CoA reductase, butyryl-CoA dehydrogenase or trans-enoyl-CoA reductase catalyzing the conversion of crotonyl-CoA to butyryl-CoA, and a butyraldehyde/butanol dehydrogenase catalyzing the conversion of butyryl-CoA to n-butanol.

Furthermore, methods for n-butanol production are provided. The methods include the step of growing a recombinant cell of the invention in the presence of a suitable carbon source.

### BRIEF DESCRIPTION OF THE DRAWINGS

FIG. 1 shows the biosynthesis pathways for n-butanol. Enzyme 1 is a pyruvate dehydrogenase or a pyruvate dehydrogenase bypass consisting of a pyruvate decarboxyase and a acylating aldehyde dehydrogenase, or a pyruvate dehydrogenase bypass consisting of a pyruvate decarboxyase, a non-acylating aldehyde dehydrogenase, and an acetyl-CoA synthetase, or a pyruvate:flavodoxin/ferredoxin-oxidoreductase, or a pyruvate formate lyase and formate dehydrogenase; Enzyme 2 is a keto-thiolase or acetyl-CoA acetyltransferase; Enzyme 3 is an acetoacetyl-CoA reductase or hydroxybutyryl-CoA dehydrogenase; Enzyme 4 a crotonase; Enzyme 5 is a trans-2-enoyl-CoA reductase; Enzyme 6 is a butyraldehyde/butanol dehydrogenase. Subclasses of Enzymes 3 and 4, such Enzyme 3.1 and 3.2, may feature different stereoselectivities and produce different chiral intermediates. For example, the acetoacetyl-CoA reductase Hbd produces (S)-hydroxybutyryl-CoA while PhaB produces (R)-hydroxybutyryl-CoA.

FIG. 2 shows the production of n-butanol using different strains (1-6), promoters (6-12), enoyl-CoA reductase and ketoreductase/enoyl-CoA hydratase selection (10, 13-18) and overexpression of a pyruvate dehydrogenase (PDH) (19-20).

FIGS. 3A and 3B show the promoter optimization for Ccr expression and the S-tag analysis of Ccr solubility. FIG. 3A shows butanol production using pBT33-phaA.phaB-crt in combination with ccr-adhE2 and using promoters with variable strengths to transcribe the ccr-adhE2 operon. The plasmids used for expression of the ccr-adhE2 operon were pBAD33-ccr.adhE2, pTrc99a-ccr.adhE2, pCWOri-ccr.adhE2, and pET29a-ccr.adhE2. FIG. 3B shows the relationship between n-butanol production (quantified by GC-MS) and soluble Ccr-Stag protein (quantified by S-Tag Rapid Assay Kit, Novagen).

FIGS. 4A and 4B show the trapping of pathway intermediates by Ter. FIG. 4A shows the reaction catalyzed by Crt is reversible in cell lysate after 2 hours. FIG. 4B shows that Ter is effectively irreversible in cell lysate with no observable reaction occurring within 2 hours.

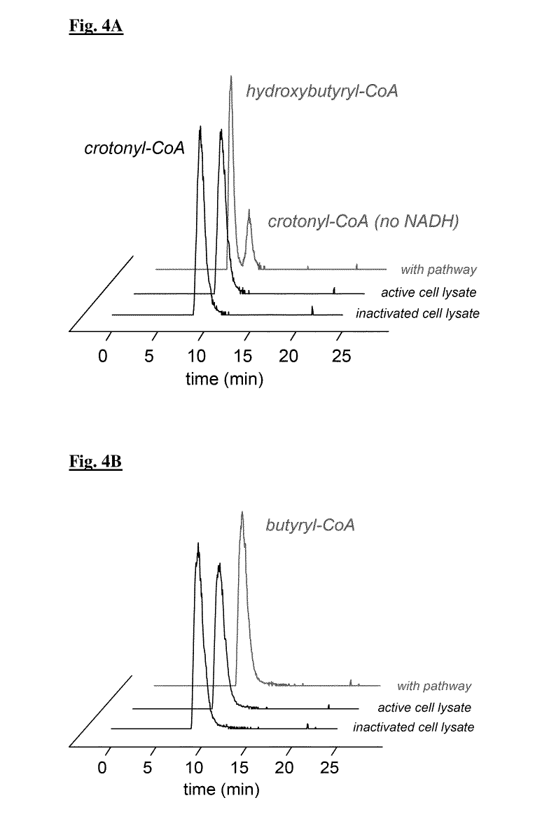

FIG. 5 shows a Neighbor Net graph of Ter from T. denticola (Tucci and Martin, 2007, FEBS Lett. 581 (2007) 1561-66). The scale bar at the lower right indicates estimated substitutions per site. Abbreviations are as follows: β and γ, proteobacteria; bactero, bacteroides; entero, enterobacteria; spiro, spirochete.

FIG. 6 shows the impact of replacing Ccr for Ter on n-butanol yields in recombinant E. coli. Elevating E. coli PDH levels in the presence of Ter results in further increases in n-butanol yields.

FIG. 7 shows n-butanol production in E. coli cell genetically modified to express the butanol biosynthetic pathway of FIG. 1. The product retention time was compared to an authentic n-butanol standard in a chromatograph (left), and a product mass spectrum was compared to an authentic n-butanol standard (right) to confirm the identity of the fermentation product.

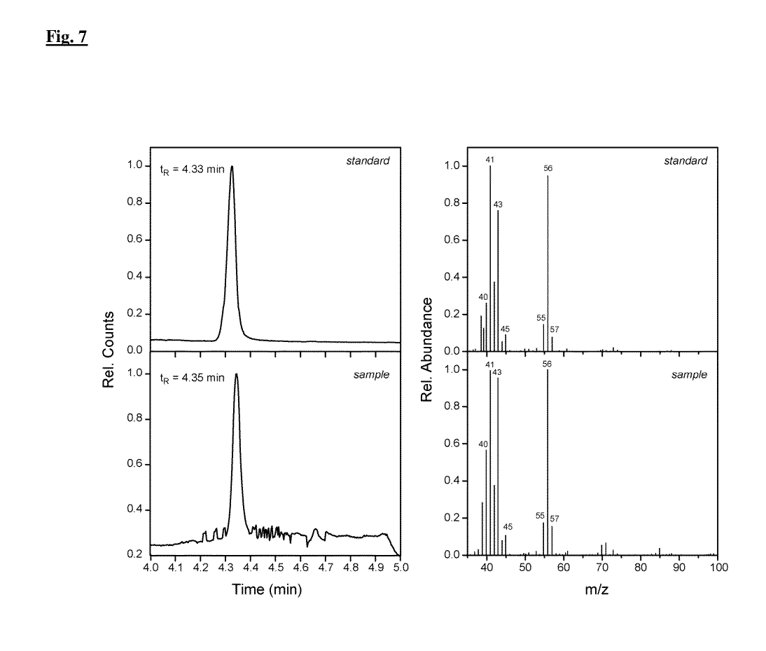

FIG. 8 compares the n-butanol production and the ethanol to butanol ratio in E. coli in the presence of basal levels of acetyl-CoA versus and after overexpression of the variants of E. coli PDH (pyruvate dehydrogenase complex), PFOR complex (pyruvate:flavodoxin/ferredoxin-oxidoreductase (YdbK), a flavodoxin-NADP reductase (Fpr), a ferredoxin (Fdx), and one of two flavodoxins (FldA or FldB) all of which are from E. coli), pyruvate formate lyase (Pfl) and formate dehydrogenase (Fdh), and PDH bypass (a pyruvate decarboxylase from Z. mobilis, an acylating aldehyde dehydrogenase from E. coli, and a pantothenate kinase from E. coli).

FIG. 9 shows total fuel (butanol and ethanol) titer in E. coli DH1 and a knockout strain in the presence of basal levels of acetyl-CoA versus expression of the PDHc bypass (a pyruvate decarboxylase from Z. mobilis, an acetylating aldehyde dehydrogenase from E. coli, and a pantetheinate kinase from E. coli).

FIG. 10 shows the four general pathways for the conversion of pyruvate to acetyl-CoA consisting of a pyruvate dehydrogenase complex, or a pyruvate:flavodoxin/ferrodoxin-oxidoreductase, or a pyruvate dehydrogenase bypass consisting of a pyruvate decarboxyase and acylating aldehyde dehydrogenase, or a pyruvate dehydrogenase bypass consisting of a pyruvate decarboxyase and a non-acylating aldehyde dehydrogenase and an acetyl-CoA synthetase, or pyruvate formate lyase and formate dehydrogenase.

FIG. 11 shows native fermentation pathways in E. coli that compete with fuel production under anaerobic and microaerobic conditions.

FIGS. 12A and 12B show n-butanol production in S. cerevisiae. FIG. 12A shows the recombinant pathway for n-butanol production in recombinant S. cerevisiae cells. FIG. 12B shows butanol production in S. cerevisiae BY4741Δadh. Column 1 shows background level production of butanol. Column 2 shows butanol titer by Saccharomyces cerevisiae BY4741Δadh strain harboring butanol production pathway and PDH bypass.

FIG. 13 shows the pentose phosphate pathway. The pentose phosphate pathway takes in C5 sugars, including xylose and arabinose, and using NADP⁺/H as a cofactor converts the sugars into molecules that can enter into glycolysis and then into the n-butanol producing pathway.

### DETAILED DESCRIPTION OF PREFERRED EMBODIMENTS

The present disclosure relates to recombinant cells producing n-butanol and to methods of using these recombinant cells for the production of n-butanol from fermentable carbon sources.

n-Butanol Synthesis Pathway

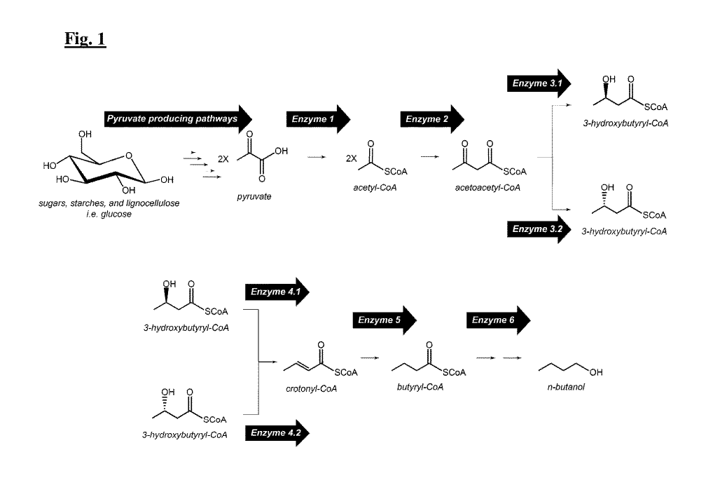

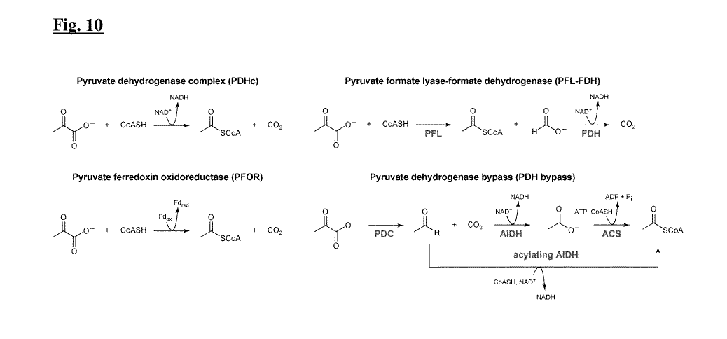

n-Butanol can be produced by a recombinant cell containing recombinant sequences of at least six enzymes catalyzing the generation of acetyl-CoA and its stepwise conversion to n-butanol (FIGS. 1 and 10). Acetyl-CoA can be generated from the glycolysis product pyruvate by means of a pyruvate dehydrogenase complex (PDHc), a pyruvate formate oxidoreductase (PFOR), the combined activities of a pyruvate formate lyase and a formate dehydrogenase (PFL-FDH), or a pyruvate dehydrogenase bypass pathway (PDH bypass). PDH bypass pathways can include a pyruvate dehydrogenase (PDC) in combination with an acylating aldehyde dehydrogenase (AlDH) or a non-acylating aldehyde dehydrogenase and an acetyl-CoA synthetase. The conversion of acetyl-CoA to n-butanol may proceed through the intermediates acetoacetyl-CoA, 3-hydroxybutyryl-CoA, crotonyl-CoA, and butyryl-CoA. The recombinant cells of this invention are engineered to contain efficient heterologous pathways for n-butanol production.

In one embodiment of the invention the recombinant cell contains recombinant sequences encoding i) an acylating aldehyde dehydrogenase catalyzing the conversion of acetaldehyde to acetyl-CoA (FIG. 1, Enzyme 1), ii) a keto-thiolase or acetyl-CoA acetyltransferase catalyzing the conversion of acetyl-CoA to acetoacetyl-CoA (FIG. 1, Enzyme 2), iii) an acetoacetyl-CoA reductase or hydroxybutyryl-CoA dehydrogenase catalyzing the conversion of acetoacetyl-CoA to 3-hydroxybutyryl-CoA (FIG. 1, Enzyme 3), iv) a crotonase catalyzing the conversion of 3-hydroxybutyryl-CoA to crotonyl-CoA (FIG. 1, Enzyme 4), v) a crotonyl-CoA reductase, butyryl-CoA dehydrogenase or trans-enoyl-CoA reductase catalyzing the conversion of crotonyl-CoA to butyryl-CoA (FIG. 1, Enzyme 5), and vi) a butyraldehyde/butanol dehydrogenase catalyzing the conversion of butyryl-CoA to n-butanol (FIG. 1, Enzyme 6).

In one specific embodiment the sequences encoding the acylating aldehyde dehydrogenase, the keto-thiolase or acetyl-CoA acetyltransferase, the acetoacetyl-CoA reductase or hydroxybutyryl-CoA dehydrogenase, the crotonase, the crotonyl-CoA reductase, butyryl-CoA dehydrogenase or trans-enoyl-CoA reductase, and the butyraldehyde/butanol dehydrogenase are linked. In another specific embodiment the sequences are not linked.

Some organisms may not express an endogenous pyruvate decarboxylase or may express only low levels of pyruvate decarboxylase activity that limit the availability of acetaldehyde, the activity of the acylating aldehyde dehydrogenase, and the overall n-butanol yields of the recombinant biosynthesis pathway. Therefore, in some embodiments the recombinant cell further contains a recombinant sequence encoding a pyruvate decarboxylase catalyzing the conversion of pyruvate to acetaldehyde. In another specific embodiment the pyruvate decarboxylase is derived from Z. mobilis or S. cerevisiae.

In one embodiment of the invention the recombinant cell contains recombinant sequences encoding i) a pyruvate:flavodoxin/ferredoxin-oxidoreductase catalyzing the conversion of pyruvate to acetyl-CoA (FIG. 1, Enzyme 1), ii) a keto-thiolase or acetyl-CoA acetyltransferase catalyzing the conversion of acetyl-CoA to acetoacetyl-CoA (FIG. 1, Enzyme 2), iii) an acetoacetyl-CoA reductase or hydroxybutyryl-CoA dehydrogenase catalyzing the conversion of acetoacetyl-CoA to 3-hydroxybutyryl-CoA (FIG. 1, Enzyme 3), iv) a crotonase catalyzing the conversion of 3-hydroxybutyryl-CoA to crotonyl-CoA (FIG. 1, Enzyme 4), v) a crotonyl-CoA reductase, butyryl-CoA dehydrogenase or trans-enoyl-CoA reductase catalyzing the conversion of crotonyl-CoA to butyryl-CoA (FIG. 1, Enzyme 5), and vi) a butyraldehyde/butanol dehydrogenase catalyzing the conversion of butyryl-CoA to n-butanol (FIG. 1, Enzyme 6).

In one specific embodiment the sequences encoding the pyruvate:flavodoxin/ferredoxin-oxidoreductase, the keto-thiolase or acetyl-CoA acetyltransferase, the acetoacetyl-CoA reductase or hydroxybutyryl-CoA dehydrogenase, the crotonase, the crotonyl-CoA reductase, butyryl-CoA dehydrogenase or trans-enoyl-CoA reductase, and the butyraldehyde/butanol dehydrogenase are linked. In another specific embodiment the sequences are not linked.

In one specific embodiment the recombinant cell further comprising recombinant sequences encoding the ferredoxin-NADP reductase from E. coli, the ferredoxin FdC from E. coli, and the flavodoxins FldA and FldB from E. coli.

In one embodiment of the invention the recombinant cell produces n-butanol under aerobic conditions. In one embodiment of the invention the recombinant cell produces n-butanol under microaerobic conditions. Microaerobic conditions refer to an environment where the concentration of oxygen is less than that in the air. In one embodiment of the invention the recombinant cell produces n-butanol under anaerobic conditions. In one specific embodiment the recombinant cell produces more n-butanol under anaerobic conditions than under aerobic or microaerobic conditions. In another specific embodiment the recombinant cell produces near quantitative yields of n-butanol under anaerobic conditions.

In one embodiment of the invention the recombinant cell produces n-butanol and ethanol under aerobic conditions. In one embodiment of the invention the recombinant cell produces n-butanol and ethanol under microaerobic conditions. In one embodiment of the invention the recombinant cell produces n-butanol and ethanol under anaerobic conditions. In one specific embodiment the recombinant cell produces more total levels of n-butanol and ethanol under anaerobic conditions than under aerobic or microaerobic conditions. In another specific embodiment the recombinant cell produces near quantitative yields of n-butanol and ethanol under anaerobic conditions.

In one embodiment of the invention the recombinant cell produces elevated levels of n-butanol compared to a wild-type cell under aerobic conditions. Elevated levels of n-butanol produced by the recombinant cell under aerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 1,000,000-fold compared to the n-butanol levels produced by a wild-type cell under aerobic conditions. In specific embodiments the recombinant cell produces at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L n-butanol under aerobic conditions.

In one embodiment of the invention the recombinant cell produces elevated total levels of n-butanol and ethanol compared to a wild-type cell under aerobic conditions. Elevated total levels of n-butanol and ethanol produced by the recombinant cell under aerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 1,000,000-fold compared to the total levels of n-butanol and ethanol produced by a wild-type cell under aerobic conditions. In specific embodiments the recombinant cell produces under aerobic conditions total levels of n-butanol and ethanol of at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L.

In one embodiment of the invention the recombinant cell produces elevated levels of n-butanol compared to a wild-type cell under anaerobic conditions. Elevated levels of n-butanol produced by the recombinant cell under anaerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 1,000,000-fold compared to the n-butanol levels produced by a wild-type cell under anaerobic conditions. In specific embodiments the recombinant cell produces at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L n-butanol under anaerobic conditions.

In one embodiment of the invention the recombinant cell produces elevated total levels of n-butanol and ethanol compared to a wild-type cell under anaerobic conditions. Elevated total levels of n-butanol and ethanol produced by the recombinant cell under anaerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 1,000,000-fold compared to the total levels of n-butanol and ethanol produced by a wild-type cell under anaerobic conditions. In specific embodiments the recombinant cell produces under anaerobic conditions total levels of n-butanol and ethanol of at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L.

### Enzyme 1: Acetyl-CoA Generation

Recombinant cells of this invention contain at least one recombinant pathway for the production of acetyl-CoA (FIG. 10). In one embodiment of the invention the recombinant cell contains recombinant sequences encoding a pyruvate dehydrogenase complex (PDH). In a specific embodiment the PDH is Pdh from E. coli. In another embodiment the recombinant cell contains recombinant sequences encoding a pyruvate formate lyase (PFL) and a formate dehydrogenase (FDH).

In another embodiment the recombinant cell contains recombinant sequences encoding a pyruvate formate oxidoreductase complex (PFOR). In one specific embodiment PFOR includes a pyruvate:flavodoxin/ferredoxin-oxidoreductase, a flavodoxin-NADP reductase, a ferredoxin, and at least one flavodoxins. In another specific embodiment the recombinant sequences encoding PFOR includes YdbK (SEQ ID NOs: 472, 473), Fpr (SEQ ID NOs: 464, 465), Fdx (SEQ ID NOs: 466, 467), and FldA (SEQ ID NOs: 468, 469), or FldB (SEQ ID NOs: 470, 471) from E. coli.

In another embodiment the recombinant cell contains recombinant sequences encoding a pyruvate dehydrogenase bypass (PDH bypass). In one specific embodiment the PDHc bypass includes recombinant sequences encoding a pyruvate decarboxylase (PDC). In another specific embodiment the PDHc bypass includes recombinant sequences encoding a non-acylating aldehyde dehydrogenase (AlDH). In another specific embodiment the PDH bypass includes recombinant sequences encoding an acetyl-CoA synthetase (ACS). In another specific embodiment the PDHc bypass includes recombinant sequences encoding a PDC, a non-acylating AlDH, and an ACS. In another specific embodiment the PDHc bypass includes recombinant sequences encoding an acetylating AlDH. In a preferred embodiment the PDHc bypass includes recombinant sequences encoding a PDC and an acylating AlDH. In another preferred embodiment the PDHc bypass includes recombinant sequences encoding a PDC from Z. mobitilis and an acylating aldehyde dehydrogenase from E. coli. In another preferred embodiment the PDHc bypass contains recombinant sequences encoding Pdc from Z. mobitilis and EutEA from E. coli.

Recombinant sequences encoding PDHc, PFOR, PFL, FDH, acylating AlDH and non-acylating AlDH enzymes may be derived from all prokaryotic organisms, including proteobacterial, archaebacterial, bacteroidal, enterobacterial, spirochetal organisms, and all eukaryotic organisms, including mammalian, insect, fungal and yeast organisms. Preferred examples include, but are not limited to: E. coli Pdh, which is composed of the three genes aceE (SEQ ID NOs: 1, 2), aceF (SEQ ID NOs: 3, 4), and lpdA (SEQ ID NOs: 5, 6), the E. faecalis Pdh, which is composed of the four genes pdhA (SEQ ID NOs: 7, 8), pdhB (SEQ ID NOs: 9, 10), aceF (SEQ ID NOs: 11, 12), and lpdA (SEQ ID NOs: 13, 14), the E. coli Pfor genes ydbK (SEQ ID NOs: 35, 36), fpr (SEQ ID NOs: 37, 38), fdx (SEQ ID NOs: 39, 40), fldA (SEQ ID NOs: 41, 42), and fldB (SEQ ID NOs: 43, 44), the Z. mobiilis pdc gene (SEQ ID NOs: 474, 475), and the E. coli acetylating aldehyde dehydrogenase gene eutE (SEQ ID NOs: 476, 477).

### Enzyme 2: Keto-Thiolase or Acetyl-CoA Acetyltransferase

Recombinant sequences encoding the keto-thiolase or acetyl-CoA acetyltransferase may be derived from all prokaryotic organisms, including proteobacterial, archaebacterial, bacteroidal, enterobacterial, spirochetal organisms, and all eukaryotic organisms, including mammalian, insect, fungal and yeast organisms. Preferred examples include, but are not limited to: the Rastonia eutrophus acetoacetyl-CoA thiolase/synthase phaA (SEQ ID NOs: 15, 16) and related enzymes from cells that make polyhydroxyalkanoates, C. acetobutylicum acetoacetyl-CoA thiolase/synthase thI, and E. coli acetoacetyl-CoA thiolase/synthase atoB.

### Enzyme 3: Acetoacetyl-CoA Reductase or Hydroxybutyryl-CoA Dehydrogenase

Recombinant sequences encoding acetoacetyl-CoA reductase or hydroxybutyryl-CoA dehydrogenase may be derived from all prokaryotic organisms, including proteobacterial, archaebacterial, bacteroidal, enterobacterial, spirochetal organisms, and all eukaryotic organisms, including mammalian, insect, fungal and yeast organisms. Preferred examples include, but are not limited to: the R. eutrophus 3-hydroxybutyryl-CoA dehydrogenase phaB (SEQ ID NOs: 17, 18), the C. acetobutylicum acetoacetyl-CoA reductase hbd (SEQ ID NOs: 19, 20).

### Enzyme 4: Crotonase

Recombinant sequences encoding crotonase may be derived from all prokaryotic organisms, including proteobacterial, archaebacterial, bacteroidal, enterobacterial, spirochetal organisms, and eukaryotic organisms, including mammalian, insect, fungal and yeast organisms. Preferred examples include, but are not limited to: the C. acetobutylicum crotonase crt (SEQ ID NOs: 21, 22) or the A. cavaie crotonase phaJ (SEQ ID NOs: 478, 479).

### Enzyme 5: Crotonyl-CoA Reductase or Trans-Enoyl-CoA Reductase

Recombinant sequences encoding crotonyl-CoA reductase or trans-enoyl-CoA reductase may be derived from all prokaryotic organisms, including proteobacterial, archaebacterial, bacteroidal, enterobacterial, spirochetal organisms, and all eukaryotic organisms, including mammalian, insect, fungal and yeast organisms. Preferred examples include, but are not limited to: T. denticola (SEQ ID NOs: 29, 30), E. gracilis (SEQ ID NOs: 31, 32), Burkhoderia mallei, Burkhoderia pseudomallei, Burkhoderia cepacia, Methylobacillus flagellatus, Xylella fastidiosa, Xanthomonas campestris, Xanthomonas cryzae, Pseudomonas putida, Pseudomonas entomophila, Marinomonas sp., Psychromonas ingrahmii, Vibrio alginolyticus, Vibrio parahaemolyticus, Vibrio splendidus, Vibrio sp., Shewanella frigidimarina, Oceanospirillum sp., Aeromonas hydrophila subsp., Serratiae proteamaculans, Saccharophagus degradans, Colwellia psychrerythraea, Reine kea sp., Idiomarina loihiensis, Streptomyces avermitilis, Coxiella burnetii Dugway, Polaribacter irgensii, Flavobacterium johnsoniae, Cytophaga hutchisonii, E. coli, R. eutrophus, A. caviae, or C. acetobutylicum.

The disclosure includes examples for the use of Ters from T. denticola and Euglena gracilis (E. gracilis), the polypeptide sequences of which are 48% homologous.

In a specific embodiment the recombinant sequence encoding the crotonyl-CoA reductase is derived from Streptomyces collinus (S. collinus). In another specific embodiment the recombinant sequence encoding the trans-enoyl-CoA reductase (TER) is derived from T. denticola. In another specific embodiment the crotonyl-CoA reductase is ccr from S. collinus. In another specific embodiment the trans-enoyl-CoA reductase is ter from T. denticola.

### Enzyme 6: Butyraldehyde/Butanol Dehydrogenase

Recombinant sequences encoding the butyraldehyde/butanol dehydrogenase may be derived from all prokaryotic organisms, including proteobacterial, archaebacterial, bacteroidal, enterobacterial, spirochetal organisms, and all eukaryotic organisms, including mammalian, insect, fungal and yeast organisms. Preferred examples include, but are not limited to: the C. acetobutylicum butyraldehyde/butanol dehydrogenases adhE2 (SEQ ID NOs: 33, 34) or aad (SEQ ID NOs: X, Y) and related sequences from Clostridia sp, including but not limited to adhE1, bdhA, bdhB from C. acetobutylicum; and aldH from Clostridium perfringens, Clostridium botulinum A, Clostridium beijerinckii, and Clostridium difficile. In another specific embodiment the butyraldehyde/butanol dehydrogenase is the butyryl-CoA dehydrogenase bcd from C. acetobutylicum.

### Cofactor Specificity

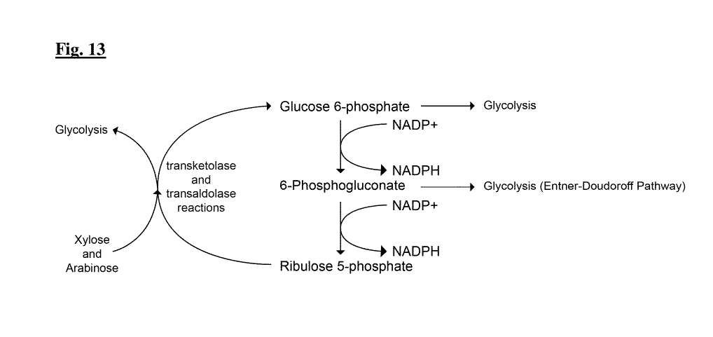

Biomass degradation, and especially the degradation of hemicellulose, yields both C6 sugars such as glucose and C5 sugars such as xylose. Whereas C6 sugars are typically metabolized through the NAMNADH-dependent Embden-Meyerhof-Parnas pathway (the most common glycolytic pathway), C5 sugars are typically metabolized through the Pentose Phosphate Pathway, which is NADP⁺/NADPH-dependent (FIG. 13). NADP⁺/NADPH-dependent enzymes of the Pentose Phosphate Pathway include a glucose dehydrogenase, such as gcd of E. coli, and a 2-keto-D-gluconate reductase, such as tiaE of E. coli. Applicants do not wish to be bound by theory. However, when producing n-butanol from hemicellulose-derived carbon sources it is believed to be beneficial to integrate NADPH-specific enzymes, such as the 3-hydroxybutyryl-CoA dehydrogenase PhaB from R. eutrophus, in the n-butanol synthesis pathway to rebalance the NADP required for continued C5 sugar assimilation.

Because the metabolism of different carbon sources may differently affect cellular NAD⁺/NADH- and NADP⁺/NADPH-redox systems, without wishing to be bound by theory, it is further believed that it is beneficial to tailor recombinant n-butanol synthesis pathways to contain an optimized number of either NAD⁺/NADH-dependent or NADP⁺/NADPH-dependent enzymes. This tailoring allows for an optimal rebalancing of the respective redox systems and ultimately leads to optimized carbon source utilization and n-butanol yields. For example, when metabolizing a hexose-rich carbon source, recombinant cells containing a greater number of NAD⁺/NADH-dependent enzymes are preferred. On the contrary, when metabolizing a pentose-rich carbon source recombinant cells containing a greater number of NADP⁺/NADPH-dependent enzymes are preferred. When metabolizing a carbon source yielding a mix of hexoses and pentoses, such as hemicellulose, recombinant cells containing a mix of NAD⁺/NADH-dependent and NADP⁺/NADPH-dependent enzymes within the recombinant n-butanol pathway are preferred.

In one embodiment of the invention the recombinant n-butanol synthesis pathway uses NADH, but no NADPH. In one specific embodiment, the recombinant n-butanol synthesis pathway (FIG. 1, Enzymes 1-6) uses 4 moles of NADH for the production of one mole of n-butanol. Such a recombinant n-butanol synthesis pathway includes the C. acetobutylicum acetoacetyl-CoA reductase Hbd and the C. acetobutylicum crotonase Crt. In another embodiment of the invention the recombinant n-butanol synthesis pathway uses both NADH and NADPH. In one specific embodiment, the recombinant n-butanol synthesis pathway uses 3 moles of NADH and 1 mole of NADPH for the production of one mole of n-butanol. Such a recombinant n-butanol synthesis pathway includes the R. eutrophus 3-hydroxybutyryl-CoA dehydrogenase PhaB and the A. cavaie crotonase PhaJ. In a preferred embodiment the recombinant n-butanol synthesis pathway using 3 moles of NADH and 1 mole of NADPH includes the acetyl-CoA acetyltransferase PhaA, the R. eutrophus 3-hydroxybutyryl-CoA dehydrogenase PhaB, the A. cavaie crotonase PhaJ and the trans-enoyl-coA reductase Ter from T. denticola.

### Coenzyme A Synthesis

In one embodiment the recombinant cell further contains recombinant sequences encoding one or more enzymes of the coenzyme A biosynthesis pathway.

In one embodiment the recombinant cell further contains a recombinant sequence encoding a pantothenate kinase catalyzing the conversion of pantothenate to 4′-phosphopantothenate. In one specific embodiment the pantothenate kinase is derived from E. coli. In another specific embodiment the pantothenate kinase is PanK/CoaA (SEQ ID NOs: 455, 456), or CoaX SEQ ID NOs: 457, 458).

In another embodiment the recombinant cell further contains a recombinant sequence encoding a phosphopantothenoylcysteine synthetase catalyzing the conversion of 4′-phosphopantothenate to 4′-phosphopantothenoylcysteine. In a specific embodiment the phosphopantothenoylcysteine synthetase is derived from E. coli. In another specific embodiment the phosphopantothenoylcysteine synthetase is Ppcs or CoaB (SEQ ID NOs: 459, 460).

In another embodiment the recombinant cell further contains a recombinant sequence encoding phosphopantothenonylcysteine decarboxylase catalyzing the conversion of 4′-phosphopantothenoylcysteine to 4′-phosphopantetheine. In a specific embodiment the phosphopantothenonylcysteine decarboxylase is derived from E. coli. In another specific embodiment the phosphopantothenonylcysteine decarboxylase is Ppcdc or CoaC (SEQ ID NOs: 459, 460).

In another embodiment the recombinant cell further contains a recombinant sequence encoding phosphopantetheine adenylyl transferase catalyzing the transfer of an adenylyl group from ATP to 4′-phosphopantetheine. In a specific embodiment the phosphopantetheine adenylyl transferase is derived from E. coli. In another specific embodiment the phosphopantetheine adenylyl transferase is Ppat or CoaD (SEQ ID NOs: 461, 462).

In another embodiment the recombinant cell further contains a recombinant sequence encoding dephosphocoenzyme A kinase catalyzing the phosphorylation of dephospho-CoA. In a specific embodiment the dephosphocoenzyme A kinase is derived from E. coli. In another specific embodiment the dephosphocoenzyme A kinase is CoaE (SEQ ID NOs: 463, 464).

Recombinant sequences encoding pantothenate kinase, phosphopantothenoylcysteine synthetase, phosphopantothenonylcysteine decarboxylase, phosphopantetheine adenylyl transferase, or dephosphocoenzyme A kinase may be derived from all prokaryotic organisms, including proteobacterial, archaebacterial, bacteroidal, enterobacterial, spirochetal organisms, and all eukaryotic organisms, including mammalian, insect, fungal and yeast organisms.

### Competing Pathways

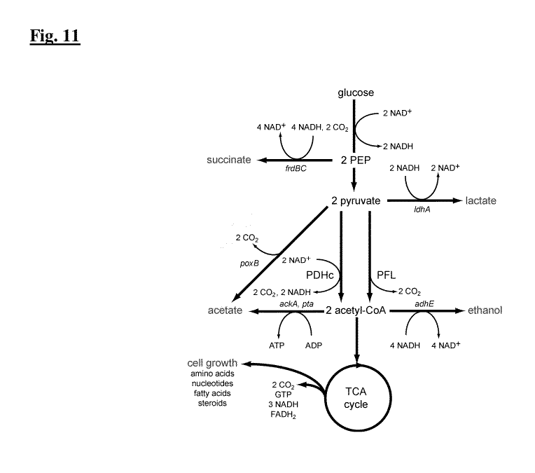

In one embodiment of the invention the recombinant cell further contains mutations reducing or eliminating the activity of enzymes in pathways that utilize pyruvate or acetyl-CoA to synthesize products other than n-butanol (FIG. 11). In one specific embodiment enzyme activities are reduced or eliminated in a pathway synthesizing lactate from pyruvate. In another specific embodiment enzyme activities are reduced or elimimanted in a pathway synthesizing acetate from pyruvate. In another specific embodiment enzyme activities are reduced or eliminated in a pathway synthesizing acetate from acetyl-CoA. In another specific embodiment enzyme activities are reduced or eliminated in a pathway synthesizing ethanol from acetyl-CoA.

In one embodiment the recombinant cell contains a lactate dehydrogenase that catalyzes the conversion of pyruvate to lactate with reduced or eliminated activity. In a specific embodiment the lactate dehydrogenase is ldhA from E. coli. In another embodiment the recombinant cell contains a pyruvate oxidase that catalyzes the conversion of pyruvate to acetate with reduced or eliminated activity. In a specific embodiment the pyruvate oxidase is poxB from E. coli. In another embodiment the recombinant cell contains an alcohol dehydrogenase that catalyzes the conversion of acetyl-CoA to ethanol with reduced or eliminated activity. In a specific embodiment the alcohol dehydrogenase is adhE from E. coli. In another embodiment the recombinant cell contains an acetate kinase that catalyzes the conversion of acetyl-CoA to acetate with reduced or eliminated activity. In a specific embodiment the acetate kinase is ackA. In another embodiment the recombinant cell contains a phosphotransacetylase that catalyzes the conversion of acetyl-CoA to acetate with reduced or eliminated activity. In a specific embodiment the phosphotransacetylase is pta. In another embodiment the recombinant cell contains a fumarate dehydrogenase that catalyzes the conversion of succinate to fumarate with reduced or eliminated activity. In a specific embodiment the phosphotransacetylase is frd from E. coli.

The activity of an enzyme having reduced or eliminated activity may be reduced by 10%, 20%, 30%, 40%, 50%, 60%, 70%, 80%, 90%, or 100% relative to a wild type enzyme, the activity of which is not reduced. Mutatations reducing or eliminating the activity of enzymes may include point mutations that cause amino acid changes in the enzymes, deletion mutations, nonsense mutations, frameshift mutations, sequence duplications or inversions and insertions. Mutations may be introduced in a targeted or non-targeted manner. Mutations may be introduced by molecular biology means, such as homologous recombinations, antisense technologies or RNA interference, or by chemical means, such as treatments with DNA intercalators or DNA methylating agents.

In one embodiment the recombinant cell is a yeast cell. In a specific embodiment the yeast cell further contains mutations reducing or eliminating the activity of enzymes in pathways that utilize pyruvate or acetyl-CoA to synthesize products other than n-butanol. In another specific embodiment the enzymes may include the alcohol dehydrogenase adh1, the NAD-dependent glycerol-3-phosphate dehydrogenases gpd1 or gpd2, the NADP-dependent glutamate dehydrogenase gdh1, the aquaglyceroporin fps1, the pyruvate decarboxylases pdc1, pdc2, pdc3, pdc4, and pdc5, the acetyl-CoA synthetases acs1 and acs2, and the acetaldehyde dehydrogenases ALDH1, ADLH2, ALDH3, ALDH4, ALDH5, ALDH6.

In another specific embodiment the recombinant cell further contains recombinant sequences encoding the glutamate synthase glt1 or the glutamine synthetase gln1.

### Cells

Recombinant cells of the invention may include all prokaryotic-including proteobacterial, archaebacterial, bacteroidal, enterobacterial, spirochetal- and eukaryotic-including mammalian, insect, fungal and yeast-cell types. Preferred embodiments of the invention include, but are not limited to E. coli cells, Zymomonas mobilis (Z. mobilis) cells, Bacillus subtilis (B. subtilis) cells, yeast cells including S. cerevisiae cells and S. pombe cells, cyanobacterial cells such as Synechocystis sp. and Synechococcus sp., photosynthetic cells such as Rhodospirillum sp., solvent producing cells such as Clostridium sp. (including but not limited to Clostridium acetobutylicum and Clostridium beijerinckii), chemoautotrophic cells such as Ralstonia sp., in general and Ralstonia eutrophus in particular, aromatic-degrading cells such as Pseudomonas sp. and Rhodococcus sp., thermophilic cells such as Thermoanaerobacterium saccharolyticum (T. saccharolyticum) and Thermotoga sp., cellulytic cells such as Trichoderma reesei (T. reesei) cells, and Aspergillus niger (A. niger) cells, and lignocellulytic cells such as Phanerochaete chrysosporium (P. chrysosporium), CHO cells, SF9 cells.

### General Methods

Metabolites and products formed as part of the recombinant biofuel pathway can be identified and quantified using standard HPLC chromatography and mass spectrometry techniques. Enzymatic activities can be determined using traditional spectrophotometric activity assays relying on the detection of NAD(P)H cofactor consumption.

The nucleic acids may be synthesized, isolated, or manipulated using standard molecular biology techniques such as those described in Sambrook, J. et al. 2000. Molecular Cloning: A Laboratory Manual (Third Edition). Techniques may include cloning, expression of cDNA libraries, and amplification of mRNA or genomic DNA.

The nucleic acids of the present disclosure, or subsequences thereof, may be incorporated into a cloning vehicle comprising an expression cassette or vector. The cloning vehicle can be a viral vector, a plasmid, a phage, a phagemid, a cosmid, a fosmid, a bacteriophage, or an artificial chromosome. The viral vector can comprise an adenovirus vector, a retroviral vector, or an adeno-associated viral vector. The cloning vehicle can comprise a bacterial artificial chromosome (BAC), a plasmid, a bacteriophage P1-derived vector (PAC), a yeast artificial chromosome (YAC), or a mammalian artificial chromosome (MAC).

The nucleic acids may be operably linked to a promoter. The promoter can be a viral, prokaryotic, or eukaryotic promoter. The promoter can be a constitutive promoter, an inducible promoter, a tissue-specific promoter, or an environmentally regulated or a developmentally regulated promoter.

Methods for Producing n-Butanol

In one embodiment of the invention the method for the production of n-butanol includes the step of growing a recombinant cell of the invention in the presence of a suitable carbon source.

Suitable carbon sources may include, but are not limited to glucose, glycerol, sugars, starches, and lignocellulosics, including but not limited to glucose derived from cellulose and C₅ sugars derived from hemicellulose, such as xylose.

In one specific embodiment the recombinant cell of the invention is grown under aerobic conditions. In another specific embodiment the recombinant cell of the invention is grown under microaerobic conditions. In another specific embodiment the recombinant cell of the invention is grown under anaerobic conditions. In another specific embodiment the recombinant cell of the invention is grown under conditions wherein it produces more n-butanol under anaerobic conditions than under aerobic or microaerobic conditions. In another specific embodiment the recombinant cell of the invention is grown under conditions wherein it produces more total levels of n-butanol and ethanol under anaerobic conditions than under aerobic or microaerobic conditions. In another specific embodiment the recombinant cell of the invention is grown under anaerobic conditions wherein it produces near quantitative yields of n-butanol. In another specific embodiment the recombinant cell of the invention is grown under anaerobic conditions wherein it produces near quantitative yields of n-butanol and ethanol.

In one specific embodiment the recombinant cell of the invention is grown under aerobic conditions wherein it produces elevated levels of n-butanol compared to a wild-type cell grown under aerobic conditions. Total levels of n-butanol produced by the recombinant cell of the invention under aerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 100,000-fold compared to the n-butanol levels produced by a wild-type cell under aerobic conditions. In specific embodiments the recombinant cell of the invention is grown under aerobic conditions wherein it produces at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L n-butanol.

In one specific embodiment the recombinant cell of the invention is grown under aerobic conditions wherein it produces elevated total levels of n-butanol and ethanol compared to a wild-type cell grown under aerobic conditions. Total levels of n-butanol and ethanol produced by the recombinant cell of the invention under aerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 100,000-fold compared to the total levels of n-butanol and ethanol produced by a wild-type cell under aerobic conditions. In specific embodiments the recombinant cell of the invention is grown under aerobic conditions wherein it produces total levels of n-butanol and ethanol of at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L.

In one specific embodiment the recombinant cell of the invention is grown under anaerobic conditions wherein it produces elevated levels of n-butanol compared to a wild-type cell grown under anaerobic conditions. Total levels of n-butanol produced by the recombinant cell of the invention under anaerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 100,000-fold compared to the n-butanol levels produced by a wild-type cell under anaerobic conditions. In specific embodiments the recombinant cell of the invention is grown under anaerobic conditions wherein it produces at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L n-butanol.

In one specific embodiment the recombinant cell of the invention is grown under anaerobic conditions wherein it produces elevated total levels of n-butanol and ethanol compared to a wild-type cell grown under anaerobic conditions. Total levels of n-butanol and ethanol produced by the recombinant cell of the invention under anaerobic conditions may be elevated by 5%, 10%, 20%, 30%, 40%, 50%, 60%, 70%, 80%, 100%, 3-fold, 10-fold, 30-fold, 100-fold, 300-fold, 1,000-fold, 3,000-fold, 10,000-fold, 30,000-fold, 100,000-fold, 300,000-fold or 100,000-fold compared to the total levels of n-butanol and ethanol produced by a wild-type cell under anaerobic conditions. In specific embodiments the recombinant cell of the invention is grown under anaerobic conditions wherein it produces total levels of n-butanol and ethanol of at least 0.01 g/L, at least 0.03 g/L, at least 0.1 g/L, at least 0.3 g/L, at least 1.0 g/L, at least 1.5 g/L, at least 2.0 g/L, at least 2.5 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 15.0 g/L, at least 20.0 g/L, at least 30.0 g/L, at least 50.0 g/L, or at least 75.0 g/L.

The methods described herein can be practiced in combination with other methods useful for the production of n-butanol, such as methods for the conversion of lignocellulosic materials into biofuels.

For example, plant material may be subjected to pretreatment including ammonia fiber expansion (AFEX), steam explosion, treatment with alkaline aqueous solutions, acidic solutions, organic solvents, ionic liquids (IL), electrolyzed water, phosphoric acid, and combinations thereof. Pretreatments that remove lignin from the plant material may increase the overall amount of sugar released from the hemicellulose.

Because hemicellulose degradation yields both C6 sugars (e.g., glucose) and C5 sugars (e.g., xylose) a combination of recombinant n-butanol biosynthesis pathways with optimized recombinant glycolysis pathways (for C6 sugar assimilation) or optimized recombinant pentose phosphate pathways (for C5 sugar assimilation) may be useful for the achievement of optimal biomass utilization and n-butanol yields.

### Preferred Embodiments

In one preferred embodiment of the invention the recombinant cell contains recombinant sequences encoding the pyruvate decarboxylase Pdc from Z. mobilis, the acylating aldehyde dehydrogenase EutE from E. coli, the keto-thiolase PhaA from R. eutrophus, the hydroxybutyryl-CoA dehydrogenase Hbd from C. acetobutylicum, the crotonase Crt from C. acetobutylicum, the crotonyl-CoA reductase Ter from T. denticola, and the alcohol dehydrogenase AdhE2 from C. acetobutylicum. In another preferred embodiment the recombinant cell contains recombinant sequences encoding the pyruvate:flavodoxin/ferredoxin-oxidoreductase YdbK from E. coli, the keto-thiolase PhaA from R. eutrophus, the hydroxybutyryl-CoA dehydrogenase Hbd from C. acetobutylicum, the crotonase Crt from C. acetobutylicum, the crotonyl-CoA reductase Ter from T. denticola, and the alcohol dehydrogenase AdhE2 from C. acetobutylicum. In another preferred embodiment the recombinant cell is a S. cerevisiae cell, an E. coli cell, a C. acetobutylicum cell, or a C. beijerinckii cell.

In another preferred embodiment the recombinant cell further contains a recombinant sequence encoding a component of an acetyl-CoA synthesis pathway, including pantothenate kinase (PanK, CoaA, CoaX), phosphopantothenoylcysteine synthetase (Ppcs, CoaB), phosphopantothenonylcysteine decarboxylase (Ppcdc, CoaC), and phosphopantetheine adenylyl transferase (Ppat, CoaD), and dephosphocoenzyme A kinase (CoaE).

In another preferred embodiment the recombinant cell further contains reduced or eliminated activities of at least one enzyme of a biosynthesis pathways utilizing pyruvate or acetyl-CoA for other purposes than n-butanol biosynthesis, such as lactate dehydrogenase, pyruvate oxidase, alcohol dehydrogenase, acetate kinase, or phosphotransacetylase.

In another preferred embodiment a preferred recombinant cell of the invention is grown in the presence of a suitable carbon source. In another preferred embodiment the preferred cell of the invention is grown under anaerobic conditions. In another preferred embodiment the preferred cell of the invention is grown under conditions wherein the cell produces total levels of n-butanol and ethanol of at least 5.0 g/L.

### EXAMPLES

The following Examples are merely illustrative and are not meant to limit any aspects of the present disclosure in any way.

### Summary of Examples

Example 1:Production of n-butanol in recombinant E. coli

Example 2: Identification of bottleneck in recombinant n-butanol synthesis pathway

Example 3: Ter increases n-butanol production in recombinant cells

Example 4: Elevation of PDH and PFOR activities further increase n-butanol yields

Example 5: Efficient production of n-butanol in a recombinant cell

Example 6: Construction of a recombinant S. cerevisiae cell for n-butanol production

### Materials and Methods.

Terrific Broth (TB), LB Broth Miller (LB), LB Agar Miller, sulfuric acid and glycerol were purchased from EMD Biosciences (Darmstadt, Germany). Isopropyl β-D-1-thiogalactopyranoside (IPTG) D-glucose, Dithiothreitol (DTT), Tris-HCl, phenylmethanesulfonyl fluoride (PMSF), carbenicillin (Cb), ammonium acetate, streptomycin sulfate and HPLC-grade acetonitrile were purchased from Fisher Scientific (Pittsburgh, Pa.). L-arabinose, chloramphenicol (Cm), kanamycin (Km), coenzyme A (CoASH), acetyl-CoA, acetoacetyl-CoA, crotonyl-CoA, butyryl-CoA, butyraldehyde, N,N,N′,N′-Tetramethylethylenediamine (TEMED), NADH, NADPH, and NAD were purchased from Sigma-Aldrich (St. Louis, Mo.). Polyacrylamide, Protein Assay reagent, electrophoresis grade sodium dodecyl sulfate (SDS), and ammonium persulfate were purchased from Bio-Rad Laborabories (Hercules, Calif.). All PCR amplifications were carried out with Phusion polymerase (New England BioLabs; Ipswich, Mass.), unless otherwise noted. Deoxynucleotides (dNTPs) and Platinum Taq High-Fidelity polymerase (Pt Taq HF) were purchased from Invitrogen (Carlsbad, Calif.). All restriction enzymes, antarctic phosphatase, polynucleotide kinase, T4 Polymerase and T4 DNA ligase were purchased from New England Biolabs (Ipswich, Mass.). DNA was isolated using the QIAprep Spin Miniprep Kit, QIAquick PCR Purification Kit, and QIAquick Gel Extraction Kit (QIAGEN; Valencia, Calif.) as appropriate. Oligonucleotides were purchased from Integrated DNA Technologies (Coralville, Iowa) and resuspended at a stock concentration of 100 μM in 10 mM Tris-HCl, pH 8.5. Codon optimization and back-translation were carried out using Gene Designer 2.0 (DNA 2.0; Menlo Park, Calif.). All synthetic genes and inserts were sequenced using the sequencing primers for the appropriate gene(s) following plasmid construction by the UC Berkeley Sequencing Facility, Sequetech (Mountain View, Calif.), or Quintara Biosciences (Berkeley, Calif.). All absorbance readings were taken on a DU-800 spectrometer (Beckman-Coulter; Fullerton, Calif.) or a SpectraMax M2 plate reader (Molecular Devices; Toronto, Canada).

### Bacterial Strains.

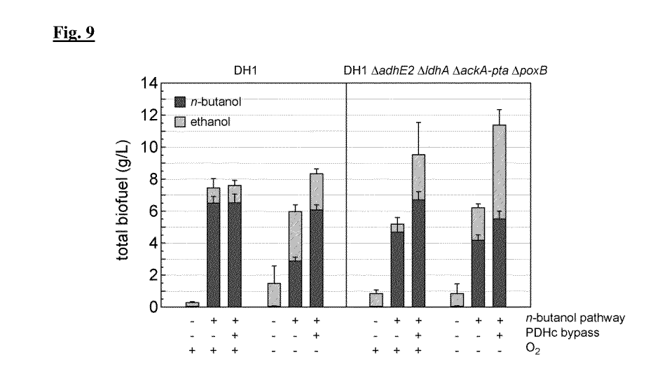

E. coli DH10B-T1R, DH10B-T1R(de3), DH1, DH1(de3), and BL21(de3), and were used for protein and n-butanol production studies. DH10B-T1R and DH1 were lysogenized using λDE3 Lysogenization Kit from Novagen (San Diego, Calif.). Additional strain optimization in E. coli DH1 was achieved by knocking out metabolic genes to divert carbon flux from organic acid metabolites to the synthetic butanol pathway (Table 1, FIG. 9).

### Cell Culture.

E. coli strains were transformed by electroporation using the appropriate plasmids. A single colony from a fresh transformation was then used to seed an overnight culture grown in Terrific Broth (TB) supplemented with 0.5% glucose and appropriate antibiotics at 37° C. in a rotary shaker (200 rpm). Antibiotics were used at a concentration of 50 μg/mL for strains with a single resistance marker. For strains with multiple resistance markers, kanamycin (Km) and chloramphenicol (Cm) were used at 25 μg/mL and carbenicillin (Cb) was used at 50 μg/mL.

### Example 1

### Production of n-Butanol in E. coli

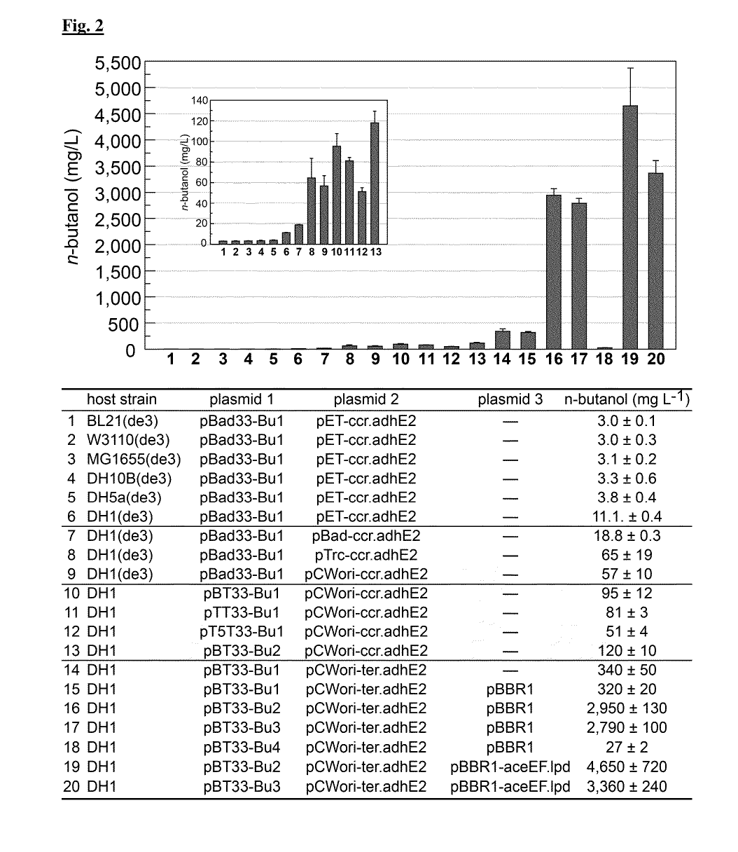

A recombinant pathway for n-butanol synthesis in E. coli was constructed in the form of a two plasmid system in E. coli BL21(de3) cells comprising the R. eutrophus genes phaA and phaB, the C. acetobutylicum genes crt and adh2 and the S. cinnamonensis gene ccr (FIG. 2). Although n-butanol formation could be observed by gas chromatography-mass spectometry, the titer achieved in E. coli BL21(de3) cells was low (˜2 mg/L).

### Gene Synthesis

Synthetic genes encoding PhaA (SEQ ID NO 15), PhaB (SEQ ID NO 16), Crt (SEQ ID NO 21), Ccr (SEQ ID NO 23), and AdhE2 (SEQ ID NO 33) were optimized for E. coli class II codon usage and obtained from Epoch Biosciences (Sugar Land, Tex.). Gene2Oligo (http://berry.engin.umich.edu/gene2oligo) was used to convert the gene sequence into primer sets using default optimization settings (Gene Construction Primers: Ter (E. gracilis)—SEQ ID NOs 45-112; Ter (T. denticola)—SEQ ID NOs 113-184; Ccr (S. cinnamonensis)—SEQ ID NOs 185-260; Hbd (C. acetobutylicum)—SEQ ID NOs 261-314). To assemble the synthetic gene, each primer was added at a final concentration of 1 μM to the first PCR reaction (50 μL) containing 1×Pl Taq HF buffer (20 mM Tris-HCl, 50 mM KCl, pH 8.4), MgSO₄ (1.5 mM), dNTPs (250 μM each), and Pt Taq HF (5 U). The following thermocycler program was used for the first assembly reaction: 95° C. for 5 min; 95° C. for 30 s; 55° C. for 2 min; 72° C. for 10 s; 40 cycles of 95° C. for 15 s, 55° C. for 30 s, 72° C. for 20 s plus 3 s/cycle; these cycles were followed by a final incubation at 72° C. for 5 min. The second assembly reaction (50 μL) contained 16 μL of the unpurified first PCR reaction with standard reagents for Pt Taq HF. The thermocycler program for the second PCR was: 95° C. for 30 s; 55° C. for 2 min; 72° C. for 10 s; 40 cycles of 95° C. for 15 s, 55° C. for 30 s, 72° C. for 80 s; these cycles were followed by a final incubation at 72° C. for 5 min. The second PCR reaction (16 μL) was transferred again into fresh reagents and run using the same program. Following gene construction, the DNA smear at the appropriate size was gel purified and used as a template for the rescue PCR (50 μL) with Pt Taq HF and rescue primers (TdTer F1 and R1) under standard conditions. The resulting rescue product was either inserted directly in the appropriate vector or first cloned into pCR2.1-TOPO using a TOPO TA Cloning Kit from Invitrogen.

### Construction of Plasmids

Standard molecular biology techniques were used to carry out plasmid construction using E. coli DH10B-T1R as the cloning host. Primers are listed in SEQ ID NOs 315-334. Annealed inserts were generated by phosphorylating each primer (1.5 pmol) individually with polynucleotide kinase in T4 DNA ligase buffer followed by incubation at 37° C. for 30 min and heat inactivation at 65° C. for 20 min. The phosphorylated primers were then mixed in 1× annealing buffer (100 mM NaCl, 50 mM HEPES, pH 7.4) and annealed using the following program and used immediately once the reaction reached 25° C.: 90° C. for 4 min, 70° C. for 10 min, ramped to 37° C. at 0.5° C./s, 37° C. for 15 min, ramped to 25° C. at 0.5° C./s.

pBT33-phaAB-crt. The phaAB operon was amplified from pCR2.1-phaA2.phaB using the phaA2 F2 and phaB R2 primers and inserted into the SacI-XbaI restriction sites of pBAD33 to generate pBAD33-phaAB. The pTrc99a-crt cloning intermediate was made by inserting the synthetic crt gene into the NcoI-XmaI restriction sites of pTrc99a using the crt F2 and crt R2 primers to amplify the insert. The resulting PTrc.crt.rrnB cassette was amplified from pTrc99a-crt using the pTrc99a F4 and pTrc99a R4 primers and inserted non-directionally into the BglI site of pBAD33-phaAB to produce pBT33-phaABcrt. Sequencing showed the coding strand of the phaAB operon was on the same strand as the crt gene. pBT33-phaB-hbd. The pCR2.1-phaA.hbd cloning intermediate was constructed by amplification of the synthetic hbd gene from pCR2.1-hbd with the hbd F1 and hbd R1 primers and insertion into the EcoRIHindIII restriction sites of pCR2.1-phaA2.phaB. The phaAB operon of pBT33-phaAB-crt was then replaced with a new multiple cloning site by digestion with NdeI and XhoI and insertion of a linker using sequence and ligation independent cloning (SLIC) (Li and Elledge, 2007, Nature Methods. 4, 251-56). The insert was made by amplifying the rrnB terminator from pBAD33 using primers rrnB SLIC F1 and rrnB SLIC R1. The amplified fragment and digested vector were independently treated with 0.5 U T4 polymerase for 30 min and the reaction was quenched with the addition of dATP. The insert and vector were incubated in 1× ligation buffer for 30 min at 37° C. and transformed immediately.

pCWOri-ccr.adhE2. pCWOri-ccr.adhE2 was made by inserting the ccr-adhE2 operon from pET29accr. adhE2 into the NdeI-HindIII sites of pCWOri. The primers used to amplify the operon were ccr F1 and adhE2 R1.

In Vivo Production of n-Butanol

For production of n-butanol production in baffled flasks, the overnight cultures were grown for 12-16 h and used to inoculate TB (50 mL) with either 2% glucose or 2% glycerol replacing the standard glycerol supplement and appropriate antibiotics in a 250 mL-baffled flask to a starting OD600=0.05. The cultures were grown at 37° C. in a rotary shaker (200 rpm) and induced with IPTG (1.0 mM) and L-arabinose (0.2%) when appropriate at OD600=0.35-0.45. At this time the growth temperature was reduced to 30° C. Upon induction and following all daily samplings, flasks were sealed with Parafilm M (Pechiney Plastic Packaging, Chicago, Ill.). For production of n-butanol production in culture tubes, the overnight cultures were grown for 22-26 h and used to inoculate (1%, 50 μL) precultures in TB with 0.5% glucose (5 mL). After incubation at 37° C. in rotary shaker (250 rpm) for 16 h, precultures were back-diluted 8 to OD600=0.4 in TB with 2.5% glucose replacing the standard glycerol supplement (5 mL) in anaerobic tubes (20 mm; Bellco Glass; Vineland, N.J.) and induced with IPTG (1.0 mM) and L-arabinose (0.2%). The growth temperature was then reduced to 30° C. and the culture tubes sealed with aluminum seals using butyl rubber septa (Bellco Glass) unless otherwise noted. For anaerobic growth, the headspace of the cultures was deoxygenated with Ar gas after backdilution and induction. Semi-anaerobic growth was performed with cultures in sealed tubes without degassing with Ar and aerobic growth was performed in unsealed tubes. Extraction and quantification of n-butanol. Samples (2 mL) were removed from cell culture and cleared of biomass by centrifugation at 20817×g for 2 min using an Eppendorf 5417R centrifuge (Hamburg, Germany). The supernatant or cleared media sample was then mixed 1:1 with an aqueous solution containing the isobutanol internal standard (1000 mg/L). These samples were then analyzed on a Trace GC Ultra (Thermo Scientific; Waltham, Mass.) using an HP-5MS column (0.25 mm×30 m, 0.25 μM film thickness, J & W Scientific). The oven program was as follows: 75° C. for 3 min, ramp to 300° C. at 45° C./min, 300° C. for 1 min. n-Butanol was quantified using by flame ionization detection (FID) (using flow of 350 ml/min air, 35 ml/min H₂, and 30 ml/min He). Samples containing n-butanol levels below 500 mg/L were then re-quantified with a DSQII single-quadrupole mass spectrometer (Thermo Scientific; Waltham, Mass.) using single ion monitoring (m/z 41 and 56) concurrent with full scan mode (m/z 35-80) for samples with n-butanol levels lower than 500 mg/L. Samples were quantified relative to a standard curve of 2, 5, 10, 25, 50, and 100 mg/L n-butanol for MS detection or 62.5, 125, 250, 500, 1000, 2000, 4000 mg/L n-butanol for FID detection. Standard curves were prepared freshly during each run and normalized for injection volume using the internal isobutanol standard

### Example 2

### Identification of Bottleneck in Recombinant n-Butanol Synthesis Pathway

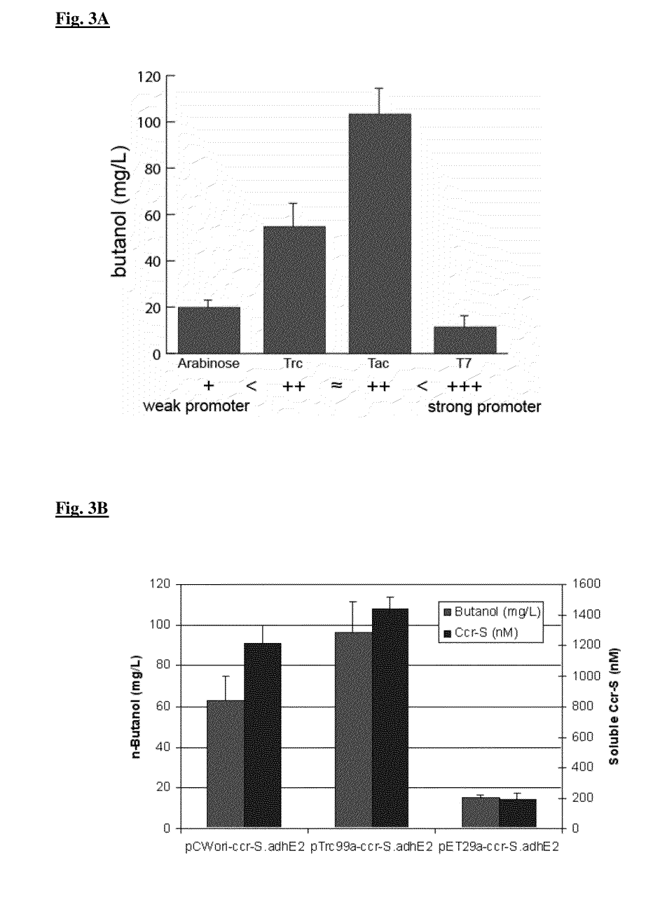

The initial n-butanol yields obtained with the recombinant cellular system of Example 1 were subsequently improved ˜60-fold by promoter and host cell optimization (FIGS. 2 and 3A).

A correlation was observed between n-butanol yields and solubility of the Ccr protein, which pointed to a bottleneck in the n-butanol biosynthesis pathway at the conversion step of crotonyl-CoA to butyryl-CoA (FIG. 3B).

### Construction of Plasmids

pBAD33-ccr.adhE2. The ccr-adhE2 operon was amplified from pET29a-ccr.adhE2 using the ccr F1 and adhE2 R17 primers and inserted into the NdeI-SalI sites of pBAD33-phaAB, the insert was digested using NdeI and XhoI.

pTrc99a-ccr.adhE2. pTrc99a-ccr.adhE2 was made by inserting the ccr-adhE2 operon from pET29accr.adhe2 into the NcoI-SacI sites. The primers used to amplify the operon were ccr F15 and adhE2 R2.

pCWOri-ter.adhE2. The ter gene was amplified from pET16b-His-ter with TdTer F1 and TdTer R102 and inserted directly into the NdeI-EcoRI restriction sites of pCWOri-ccr. adhE2.

pET29a-ccr.adhE2. The ccr gene was amplified using the ccr F1 and ccr R2 primers and inserted into the NdeI-EcoRI sites of pET29a. pET29-ccr.adhE2 was constructed by insertion of the adhE2 gene into the EcoRI-SacI restriction sites of pET29a-ccr after amplification using the adhE2 μl and adhE2 R2 primers.

### Example 3

### Ter Increases n-Butanol Production in Recombinant Cells

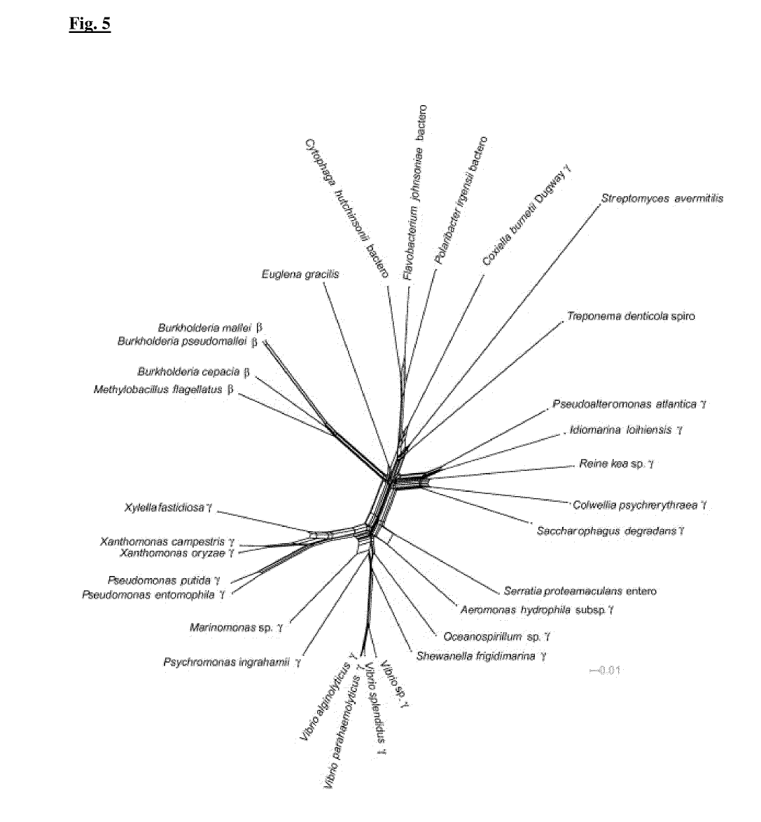

In an experiment similar to Example 1, the replacement of the S. cinnamonensis gene ccr for ter genes from E. gracilis and T. denticola resulted in significantly increased n-butanol yields, where the recombinant biosynthesis pathway further comprised the R. eutrophus gene phaA, and the C. acetobutylicum genes hbd, crt and adh2 (FIG. 5). This experiment thus demonstrates that the incorporation of Ter enzymes into the recombinant biosynthesis pathway for n-butanol relieves a bottleneck at the stage of crotonyl-CoA to butyryl-CoA conversion.

### Example 4

### Elevation of PDH and PDHc Bypass Activities Further Increase n-Butanol Yields

Acetyl-CoA is the building block for the production of advanced fuels ranging from short-, medium-, and long-chain length fatty alcohols, fatty acids, fatty acid esters, and alkanes. A major challenge in the production of these molecules is the bottleneck from the endpoint of glycolysis, the conversion of pyruvate to acetyl-CoA. Four classes of enzymes were identified that can relieve this bottleneck: pyruvate dehydrogenase PDH, PDHc bypass comprised of two enzymes (pdc and eutE), E. coli pyruvate formate oxido-reductace (PFOR), and E. coli pyruvate formate lyase with C. boidinii formate dehydrogenase (pfl and fdh).

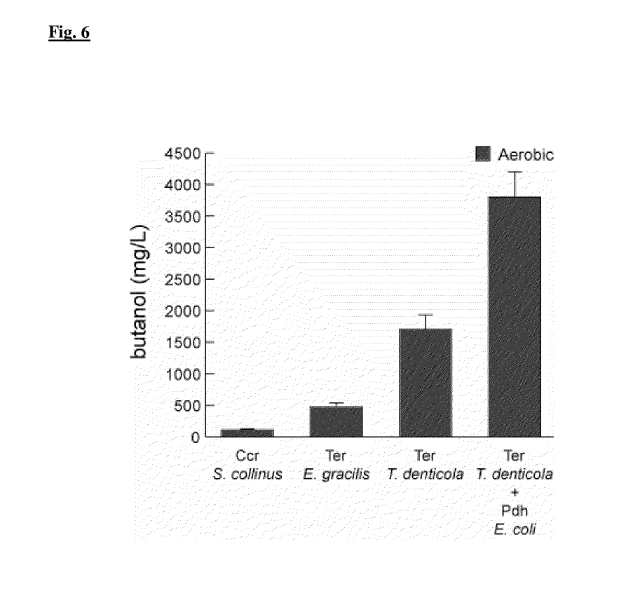

In an experiment similar to Example 4, the elevation of PDH activity further increased n-butanol yields beyond the yields observed in the presence of Ter alone (FIGS. 5 and 6). This finding demonstrates that a second bottleneck existed in the n-butanol biosynthesis pathway at the initial conversion of pyruvate to acetyl-CoA. Increasing the concentration of acetyl-CoA by increasing the turnover of pyruvate relieved this second bottleneck and resulted in higher n-butanol yields.

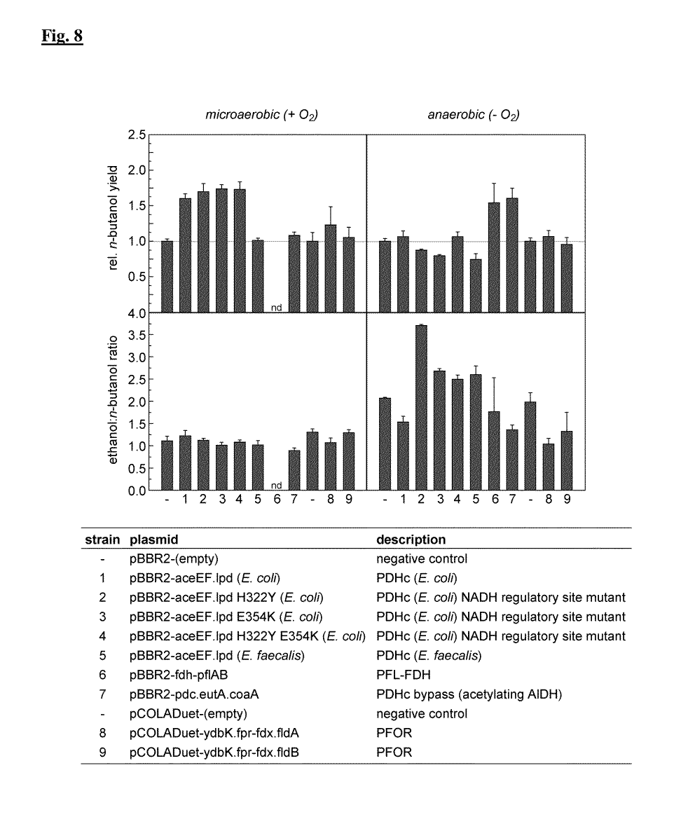

The third route to generate acetyl-CoA from pyruate is catalyzed by PDHc bypass that is composed of two enzymes, pyruvate decaroboxylase and acetylating aldehyde dehydrogenase. Acetaldehyde is generated by pyruvate decarboxylase from pyruvate and then oxidized to acetyl-CoA, coupled with the reduction of NAD+ to balance the reducing equivalent required for butanol synthesis. In the presence of these enzymes, and under anaerobic conditions, n-butanol yield can increase by 50% (FIG. 8).

### Example 5

### Efficient Production of n-Butanol in a Recombinant Cell

Through the use of Ter from T. denticola and overexpression of the E. coli pyruvate dehydrogenase complex or the pyruvate decarboxylase of Z. mobilis and the acetylating aldehyde dehydrogenase of E. coli in a pathway otherwise comprising the R. eutrophus gene phaA, and the C. acetobutylicum genes hbd, crt and adh2 it was possible to engineer a highly efficient recombinant cell for the production of n-butanol.

TABLE 1
Knockout E. coli DH1 host strains for the
production of n-butanol.

| Strain | Genotype |
|---|---|
| E. coli | endA1 recA1 gyrA96 thi-1 glnV44 relA1 hsdR17(rK− mK+) λ− |
| DH1 | |
| MC001 | E. coli DH1 ΔadhE |
| MC002 | E. coli DH1 ΔadhE, ΔldhA |
| MC003 | E. coli DH1 ΔadhE, ΔldhA, ΔackA-pta |
| MC004 | E. coli DH1 ΔadhE, ΔldhA, ΔpoxB |
| MC005 | E. coli DH1 ΔadhE, ΔldhA, ΔackA-pta, ΔpoxB |
| MC006 | E. coli DH1 ΔadhE, ΔldhA, ΔackA-pta, ΔpoxB, ΔfrdBC |

### Example 6

### n-Butanol Production in a Recombinant S. Cerevisiae Cell

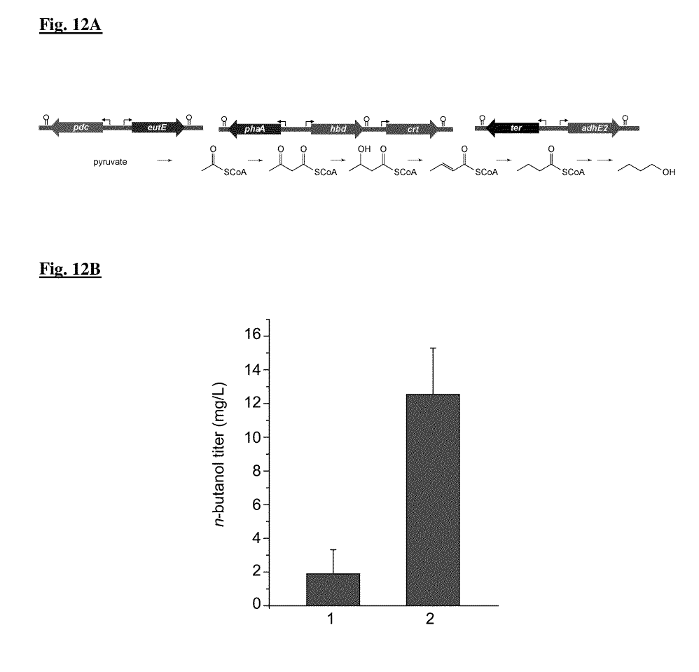

S. cerevisiae is another preferred host for a recombinant n-butanol production pathway and well suited to support industrial fuel production. The preferred recombinant n-butanol synthesis pathway was inserted into S. cerevisiae (FIG. 12A). The recombinant pathway includes the pyruvate decarboxylase Pdc from Z. mobilis, the acylating aldehyde dehydrogenase EutE from E. coli, the keto-thiolase PhaA from R. eutrophus, the hydroxybutyryl-CoA dehydrogenase Hbd from C. acetobutylicum, the crotonase Crt from C. acetobutylicum, the crotonyl-CoA reductase Ter from T. denticola, and the alcohol dehydrogenase AdhE2 from C. acetobutylicum (FIG. 12A). The DNA constructs shown in FIG. 12A for both plasmid-based and chromosomal gene expression were made using standard methods described above and one-step isothermal DNA assembly as described by Gibson, et al., Nat. Methods. (2009) 6, p. 343.

To optimize production of n-butanol, pyruvate decarboxylase pdc (mutant cell: Δpdc) and the alcohol dehydrogenase adh1 (mutant cell: Δadh1) were targeted for deletion in S. cerevisiae because these enzymes are involved in competing, acetyl-CoA consuming pathways other than n-butanol production. (See also FIG. 11 for analogous E. coli pathways). Wild-type S. cerevisiae as well as Δpdc and Δadh1 strains bearing a plasmid-based n-butanol genetic system were prepared using standard molecular biology techniques. Recombinant S. cerevisiae cells with the preferred n-butanol pathway were shown to produce at least 10 mg/L n-butanol. For example, a Δadh1 mutant cell, S. cerevisiae BY4741Δadh, containing the n-butanol production pathway (FIG. 12A) was shown to produce greater than 12 mg/L n-butanol (FIG. 12B, column 2), whereas the background level of n-butanol production of S. cerevisiae BY4741Δadh was only about 2 mg/L (FIG. 12B, column 1).

What is claimed:

1. A recombinant cell for the synthesis of n-butanol, the cell comprising:
i. a recombinant sequence encoding an acylating aldehyde dehydrogenase catalyzing the conversion of acetaldehyde to acetyl-CoA,
ii. a recombinant sequence encoding a keto-thiolase or acetyl-CoA acetyltransferase catalyzing the conversion of acetyl-CoA to acetoacetyl-CoA,
iii. a recombinant sequence encoding an acetoacetyl-CoA reductase or hydroxybutyryl-CoA dehydrogenase catalyzing the conversion of acetoacetyl-CoA to 3-hydroxybutyryl-CoA,
iv. a recombinant sequence encoding a crotonase catalyzing the conversion of 3-hydroxybutyryl-CoA to crotonyl-CoA,
v. a recombinant sequence encoding a crotonyl-CoA reductase, butyryl-CoA dehydrogenase or trans-enoyl-CoA reductase catalyzing the conversion of crotonyl-CoA to butyryl-CoA, and
vi. a recombinant sequence encoding a butyraldehyde/butanol dehydrogenase catalyzing the conversion of butyryl-CoA to n-butanol.

2. The recombinant cell of claim 1, wherein the sequences encoding the acylating aldehyde dehydrogenase, the keto-thiolase or acetyl-CoA acetyltransferase, the acetoacetyl-CoA reductase or hydroxybutyryl-CoA dehydrogenase, the crotonase, the crotonyl-CoA reductase, butyryl-CoA dehydrogenase or trans-enoyl-CoA reductase, and the butyraldehyde/butanol dehydrogenase are linked.

3. The recombinant cell of claim 1, wherein the sequences encoding the acylating aldehyde dehydrogenase, the keto-thiolase or acetyl-CoA acetyltransferase, the acetoacetyl-CoA reductase or hydroxybutyryl-CoA dehydrogenase, the crotonase, the crotonyl-CoA reductase, butyryl-CoA dehydrogenase or trans-enoyl-CoA reductase, and the butyraldehyde/butanol dehydrogenase are not linked.

4. The recombinant cell of claim 1 further comprising a recombinant sequence encoding a pyruvate decarboxylase catalyzing the conversion of pyruvate to acetaldehyde.

5. The recombinant cell of claim 4, wherein the pyruvate decarboxylase is derived from Z. mobilis or S. cerevisiae.

6. (canceled)

7. (canceled)

8. (canceled)

9. (canceled)

10. The recombinant cell of claim 1, wherein the acylating aldehyde dehydrogenase is derived from E. coli.

11. The recombinant cell of claim 10, wherein the acylating aldehyde dehydrogenase is EutE.

12. The recombinant cell of claim 1, wherein the keto-thiolase or acetyl-CoA acetyltransferase is derived from E. coli, R. eutrophus, A. caviae, T. denticola or C. acetobutylicum.

13. The recombinant cell of claim 12, wherein the keto-thiolase or acetyl-CoA acetyltransferase is PhaA, AtoB, FadA, or Th1.

14. The recombinant cell of claim 1, wherein the acetoacetyl-CoA reductase or hydroxybutyryl-CoA dehydrogenase is derived from E. coli, R. eutrophus, A. caviae, T. denticola, or C. acetobutylicum.

15. The recombinant cell of claim 14, wherein the acetoacetyl-CoA reductase or hydroxybutyryl-CoA dehydrogenase is Hbd or PhaB.

16. The recombinant cell of claim 1, wherein the crotonase is derived from E. coli, R. eutrophus, A. caviae, T. denticola, or C. acetobutylicum.

17. The recombinant cell of claim 16, wherein the crotonase is Crt or PhaJ.

18. The recombinant cell of claim 1, wherein the crotonyl-CoA reductase, butyryl-CoA dehydrogenase or trans-enoyl-CoA reductase is derived from E. coli, R. eutrophus, A. caviae, T. denticola, C. acetobutylicum or S. collinus.

19. The recombinant cell of claim 18, wherein the crotonyl-CoA reductase, butyryl-CoA dehydrogenase, or trans-enoyl-CoA reductase is Ter or Ccr.

20. The recombinant cell of claim 1, wherein the butyraldehyde/butanol dehydrogenase is derived from E. coli, R. eutrophus, A. caviae, T. denticola, or C. acetobutylicum.

21. The recombinant cell of claim 20, wherein the butyraldehyde/butanol dehydrogenase is Bcd, Aad, or AdhE2.

22. The recombinant cell of claim 4, wherein the pyruvate decarboxylase is Pdc from Z. mobilis, the acylating aldehyde dehydrogenase is EutE from E. coli, the keto-thiolase is PhaA from R. eutrophus, the hydroxybutyryl-CoA dehydrogenase is Hbd from C. acetobutylicum, the crotonase is Crt from C. acetobutylicum, the crotonyl-CoA reductase is Ter from T. denticola, and the alcohol dehydrogenase is AdhE2 from C. acetobutylicum and wherein the recombinant cell is a S. cerevisiae cell or an E. coli cell.

23. (canceled)

24. (canceled)

25. (canceled)

26. (canceled)

27. (canceled)

28. (canceled)

29. (canceled)

30. (canceled)

31. (canceled)

32. The recombinant cell of claim 1, further comprising a recombinant sequence encoding a pantothenate kinase catalyzing the conversion of pantothenate to 4′-phosphopantothenate.

33. (canceled)

34. (canceled)

35. The recombinant cell of claim 1, further comprising a recombinant sequence encoding a phosphopantothenoylcysteine synthetase catalyzing the conversion of 4′-phosphopantothenate to 4′-phosphopantothenoylcysteine.

36. (canceled)

37. (canceled)

38. The recombinant cell of claim 1, further comprising a recombinant sequence encoding phosphopantothenonylcysteine decarboxylase catalyzing the conversion of 4′-phosphopantothenoylcysteine to 4′-phosphopantetheine.

39.-70. (canceled)
